(19) United States
(12) Patent Application Publication
(10) Pub. No.: US 2018/0244777 A1
(43) Pub. Date: Aug. 30, 2018

(54) Novel Treatment Methods Based on Multifunctional Molecules

(71) Applicant: NUMAB INNOVATION AG, Wädenswil (CH)

(72) Inventor: David Urech, Jona (CH)

(73) Assignee: Numab Therapeutics AG, Pffäfikon (CH)

(21) Appl. No.: 15/575,049

(22) PCT Filed: May 18, 2016
(86) PCT No.: PCT/EP2016/000827

§ 371 Date: Nov. 17, 2017

(30) Foreign Application Priority Data
May 18, 2015 (EP) 15001493.4

(51) Int. Cl. C07K 16/28; A61P 25/00; A61K 39/00

(52) U.S. Cl. CPC C07K 16/2809; C07K 2317/34; A61P 25/00; C07K 2317/626; C07K 2317/622; C07K 2317/55; C07K 2317/35; C07K 2317/33; C07K 2317/31; C07K 2317/24; A61K 2039/572; A61K 2039/505; C07K 2317/75; C07K 2317/92; C07K 16/2866

(57) ABSTRACT

The present invention relates to novel treatment methods based on multifunctional molecules, particularly bispecific molecules, wherein the multifunctional molecules comprise an antibody, or a functional fragment thereof, with high affinity combined with high potency, particularly an antibody, or a functional fragment thereof, against a particular epitope.

### CROSS-REFERENCE TO RELATED APPLICATIONS

This application is a 371 national phase of International Patent Application No. PCT/EP2016/000827 filed May 18, 2016, which claims priority to European Patent Application No. 15001493.4 filed May 18, 2015, the content of which applications is incorporated herein by reference.

### SEQUENCE LISTING

The instant application contains a sequence listing submitted electronically via EFS-web, which serves as both the paper copy and the computer readable form (CRF) and consists of a file entitled “N-0008-USNP_seqlist.txt”, which was created on Apr. 9, 2018, which is 106,496 bytes in size, and which is herein incorporated by reference in its entirety.

### FIELD OF THE INVENTION

The present invention relates to novel treatment methods based on multifunctional molecules, particularly bispecific molecules, wherein the multifunctional molecules comprise an antibody, or a functional fragment thereof, with high affinity combined with high potency, particularly an antibody, or a functional fragment thereof, against a particular epitope.

### BACKGROUND OF THE INVENTION

Chronic inflammatory diseases, such as rheumatoid arthritis (RA), psoriasis, psoriatic arthritis (PsA), ulcerative colitis (UC), Crohn's disease (CD), ankylosing spondylitis (AS) or multiple sclerosis (MS) are held to be caused by different subsets of T-cells (mainly Th-17 and certain γδ-T cells) and certain innate lymphocytes (e.g. NK cells). Upon stimulation, these cells produce numerous effector cytokines that initiate a cascade leading to local and/or systemic inflammation, oftentimes resulting in alteration or damage of tissues.

Inhibitors of cytokines signaling cascades that are involved in the differentiation of disease-driving T cells (e.g. IL-1β, TGF-β, IL-6 and IL-23) as well as inhibitors of effector cytokines that are produced by disease-driving T cells (e.g. IFN-γ, GM-CSF, G-CSF, IL-17, TNF and IL-22) have been demonstrated to be effective in certain disease conditions. However, global elimination of individual cytokines is associated with certain disadvantages: first, it bears the risk of severe side effects as this may broadly affect immune system's ability to defend the host against pathogens. Further, redundant signaling pathways may compensate for each other. The pathogenic cell types may even become independent of certain upstream cytokines, thus leaving the patient without effective treatment.

Despite the highly variable cytokine production profile of many inflammatory cells that are critically involved in the genesis and maintenance of chronic inflammation and autoimmune diseases, it appears to be a common feature of such pathogenic cell types to express IL23R, independently of their differentiation state.

Traditional therapeutic antibody approaches are targeting selected cytokines that are believed to play a crucial role in the pathogenesis of the inflammatory process. For example, the historically most successful anti-inflammatory monoclonal antibody therapies inhibit a pro-inflammatory cytokine called tumor necrosis factor-α (TNF). Despite very good results in many patients, 20-40% of RA patients do not respond to anti-TNF therapy, which is associated with considerable adverse effects. Furthermore it has been demonstrated that Th17-like cells and Th17 cytokines increased during TNF therapy in refractory patients (Alzabin et al., Ann Rheum Dis. 2012; 71:1741-1748).

More recently, antibodies targeting either the downstream effector cytokine interleukin-17 (IL-17) or the upstream IL-23, which drives differentiation of pathogenic IL23R expressing T cells, have proven to be safe and highly efficacious in certain autoimmune diseases. For example, the anti-IL-23 antibody ustekinumab/Stelara® showed excellent safety and efficacy in psoriasis and PsA, whereas it failed in MS and CD (Longbrake et al Expert Rev Neurother. 2009; 9:319-329; Sandborn et al, Gastroenterology. 2008; 135:1130-1141). Similarly, anti-IL-17 therapies were efficacious in psoriasis and uveitis and to some extent in RA (Jones et al, Nature Immunology. 2012; 13:1022-1025); however, studies in CD with secukinumab/AIN457 were recently terminated due to lack of efficacy (Hueber et al, 2012). These results suggest that although very similar cell types are responsible for the inflammatory processes in autoimmune diseases, the cytokine pattern they produce may differ substantially between the various diseases.

By using two different IL-23-blocking antibodies in a murine experimental autoimmune encephalomyelitis (“EAE”) model for multiple sclerosis it was shown that both antibodies could completely block development of EAE symptoms in a prophylactic setting (Chen et al. J. Clin. Invest. 2009; 116:1317-1325). However, the blockade of IL-23 signaling appeared to be insufficient for the therapy of established MS, when the IL-23-blocking antibodies were administered during an exacerbation episode.

Instead of targeting a soluble cytokine, it is possible as well to target the corresponding cytokine receptor. For example, an antibody against IL23R was employed in a murine EAE model by administration 2 days after active immunization with an encephalitogenic PLP, and a prophylactic activity of the anti-IL23R could be demonstrated, since the onset of EAE could be delayed and the EAE score could be reduced (Wojkowska et al., Mediators of Inflammation 2014, Article ID 590409, 1-8).

Instead of systemically blocking selected cytokines or cytokine-receptor interactions, alternative approaches aim at specifically eliminating certain immune cells. In MS, the hypothesis that elimination of immune cells can lead to long-lasting efficacy has been supported by the recent impressive clinical results with alemtuzumab/Lemtrada®. In a follow-up study to the pivotal phase 3 trial, 80% of patients did not require any further treatment for up to 24 months after the last treatment cycle. However, Lemtrada® unspecifically eliminates the major components of the adaptive immune system (all B- and T-cells) and is therefore associated with significant side-effects—such as opportunistic infections—that would not justify its broad use in other chronic inflammatory diseases.

One of these alternative approaches aims at the elimination of activated pathogenic lymphocytes that are the producers of the various cytokines, which, for example, ultimately cause chronic inflammation (WO 2014/180577). A unifying feature of activated pathogenic T lymphocytes seems to be their expression of IL23R (Ghoreschi et al. Nature. 2010; 467:967-971; Paget et al. Immunology and Cell Biology. 2014:1-15). WO 2014/180577 employs a bi-specific antibody fragment binding on one end to IL23R on pathogenic lymphocytes and on the other end to CD3, particularly CD3ε, a component of the T-cell co-receptor complex expressed on cytotoxic T-killer cells.

The TCR is associated with other molecules like CD3, which possess three distinct chains (γ, δ, and ε) in mammals, and either a (CD247) complex or a ζ/η complex. These accessory molecules have transmembrane regions and are vital to propagating the signal from the TCR into the cell; the cytoplasmic tail of the TCR is extremely short, making it unlikely to participate in signaling. The CD3- and ζ-chains, together with the TCR, form what is known as the T cell receptor complex.

CD3E is a type I transmembrane protein expressed on the surface of certain T cells. It participates in the T cell receptor (TCR) complex and interacts with other domains of this complex. One of these interaction partners is CD3γ, which binds to CD3ε in a 1:1 stoichiometry (De la Hera et al, J. Exp. Med. 1991; 173: 7-17). FIG. 5 shows a schematic view of the TCR complex, including CD3ε/CD3γ. It is believed that binding of the TCR to the MHC-peptide complex on the surface of an antigen presenting cell (APC) and subsequent movement of the T cell along the APC leads to a certain rotation of the TCR complex resulting in a dislocation of CD3ε and CD3γ relative to each other, which is required for efficient TCR signaling and therefore activation of T-cells. Certain antibodies against CD3ε have been demonstrated to induce TCR signaling while others did not. TCR-activating antibodies typically bind to an exposed epitope on CD3ε (see FIG. 5, “agonistic epitope”), whereas some non-stimulatory antibodies have been demonstrated to bind to the interface between CD3ε and CD3γ, or to concomitantly bind to CD3ε and CD3γ (see FIG. 5, “antagonistic epitope”), thus possibly interfering with the relative displacement of CD3ε and CD3γ (Kim et al, JBC. 2009; 284: 31028-31037).

However, most of the MS patients show the relapse-remitting form of MS, which is characterized by unpredictable relapses followed by periods of varying duration without new signs of disease activity (remission). As mentioned above, many of the approaches pursued so far are of a prophylactic nature, i.e. they appear to be able to delay, and/or to alleviate the consequences of, an ensuing relapse. However, it would be highly desirable to identify a treatment option that could be administered successfully in the acute situation of a relapse.

Such acute treatment option, however, is further complicated by the fact that relapses may be accompanied by so-called “cytokine storms”, a complex scheme of cytokine-driven cascades of reactions, involving cytokines and other signalling molecules and cells of the immune system, which renders it rather unlikely that a single point of attack for a treatment option could be identified in such a situation.

Thus, there remained still a large unmet need to develop novel methods for the treatment of multiple sclerosis after the onset of an exacerbation episode, and novel compositions for use in such treatments.

The solution for this problem is provided by a molecule that eliminates the disease-driving cell types. As IL23R is specifically expressed by cells that are critically involved in the inflammatory process after the onset of and during an exacerbation episode, it qualifies as a target for cell-depleting molecules. Such depletion of could for example be achieved by a bispecific anti-IL23R/anti-CD3-binding molecules for use in a treatment comprising the administration of such bispecific molecules after the onset of, and during, an exacerbation episode experienced by an MS patient. The results achieved by such an approach, i.e. the alleviation of such exacerbation episode and its impact on the patient had not been achieved or suggested by the prior art before.

### SUMMARY OF THE INVENTION

The present invention relates to IL23R-binding molecules that lead to specific depletion of IL23R expressing cell types for use in a treatment comprising the administration of such molecules after the onset of, and during, an exacerbation episode experienced by an MS patient.

Thus, in a first aspect, the present invention relates to a multifunctional molecule for use in the treatment of multiple sclerosis after the onset of an exacerbation episode, wherein said multifunctional molecule comprises at least (i) a target-binding moiety, which is specific for IL23R; and (ii) a second functional moiety, which leads to the depletion of IL23R-expressing cells.

In a second aspect, the present invention relates to a method for the treatment of multiple sclerosis after the onset of an exacerbation episode comprising the step of administering to a patient a multifunctional molecule, wherein said multifunctional molecule comprises at least (i) a target-binding moiety, which is specific for IL23R; and (ii) a second functional moiety, which leads to the depletion of IL23R-expressing cells.

In a third aspect, the present invention relates to a multifunctional molecule for use in the prophylactic treatment of multiple sclerosis to prevent or delay an exacerbation episode, wherein said multifunctional molecule comprises at least (i) a target-binding moiety, which is specific for IL23R; and (ii) a second functional moiety, which leads to the depletion of IL23R-expressing cells.

### BRIEF DESCRIPTION OF THE DRAWINGS

FIG. 1 shows the phylogenetic clustering of joined VH and VL CDR Sequences from monoclonal rabbit antibodies.

FIG. 2 shows binding of purified monoclonal rabbit antibodies to Jurkat T cells.

FIG. 3 shows the stimulation of CD69 expression by cross-linked anti-hCD3ε mAbs (FIG. 3A: Cluster 02a; FIG. 3B: Cluster 02b). The potential of purified monoclonal rabbit anti-CD3 antibodies and comparator antibodies TR66 and OKT-3 to induce T-cell activation was assessed by measurement of CD69 expression. Three different concentrations of cross-linked antibodies were used to stimulate Jurkat cells and CD69 expression was assessed by flow-cytometry 24 h later. Antibody concentrations used were 1.25 μg/ml (a), 5.0 μg/ml (b) and 20 μg/ml (c); Number of Jurkat cells (y axis) vs. PE intensity (CD69 expression) (x axis).

FIG. 4 shows the stimulation of CD69 by cross-linked rabbit mAbs over time. The potential of purified monoclonal rabbit anti-CD3 antibodies to induce T-cell activation was assessed by measurement of CD69 expression. Cross-linked antibodies were used at a concentration of 5.0 μg/ml to stimulate Jurkat cells and CD69 expression was assessed by flow-cytometry 0, 4, 15, 24, 48 and 72 h later. For the qualitative detection of CD69 expression the mean fluorescence intensity (MFI), reflecting the signal intensity at the geometric mean, was measured for both, the negative control as well as for the test antibodies. The difference of the MFI between test antibody and negative control (ΔMFI) was calculated as a measure for CD69 expression.

FIG. 5 shows a simplified schematic view of the TCR complex, including CD3ε/CD3γ.

FIG. 6 shows the results of epitope mapping experiments for prior art antibodies: (a) epitope mapping of antibody SP34 (see file history of EP 2 155 788); (b) epitope mapping of Micromet antibody (see EP 2 155 788/WO 2008/119567; FIG. 6 shows the results of binding experiments of single alanine mutants, where a decrease of binding for a given mutant indicates the relevance of the corresponding wild-type amino acid residue for antibody binding (i.e. low bar=highly relevant for binding).

FIG. 7 shows the results of epitope mapping experiments by ELISA for antibodies of the present invention (clone-02, clone-03, clone-06); FIG. 7 shows the results of binding experiments in a peptide scan analysis. 15mer linear arrays derived from human CD3ε, residues 1-15 in which each position is substituted by 18 amino acids (all natural amino acids except cysteine) were probed with 0.1 μg/ml of each antibody to study amino acid specificities affecting binding to the epitope. Decrease in binding signals in ELISA is given, (a) for each substitution individually, and (b) averaged over the 18 different substitutions for each position. The height of a bar in FIG. 7b indicates the relevance of the corresponding wild-type amino acid residue for antibody binding (i.e. large bar=highly relevant for binding).

FIG. 8 shows binding of anti-CD3×anti-IL5R scDbs to Jurkat T-cells and CHO-IL5R cells. Binding of A) Construct 1, B) Construct 2 and C) Construct 3 to Jurkat T-cells and CD3-negative Jurkat cells and binding of D) Construct 1, E) Construct 2 and F) Construct 3 to IL5R-CHO cells as well as wild-type CHO cells was assessed by flow cytometry. Construct 1, Construct 2 and Construct 3 have the same anti-IL5R moiety but 3 different anti-CD3 moieties that bind to CD3 with diverse affinities (1.15×10⁻⁸ M for Construct 1, 2.96×10⁻⁸ M for Construct 2, and 1.23×10⁻⁷ M for Construct 3); Construct 1=comprises the humanized variable domain of clone-06; Construct 2=comprises the humanized variable domain of clone-02; Construct 3=comprises the humanized variable domain of clone-03.

FIG. 9 shows the specific stimulation of interleukin-2 secretion by cross-linking of cytotoxic T-cells with target cells by scDbs. CD8+ T-cells were incubated with increasing concentrations of scDbs in presence of CHO-IL5R or CHO cells. Interleukin-2 concentrations in culture supernatants were measured by ELISA after 16 hours of incubation; Construct 1=comprises the humanized variable domain of clone-06; Construct 2=comprises the humanized variable domain of clone-02; Construct 3=comprises the humanized variable domain of clone-03.

FIG. 10 shows the specific lysis of human IL5R-expressing CHO cells by anti-CD3×anti-IL5R scDbs. CD8+ T-cells were incubated with increasing concentrations of scDbs in presence of CHO-IL5R or CHO cells. Target cells (CHO-IL5R and CHO) were labeled with cell tox green dye and cell lysis was determined by measurement of fluorescence intensity after 88 hours of incubation; Construct 1=comprises the humanized variable domain of clone-06; Construct 2=comprises the humanized variable domain of clone-02; Construct 3=comprises the humanized variable domain of clone-03.

FIG. 11 shows the dose-dependence of target cell lysis by CD8+ T cells redirected by bispecific anti-IL5R×CD3 scDbs. Human IL5R expressing CHO cells were incubated with naïve human CD8+ cells (E:T=10:1) and increasing concentrations of scDbs. Two independent experiments (A and B) with CD8+ T cells from different donors are shown. Both scDbs contain identical anti-IL5R domains but different anti-CD3 domains. The scDb containing the Numab anti-CD3 variable domain (humanized clone 6) shows higher maximal lysis at every time point and concentration tested. Maximal lysis with this scDb is reached at lower concentrations as compared to the scDb containing the TR66 anti-CD3 domain. Further, in contrast to the TR66-containing scDb, which at high concentrations shows almost complete inhibition of target cell lysis (particularly at early time points), the scDb with the anti-CD3 domain derived from clone 6 shows maintained lysis activity at high scDb concentrations.

FIG. 12 shows the correlation of dose-dependence of target cell lysis and cytokine production by redirected CD8+ T cells. Human IL5R expressing CHO cells were incubated with naïve human CD8+ cells (E:T=10:1) and increasing concentrations of scDbs for 64 hours. While the potency of both anti-IL5R×CD3 scDbs either containing the Numab anti-CD3 variable domain (humanized clone 6) or the variable domain of TR66 is very similar (A), there is a profound difference in the dose-dependence of cytokine production, exemplified by TNFα (B) and IFNγ (C). For example at 0.8 nM the scDb containing the Numab anti-CD3 domain shows greater lysis than the scDb containing TR66, while both cytokines, TNFα and IFNγ reach only about 50% of the concentrations measured with the TR66 containing scDb. In line with the observed drop in target cell lysis at high concentrations of the TR66 containing scDb, also the production of IFNγ and TNFα decreased at high concentrations.

FIG. 13 shows the potency of anti-IL5R×CD3 scDbs to induce expression of the early T cell activation marker CD69 on CD8+ T cells in presence or absence of IL5R expressing CHO cells. Human IL5R+ and hIL5R-CHO cells were incubated with naïve human CD8+ cells (E:T=10:1) and increasing concentrations of scDbs for 18 hours. CD69 expression was assessed by flow-cytometry after 18 hours of incubation. Both scDbs, either containing the Numab anti-CD3 variable domain (humanized clone 6) or the variable domain of TR66 showed very similar potency to activate CD8+ T cells, with the Numab anti-CD3 showing slightly weaker induction of CD69 expression at low concentration. In contrast to TR66, for which CD69 expression dropped after a peak at 4 nM, CD69 expression steadily increased with increasing concentration for the Numab anti-CD3.

FIG. 14 shows clinical scores in the active induced mouse EAE model. SJL mice were treated with PRO386 and PRO387 in a prophylactic setting, before the onset of the disease.

FIG. 15 shows clinical scores in the active induced mouse EAE model. SJL mice were treated with PRO386, PRO387, anti-CD3 Fab and PBS in a therapeutic setting after the onset of the disease.

FIG. 16 shows that an IL-12/23 inhibitory anti-p40 mAb (Stelara-like) lacks therapeutic efficacy even at a much higher dose in contrast to PRO386. Timeline of clinical symptom progression: Mice treated with 50 μg/d CD3FAB or 200 μg/d anti-p40 antibody starting after disease onset (clinical score of one) on day 17 displayed progressing paralysis. However, injection of 50 μg/d PRO386 after the disease onset (clinical score of one) prevented disease progression and even ameliorated disease symptoms. Mice treated with 200 μg/d anti-p40 antibody just after the cell transfer showed no disease symptoms. This suggests that disease progression becomes independent of IL23 signaling after manifestation of clinical symptoms.

FIGS. 17 (A) and (B) show the cytokine production by CNS infiltrating CD4 T cells from C57B/6 mice which were transferred with 10¹⁰ lymphocytes from previously MOG/CFA immunized C57B/6 animals. Quantification of frequencies from CNS infiltrating cytokine producing CD4 T cells (A), which were pre-gated on CD45+, singlet, live and CD4+ cells analysed by flow cytometry. Quantification of absolute numbers of CNS infiltrating CD4 T cells (B) by flow cytometry. Cells were pre-gated as in (A). Each dot represents one mouse. Results represent mean+/−SEM. For analysis of EAE score plots comparing PBS-treated group against the PRO386-treated group, 2 way ANOVA with Bonferroni's post-test was used.

FIG. 18 shows the results from treating mice which were injected 7.5×10⁶ lymphocytes from previously immunized animals. The time course of clinical manifestation of the disease is depicted. Results represent mean+/−SEM. N per group 4-5 animals. For analysis of EAE score plots comparing PBS treated group against the CD3FAB, PRO386 (prevention) and PRO386 (therapy) treated group, 2 way ANOVA with Bonferroni's post-test was used.

### DETAILED DESCRIPTION OF THE INVENTION

The present invention relates to multifunctional molecules comprising IL23R-binding molecules that lead to specific depletion of IL23R-expressing cell types for use in a treatment comprising the administration of such multifunctional molecules after the onset of, and during, an exacerbation episode experienced by an MS patient.

Thus, in a first aspect, the present invention relates to a multifunctional molecule for use in the treatment of multiple sclerosis after the onset of an exacerbation episode, wherein said multifunctional molecule comprises at least (i) a target-binding moiety, which is specific for IL23R; and (ii) a second functional moiety, which leads to the depletion of IL23R-expressing cell.

In a second aspect, the present invention relates to a method for the treatment of multiple sclerosis after the onset of an exacerbation episode comprising the step of administering to a patient a multifunctional molecule, wherein said multifunctional molecule comprises at least (i) a target-binding moiety, which is specific for IL23R; and (ii) a second functional moiety, which leads to the depletion of IL23R-expressing cells.

In another aspect, the present invention relates to a multifunctional molecule, wherein said multifunctional molecule comprises at least (i) a target-binding moiety, which is specific for IL23R; and (ii) a second functional moiety, which leads to the depletion of IL23R-expressing cell.

Binding of the molecule to IL23R-expressing cells results in the depletion of such cells, for example via engagement of T-killer cells to lyse the IL23R-expressing cells, thus eliminating pathogenic disease-causing lymphocytes. This approach results in better and broader efficacy as compared to blockade of individual cytokines, and in a reset of the misguided immune system, consequently leading to a long lasting improvement of the disease state. Such a multifunctional molecule, such as an anti-IL23R×CD3 bi-specific antibody fragment, very selectively targets a pathogenic subpopulation of activated leukocytes, the elimination of which is not expected to limit the protection afforded by the immune system as such, as a broad repertoire of naive and responsive B- and T-cells will be maintained. For example in the gamma-delta T cell compartment only a small subpopulation of CD27 negative/CD3 bright cells is expressing the typical Th17 cytokines and it is only this population that is positive for IL23R (Paget et al. Immunology and Cell Biology. 2014:1-15; Chognard et al. PLOS ONE. 2014; 91-15). Besides the gamma-delta T cells it has been shown in IL23R-GFP reporter mice that IL23R expression is specific to IL-17 producing CD4+ alpha-beta T cells and certain IL-17 producing myeloid cells (Awasthi et al. J Immunol. 2009; 182:1-11). Due to the restricted expression of IL23R on pathogenic inflammatory cells the specific elimination of IL23R expressing pathogenic cells will be even safer than the more traditional blockade of individual cytokines (e.g. TNF) that are globally involved in immune processes, particularly because only a very small fraction of T cells is indeed expressing IL23R.

Specific elimination of the disease driving Th17 cells in MS holds the potential to show similar efficacy as alemtuzumab/Lemtrada® with better safety though. It has been demonstrated that expression of IL-23 and IL-17 is upregulated in lesions and the percentage of IL-17 expressing cells correlates with disease activity (Tzartos et al, Neurbiology. 2008:172:146-155; Kebir et al, Nature medicine. 2007; 13:1173-1175) and further, that Th17 but not Th1 cells are increased in the cerebrospinal fluid (CSF) of MS patients (Brucklacher-Waldert et al. Brain. 2009:132; 3329-2241). Further, it has been demonstrated that the Th17 cytokines IL-17 and IL-22 disrupt the blood brain barrier, which is a prerequisite for the invasion of inflammatory cells into the CNS (Kebir et al. Nature Medicine. 2007; 10:1173-1175). In a monkey EAE model for multiple sclerosis, the IL-23 inhibitory antibody Stelara, which interferes with differentiation and activation of Th17 cells, showed good efficacy (Brok H P M et al. J Immunol 2002; 169:6554-6563). Further, preclinical animal studies suggest that the memory of the disease (EAE) lies in the Th17 compartment, indicating that specific elimination of Th17 cells could result in a long-lasting amelioration of the disease phenotype (McGeachy et al, Nat Immunol. 2009 March; 10(3): 314-324. doi:10.1038/ni.1698. and Haines et al, Cell Rep. 2013 Apr. 24. pii: S2211-1247(13)00159-9. doi: 10.1016/j.celrep.2013.03.035.). Despite the very strong scientific support for the role of Th17 cells in MS, the interleukin-12/23 inhibiting antibody Stelara did not show significant responses in a phase II clinical study in MS. A probable explanation for this is the unfortunate patient inclusion criteria in this phase II study (Longbrake E and Racke M K Expert Rev Neurother. 9(3), 319-321 (2009)). The enrolled patients had advanced disease, in which Th17 cells have already terminally differentiated, and terminally differentiated Th17 cells are no longer dependent on IL-23 signaling, do however continue to express the IL-23 receptor (McGeachy M J Nat Immuno. 2009; 10:314-324). In contrast to Stelara, the present approach targets such terminally differentiated Th17 cells. The experiments presented herein with a mouse surrogate for the bispecific constructs in accordance with the present invention have indeed demonstrated efficacy not only in a prevention setting but also by a therapeutic intervention starting after the onset of the acute phase of the disease when Th17 cells have already terminally differentiated. This is in sharp contrast to the lack of efficacy of two different IL-23-blocking antibodies in the same therapeutic setting (Chen et al. J. Clin. Invest. 2009; 116:1317-1325). Hence, the blockade of IL-23 signaling appears to be insufficient for the therapy of established MS, while depletion of IL23R expressing cells is showing strong effects. The T cell receptor or TCR is a molecule found on the surface of T lymphocytes (or T cells) that is responsible for recognizing antigens bound to major histocompatibility complex (MHC) molecules on the surface of antigen presenting cells (APC). The binding between TCR and antigen is of relatively low affinity. When the TCR engages with antigen and MHC, the T lymphocyte is activated through a series of biochemical events mediated by associated enzymes, co-receptors, specialized accessory molecules, and activated or released transcription factors.

In the context of the present invention, the term “multifunctional molecule” refers to a molecule comprising at least two functional moieties, e.g. at least two binding sites, which are specific for a cognate target. The definition of this term thus includes, but is not limited to, bispecific molecules consisting of two binding sites, such as a single-chain diabody (scDb), bispecific molecules comprising two or more copies of at least one binding site, such as a tandem scDb (Tandab); or trispecific molecules consisting of three binding sites, such as a tribody or triabody. In particular embodiments, the multifunctional molecule is a bispecific molecule. In other particular embodiments, the multifunctional molecule is a trispecific molecule.

In particular embodiments of the aspects of the present invention, said binding molecule is an antibody or a functional fragment thereof.

In particular embodiments of the aspects of the present invention, the cell presenting the target for said target-binding-moiety is a pathogenic cell, particularly a cell selected from the group consisting of (i) a T cell expressing the transcription factor RORγ(t), (ii) a T cell producing GM-CSF and/or IFN gamma, and/or IL-17, particularly an IL-17 producing T cell (Th17 cell), (iii) a γδ T cell, (iv) a natural killer T (NKT) cell, and (v) an invariant natural killer (iNK) cell; particularly a Th17 cell or a γδ T cell.

The transcription factor RORγ(t) promotes thymocyte differentiation into pro-inflammatory Th17 cells and also plays a role in inhibiting apoptosis of undifferentiated T cells and promoting their differentiation into Th17 cells, possibly by down-regulating the expression of the Fas ligand and IL-2, respectively. In particular embodiments, the cells are selected from the group consisting of IL-17 producing T cells (Th17 cells), γδ T cells, natural killer T (NKT) cells and invariant natural killer (iNK) cells.

In particular embodiments of the aspects of the present invention, said second functional moiety specifically binds to a first antigen present on a cytotoxic effector T (Tc) cell, particularly wherein said Tc cell is a stimulated or an unstimulated Tc cell.

In particular embodiments of the aspects of the present invention, said second functional moiety specifically binds to an antigen selected from CD3 and CD28.

In particular embodiments of the aspects of the present invention, said second functional moiety is a binding molecule comprising a binding region that is specific for an epitope of human CD3, particularly for an epitope of the epsilon chain of human CD3 (CD3ε), more particularly to an agonistic epitope of CD3ε.

In particular embodiments, said binding molecule, particularly said antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, said binding molecule, in particular said antibody or functional fragment thereof, is binding to human CD3 with an equilibrium dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M.

In particular embodiments, said binding molecule is an antibody or a functional fragment thereof, which, when tested in an IgG format, upon cross-linking, is inducing T-cell activation at least 1.5-fold stronger than antibodies OKT-3 or TR66 after 24 h of stimulation at an IgG concentration of 1.25 μg/ml.

In particular embodiments, said binding molecule is an antibody or a functional fragment thereof, which, when tested in an IgG format upon cross-linking, is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml.

In particular embodiments, said binding molecule is an antibody or a functional fragment thereof, which, when tested in an IgG format, upon cross-linking, is resulting in a dose-dependent activation state of T-cells that is less heterogeneous when compared to activation by OKT-3 or TR66.

In particular embodiments, the binding molecule is a CD3-binding molecule that is specific for an epitope of human CD3, wherein said CD3-binding molecule is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M, in particular to an antibody or a functional fragment thereof comprising an antigen-binding region that is specific for an epitope of human CD3, wherein said antibody or functional fragment thereof, is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M.

In particular embodiments, said binding molecule, particularly said antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, the binding molecule is an antibody or a functional fragment thereof comprising an antigen-binding region that is specific for an epitope of human CD3, wherein said antibody or functional fragment thereof, when tested in an IgG format, upon cross-linking, is inducing T-cell activation at least 1.5-fold stronger than antibodies OKT-3 or TR66 after 24 h of stimulation at an IgG concentration of 1.25 μg/ml.

In particular embodiments, said binding molecule, particularly said antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, the binding molecule is an antibody or a functional fragment thereof comprising an antigen-binding region that is specific for an epitope of human CD3, wherein said antibody or functional fragment thereof, when tested in an IgG format upon cross-linking, is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml.

In particular embodiments, said binding molecule, particularly said antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, the binding molecule is an antibody or a functional fragment thereof comprising an antigen-binding region that is specific for an epitope of human CD3, wherein said antibody or functional fragment thereof, when tested in an IgG format, upon cross-linking, is resulting in a dose-dependent homogeneous activation state of T-cells.

In particular embodiments, said binding molecule, particularly said antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, the binding molecule is an antibody or a functional fragment thereof comprising an antigen-binding region that is specific for an epitope of human CD3, wherein said antibody or functional fragment thereof, when tested in an IgG format, (i) is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M; and (iia), upon cross-linking, is inducing T-cell activation at least 1.5-fold stronger than antibodies OKT-3 or TR66 after 24 h of stimulation at an IgG concentration of 1.25 μg/ml; (iib) is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml; (iic) is resulting in a dose-dependent homogeneous activation state of T-cells; and/or (iid) is specific for an epitope of human CD3ε, wherein said epitope comprises amino acid residue N4 as residue that is critical for binding.

In particular embodiments, the binding molecule is an antibody or a functional fragment thereof comprising an antigen-binding region that is specific for an epitope of human CD3, wherein said multifunctional molecule exhibits a potency resulting in similar or even more efficient lysis of target cells when compared to a multifunctional construct comprising TR66 as CD3-binding moiety in the same format as said multifunctional molecule, while simultaneously resulting in lower production of cytokines.

In the context of the present invention, the term “potency” refers to a combination of the ED₅₀ concentration and the degree of cell lysis. Furthermore, in the context of the present invention the term “lower production of cytokines” refers to the fact that the level of cytokines in the medium, measured at the lowest concentration of the multifunctional molecule of this invention that results in maximal lysis of target cells, using a method well known to the expert (e.g. ELISA), is 10%, preferably 20%, more preferably 35% and most preferably 50% lower as compared to the same multifunctional molecule containing TR66 as CD3-binding domain.

In particular such embodiments, said antibody or functional fragment thereof, is additionally cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In the context of the present invention, the term “antibody” is used as a synonym for “immunoglobulin” (Ig), which is defined as a protein belonging to the class IgG, IgM, IgB, IgA, or IgD (or any subclass thereof), and includes all conventionally known antibodies and functional fragments thereof. A “functional fragment” of an antibody/immunoglobulin is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and Ito 113 of VH; numbering according to WO 97/08320). In the case of rabbit antibodies, the CDR regions are indicated in Table 5 (see below). A preferred class of immunoglobulins for use in the present invention is IgG. “Functional fragments” of the invention include the domain of a F(ab′)2 fragment, a Fab fragment and scFv. The F(ab′)2 or Fab may be engineered to minimize or completely remove the intermolecular disulphide interactions that occur between the CH1 and CL domains.

As used herein, a binding molecule is “specific to/for”, “specifically recognizes”, or “specifically binds to” a target, such as human CD3 (or an epitope of human CD3), when such binding molecule is able to discriminate between such target biomolecule and one or more reference molecule(s), since binding specificity is not an absolute, but a relative property. In its most general form (and when no defined reference is mentioned), “specific binding” is referring to the ability of the binding molecule to discriminate between the target biomolecule of interest and an unrelated biomolecule, as determined, for example, in accordance with a specificity assay methods known in the art. Such methods comprise, but are not limited to Western blots, ELISA, RIA, ECL, IRMA tests and peptide scans. For example, a standard ELISA assay can be carried out. The scoring may be carried out by standard colour development (e.g. secondary antibody with horseradish peroxide and tetramethyl benzidine with hydrogen peroxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. Typically, determination of binding specificity is performed by using not a single reference biomolecule, but a set of about three to five unrelated biomolecules, such as milk powder, BSA, transferrin or the like. In particular embodiments, determination of binding specificity is performed by using the set of milk powder, BSA, and transferrin as reference.

In the context of the present invention, the term “about” or “approximately” means between 90% and 110% of a given value or range.

However, “specific binding” also may refer to the ability of a binding molecule to discriminate between the target biomolecule and one or more closely related biomolecule(s), which are used as reference points. Additionally, “specific binding” may relate to the ability of a binding molecule to discriminate between different parts of its target antigen, e.g. different domains, regions or epitopes of the target biomolecule, or between one or more key amino acid residues or stretches of amino acid residues of the target biomolecule. Thus, in particular embodiments, specific binding to a particular epitope on a human target does not exclude, or even mandates, binding to non-human targets in a situation, where the non-human target comprises the identical, or at least very similar, epitope.

In the context of the present invention, the term “epitope” refers to that part of a given target biomolecule that is required for specific binding between the target biomolecule and a binding molecule. An epitope may be continuous, i.e. formed by adjacent structural elements present in the target biomolecule, or discontinuous, i.e. formed by structural elements that are at different positions in the primary sequence of the target biomolecule, such as in the amino acid sequence of a protein as target, but in close proximity in the three-dimensional structure, which the target biomolecule adopts, such as in the bodily fluid.

In one embodiment, the epitope is located on the epsilon chain of human CD3.

In certain embodiments, said binding to human CD3ε is determined by determining the affinity of said antibody or functional fragment thereof in an IgG format to the purified extracellular domain of heterodimeric CD3εγ of human origin using a surface plasmon resonance experiment.

In a particular embodiment, the following conditions are used, as shown in Example 1: MASS-1 SPR instrument (Sierra Sensors); capture antibody: antibody specific for the Fc region of said IgG immobilized on an SPR-2 Affinity Sensor chip, Amine, Sierra Sensors, using a standard amine-coupling procedure; two-fold serial dilutions of human heterodimeric single-chain CD3εγ extracellular domain ranging from 90 to 2.81 nM, injection into the flow cells for 3 min and dissociation of the protein from the IgG captured on the sensor chip for 5 min, surface regeneration after each injection cycle with two injections of 10 mM glycine-HCl, calculation of the apparent dissociation (kd) and association (ka) rate constants and the apparent dissociation equilibrium constant (K_{D}) with the MASS-1 analysis software (Analyzer, Sierra Sensors) using one-to-one Langmuir binding model.

In particular embodiments, said inducing of T-cell activation according to (iia) and/or (iic) is determined by determining the stimulation of CD69 expression by said antibody or functional fragment thereof in an IgG format.

In a particular embodiment, the following conditions are used, as shown in Example 3: stimulation of Jurkat cells (100,000 cells/well) for 24 h with 20 μg/ml, 5 μg/ml and 1.25 μg/ml of said antibody or functional fragment thereof in an IgG format after prior cross-linking by addition of 3-fold excess of an anti-IgG antibody (control: OKT3 (BioLegend, Cat. No. 317302) or TR66 (Novus Biologicals, Cat. No. NBP1-97446), cross-linking with rabbit anti-mouse IgG antibody (Jacksonlmmuno Research, Cat. No. 315-005-008)); cell staining for CD69 expression after stimulation using a Phycoerithrin (PE)-labeled antibody specific for human CD69 (BioLegend, Cat. No. 310906), analysis with a flow cytometer (FACS aria III, Becton Dickinson); negative control: unstimulated Jurkat cells incubated with the cross-linking antibody stained with said anti-CD69 antibody.

In particular embodiments, said longer lasting T-cell activation according to (iib) is determined by determining the time course of stimulation of CD69 expression by said antibody or functional fragment thereof in an IgG format.

In a particular embodiment, the following conditions are used, as shown in Example 3: stimulation of 100,000 Jurkat cells/well for 0 h, 4 h, 15 h, 24 h, 48 h and 72 h with 5 μg/ml of said antibody or functional fragment thereof in an IgG format anti-CD3 antibodies that have been cross-linked as in [0200] and analysis of CD69 expression by flow cytometry as in [0200].

In particular embodiments, said inducing of T-cell activation according to (iia) and/or (iic) is determined by determining the stimulation of IL-2 secretion by said antibody or functional fragment thereof in an IgG format.

In a particular embodiment, the following conditions are used, as shown in Example 4: stimulation of Jurkat cells (200,000 cells/well) with said antibody or functional fragment thereof in an IgG format at a concentration of 5 μg/ml using 4 different assay setups: (a) stimulation of Jurkat cells with said antibody or functional fragment thereof in an IgG format cross-linked by addition of 3-fold higher concentrations of an anti IgG antibody (control: OKT3 (BioLegend, Cat. No. 317302) or TR66 (Novus Biologicals, Cat. No. NBP1-97446), cross-linking with rabbit anti-mouse IgG antibody (Jacksonlmmuno Research, Cat. No. 315-005-008)); (b) T-cell activation in absence of cross-linking antibody; (c) immobilization of said cross-linking antibodies on the tissue culture plates by over-night incubation; (d) immobilization of said antibody or functional fragment thereof in an IgG format (or of control antibodies) on the tissue culture plate by over-night incubation in absence of cross-linking antibodies; in each setup, one hour after addition, stimulation of cells with 10 ng/ml PMA and collection of supernatant after 24, 48 and 72 h to measure IL-2 release, quantified using a commercially available ELISA (BioLegend, Cat. No. 431801).

In particular embodiments, the antibody or functional fragment thereof is (i) a rabbit antibody or a functional fragment thereof, or (ii) an antibody or a functional fragment thereof obtained by humanizing the rabbit antibody or functional fragment thereof of (i).

Methods for the humanization of rabbit antibodies are well known to anyone of ordinary skill in the art (see, for example, Borras et al., J Biol Chem. 2010 Mar. 19; 285(12):9054-66; Rader et al, The FASEB Journal, express article 10.1096/fj.02-0281fje, published online Oct. 18, 2002; Yu et al (2010) A Humanized Anti-VEGF Rabbit Monoclonal Antibody Inhibits Angiogenesis and Blocks Tumor Growth in Xenograft Models. PLoS ONE 5(2): e9072. doi:10.1371/journal.pone.0009072).

In particular embodiments, said antibody or functional fragment thereof comprises an antigen-binding region comprising a VH domain comprising a combination of one CDR1, one CDR2 and one CDR3 region present in SEQ ID NOs: 2, 4, 6, 8, 10, 12, 14, 16, 18, and 20, particularly SEQ ID NOs: 4, 6, and 10, more particularly SEQ ID NO: 10, particularly wherein said VH domain comprises framework domains selected from the framework domains present in SEQ ID NOs: 2, 4, 6, 8, 10, 12, 14, 16, 18, and 20, particularly SEQ ID NOs: 4, 6, and 10, more particularly SEQ ID NO: 10, and a VL domain comprising a combination of one CDR1, one CDR2 and one CDR3 region present in SEQ ID NOs: 1, 3, 5, 7, 9, 11, 13, 15, 17, and 19, particularly SEQ ID NOs: 3, 5, and 9, more particularly SEQ ID NO: 9, particularly wherein said VL domain comprises framework domains selected from the framework domains present in SEQ ID NOs: 1, 3, 5, 7, 9, 11, 13, 15, 17, and 19, particularly SEQ ID NOs: 3, 5, and 9, more particularly SEQ ID NO: 9. In particular embodiments, the VL domain comprises framework domains selected from the framework domains present in SEQ ID NOs: 21, 23; and 24; and the VH domain comprises framework domains selected from the framework domains present in SEQ ID NO: 22. In other particular embodiments, the VL domain comprises framework domains that are variants of the framework domains present in SEQ ID NOs: 21, 23; and 24; and/or the VH domain comprises framework domains that are variants of the framework domains present in SEQ ID NO: 22, particularly variants comprising one or more non-human donor amino acid residues, particularly donor amino acid residues present in one of the sequences selected from SEQ ID NOs: 1 to 20, instead of the corresponding human acceptor amino residues present in SEQ ID NOs: 21, 23, 24, and/or 22.

In particular embodiments, said antibody or functional fragment thereof comprises an antigen-binding region comprising a VH domain comprising the combination of CDR1, CDR2 and CDR3 present in one of SEQ ID NOs: 2, 4, 6, 8, 10, 12, 14, 16, 18, and 20, particularly SEQ ID NOs: 4, 6, and 10, more particularly SEQ ID NO: 10, particularly wherein said VH domain comprises the combination of framework domains present in one of SEQ ID NOs: 2, 4, 6, 8, 10, 12, 14, 16, 18, and 20, particularly SEQ ID NOs: 4, 6, and 10, more particularly SEQ ID NO: 10, and a VL domain comprising the combination of CDR1, CDR2 and CDR3 present in one of SEQ ID NOs: 1, 3, 5, 7, 9, 11, 13, 15, 17, and 19, particularly SEQ ID NOs: 3, 5, and 9, more particularly SEQ ID NO: 9, particularly wherein said VL domain comprises the combination of framework domains present in one of SEQ ID NOs: 1, 3, 5, 7, 9, 11, 13, 15, 17, and 19, particularly SEQ ID NOs: 3, 5, and 9, more particularly SEQ ID NO: 9. In particular embodiments, the VL domain comprises framework domains selected from the framework domains present in SEQ ID NOs: 21, 23; and 24; and the VH domain comprises framework domains selected from the framework domains present in SEQ ID NO: 22. In other particular embodiments, the VL domain comprises framework domains that are variants of the framework domains present in SEQ ID NOs: 21, 23; and 24; and/or the VH domain comprises framework domains that are variants of the framework domains present in SEQ ID NO: 22, particularly variants comprising one or more non-human donor amino acid residues, particularly donor amino acid residues present in one of the sequences selected from SEQ ID NOs: 1 to 20, instead of the corresponding human acceptor amino residues present in SEQ ID NOs: 21, 23, 24, and/or 22.

In particular embodiments, said antibody or functional fragment thereof comprises an antigen-binding region comprising a VH domain selected from SEQ ID NOs: 2, 4, 6, 8, 10, 12, 14, 16, 18, and 20, particularly SEQ ID NOs: 4, 6, and 10, more particularly SEQ ID NO: 10, and a VL domain selected from SEQ ID NOs: 1, 3, 5, 7, 9, 11, 13, 15, 17, and 19, particularly SEQ ID NOs: 3, 5, and 9, more particularly SEQ ID NO: 9. In other particular embodiments, the VH domain is a variant of a VH domain selected from SEQ ID NOs: 2, 4, 6, 8, 10, 12, 14, 16, 18, and 20, particularly SEQ ID NOs: 4, 6, and 10, more particularly SEQ ID NO: 10, and/or the VL domain is a variant of a VL domain selected from SEQ ID NOs: 1, 3, 5, 7, 9, 11, 13, 15, 17, and 19, particularly SEQ ID NOs: 3, 5, and 9, more particularly SEQ ID NO: 9, particularly a variant comprising one or more amino acid residue exchanges in the framework domains and/or in CDR residues not involved in antigen binding.

Methods for the identification of amino acid residues in framework regions suitable for exchange, e.g. by homologous amino acid residues, are well known to one of ordinary skill in the art, including, for example, analysis of groups of homologous sequences for the presence of highly conserved residues (which are particularly kept constant) and variegated sequence positions (which may be modified, particularly by one of the residues naturally found at that position).

Methods for the identification of an amino acid residues in the CDR regions suitable for exchange, e.g. by homologous amino acid residues, are well known to one of ordinary skill in the art, including, for example, analysis of structures of antibody binding domains, particularly of structures of antibody binding domains in a complex with antigens for the presence of antigen-interacting residues (which are particularly kept constant) and sequence positions not in contact with the antigen (which may be modified).

In particular other embodiments, said antibody or functional fragment thereof comprises an antigen-binding region comprising a VH domain selected from SEQ ID NOs: 2, 4, 6, 8, 10, 12, 14, 16, 18, 20, and 22, particularly SEQ ID NOs: 4, 6, 10, and 22, more particularly SEQ ID NO: 10, and 22, and a VL domain selected from SEQ ID NOs: 1, 3, 5, 7, 9, 11, 13, 15, 17, 19, 21, 23, and 24, particularly SEQ ID NOs: 3, 5, 9, 21, 23, and 24, more particularly SEQ ID NOs: 9, 21, 23, and 24. In other particular embodiments, the VH domain is a variant of a VH domain selected from SEQ ID NOs: 2, 4, 6, 8, 10, 12, 14, 16, 18, 20, and 22, particularly SEQ ID NOs: 4, 6, 10, and 22, more particularly SEQ ID NO: 10 and 22, and/or the VL domain is a variant of a VL domain selected from SEQ ID NOs: 1, 3, 5, 7, 9, 11, 13, 15, 17, 19, 21, 23, and 24, particularly SEQ ID NOs: 3, 5, 9, 21, 23, and 24, more particularly SEQ ID NOs: 9, 21, 23, and 24, particularly a variant comprising one or more amino acid residue exchanges in the framework domains and/or in CDR residues not involved in antigen binding.

In particular embodiments, said antibody or functional fragment thereof comprises an antigen-binding region comprising a VH/VL domain combination selected from SEQ ID NO: 1/SEQ ID NO: 2; SEQ ID NO: 3/SEQ ID NO: 4; SEQ ID NO: 5/SEQ ID NO: 6; SEQ ID NO: 7/SEQ ID NO: 8, SEQ ID NO: 9/SEQ ID NO: 10, SEQ ID NO: 11/SEQ ID NO: 12, SEQ ID NO: 13/SEQ ID NO: 14, SEQ ID NO: 15/SEQ ID NO: 16, SEQ ID NO: 17/SEQ ID NO: 18, and SEQ ID NO: 19/SEQ ID NO: 20, particularly SEQ ID NO: 3/SEQ ID NO: 4; SEQ ID NO: 5/SEQ ID NO: 6; and SEQ ID NO: 9/SEQ ID NO: 10, more particularly SEQ ID NO: 9/SEQ ID NO: 10. In particular other embodiments, said antibody or functional fragment thereof comprises an antigen-binding region comprising a variant of a VH/VL domain combination selected from SEQ ID NO: 1/SEQ ID NO: 2; SEQ ID NO: 3/SEQ ID NO: 4; SEQ ID NO: 5/SEQ ID NO: 6; SEQ ID NO: 7/SEQ ID NO: 8, SEQ ID NO: 9/SEQ ID NO: 10, SEQ ID NO: 11/SEQ ID NO: 12, SEQ ID NO: 13/SEQ ID NO: 14, SEQ ID NO: 15/SEQ ID NO: 16, SEQ ID NO: 17/SEQ ID NO: 18, and SEQ ID NO: 19/SEQ ID NO: 20, particularly SEQ ID NO: 3/SEQ ID NO: 4; SEQ ID NO: 5/SEQ ID NO: 6; and SEQ ID NO: 9/SEQ ID NO: 10, more particularly SEQ ID NO: 9/SEQ ID NO: 10, wherein in such variant at least the VL or the VH domain is a variant of one of the VL/VH domains listed.

In a particular embodiment, said antibody or functional fragment thereof comprises an antigen-binding region comprising a VH/VL domain combination selected from SEQ ID NO: 21/SEQ ID NO: 22, SEQ ID NO: 23/SEQ ID NO: 22, SEQ ID NO: 24/SEQ ID NO: 22; and SEQ ID NO: 35/SEQ ID NO: 36. In another embodiment, said antibody or functional fragment thereof comprises a variant of the antigen-binding region comprising a VH/VL domain combination selected from SEQ ID NO: 21/SEQ ID NO: 22, SEQ ID NO: 23/SEQ ID NO: 22, SEQ ID NO: 24/SEQ ID NO: 22, and SEQ ID NO: 35/SEQ ID NO: 36, wherein in such variant at least the VL or the VH domain is a variant of one of the VL/VH domains listed.

In particular embodiments, said antibody or functional fragment thereof comprises an antigen-binding region that is a variant of the sequences disclosed herein. Accordingly, the invention includes an antibody or a functional fragment thereof having one or more of the properties of the antibody or functional fragment thereof comprising SEQ ID NOs: 1 to 20, particularly the properties defined in Sections [0052] to [0066] and [0068], comprising a heavy chain amino acid sequence with: at least 60 percent sequence identity in the CDR regions with the CDR regions comprised in SEQ ID NO: 2, 4, 6, 8; 10, 12, 14, 16, 18, or 20, particularly SEQ ID NOs: 4, 6, and 10, more particularly SEQ ID NO: 10, particularly at least 70 percent sequence identity, more particularly at least 80 percent sequence identity, and most particularly at least 90 percent sequence identity, and/or at least 80 percent sequence homology, more particularly at least 90 percent sequence homology, most particularly at least 95 percent sequence homology in the CDR regions with the CDR regions comprised in SEQ ID NO: 2, 4, 6, 8; 10, 12, 14, 16, 18, or 20, particularly SEQ ID NOs: 4, 6, and 10, more particularly SEQ ID NO: 10, and/or comprising a light chain amino acid sequence with: at least 60 percent sequence identity in the CDR regions with the CDR regions comprised in SEQ ID NO: 1, 3, 5, 7; 9, 11, 13, 15, 17, or 19, particularly SEQ ID NOs: 3, 5, and 9, more particularly SEQ ID NO: 9, particularly at least 70 percent sequence identity, more particularly at least 80 percent sequence identity, and most particularly at least 90 percent sequence identity, and/or at least 80 percent sequence homology, more particularly at least 90 percent sequence homology, most particularly at least 95 percent sequence homology in the CDR regions with the CDR regions comprised in SEQ ID NO: 1, 3, 5, 7; 9, 11, 13, 15, 17, or 19, particularly SEQ ID NOs: 3, 5, and 9, more particularly SEQ ID NO: 9. Methods for the determination of sequence homologies, for example by using a homology search matrix such as BLOSUM (Henikoff, S. & Henikoff, J. G. (1992). Amino acid substitution matrices from protein blocks. Proc. Natl. Acad. Sci. USA 89, 10915-10919), and methods for the grouping of sequences according to homologies are well known to one of ordinary skill in the art.

In particular embodiments, such a variant comprises a VL sequence comprising the set of CDR1, CDR2 and CDR3 sequences according to the VL sequence of SEQ ID NO: 19, and/or a VH sequence comprising the set of CDR1, CDR2 and CDR3 sequences according to the VH sequence of SEQ ID NO: 20, wherein in each case one of the indicated amino acid residues shown at every degenerate position “X” in SEQ ID NO: 19 and/or 20 is selected. For example, in the case of each of the positions shown as “X(S/N)” in the CDR1 of SEQ ID NO: 19, any such variant comprises either amino acid residue “S” or amino acid residue “N” at the corresponding positions.

In particular other embodiments, such a variant comprises a VL sequence according to the sequence of SEQ ID NO: 19, and/or a VH sequence according to the sequence of SEQ ID NO: 20, wherein in each case one of the indicated amino acid residues shown at every degenerate position “X” in SEQ ID NO: 19 and/or 20 is selected. For example, in the case of the position shown as “X(P/A)” in framework 1 of SEQ ID NO: 19, any such variant comprises either amino acid residue “P” or amino acid residue “A” at that position.

In particular embodiments, said antibody or functional fragment thereof comprises an antigen-binding region which is obtained by humanizing an antigen-binding region of Sections [0085] to [0087], and [0090] to [0092].

In the context of the present invention, said target-binding moiety; and said binding molecule of said multifunctional molecule are not structurally limited so long as they specifically bind to said target and the binding partner of said binding molecule. However, said target-binding moiety; and said binding molecule generally consist of or are formed of one or more oligo- or polypeptides or parts thereof. Particularly, said target-binding moiety; and said binding molecule are antibody-based binding moieties, which typically comprise at least one antibody variable domain or binding fragment thereof.

In particular embodiments of the present invention, said target-binding moiety; and/or said binding molecule are/is an antibody-based binding moieties/moiety, particularly an antibody-based binding moiety comprising a heavy chain variable domain (VH) or binding fragment thereof, more particularly an antibody-based binding moiety comprising a heavy chain variable domain (VH) or binding fragment thereof and a light chain variable domain (VL) or binding fragment thereof. The term “binding fragment”, as used herein, refers to a portion of a given domain, region or part, which is (either alone or in combination with another domain, region or part thereof) still functional, i.e. capable of binding to the first or second antigen recognized by the multifunctional construct.

In particular embodiments, the multifunctional molecule is an antibody format selected from the group consisting of a single-chain diabody (scDb), a tandem scDb (Tandab), a linear dimeric scDb (LD-scDb), a circular dimeric scDb (CD-scDb), a bispecific T-cell engager (BiTE; tandem di-scFv), a disulfide-stabilized Fv fragment (Brinkmann et al., Proc Natl Acad Sci USA. 1993; 90: 7538-7542), a tandem tri-scFv, a tribody, bispecific Fab2, di-miniantibody, tetrabody, scFv-Fc-scFv fusion, di-diabody, DVD-Ig, IgG-scFab, scFab-dsscFv, Fv2-Fc, IgG-scFv fusions, such as bsAb (scFv linked to C-terminus of light chain), Bs1Ab (scFv linked to N-terminus of light chain), Bs2Ab (scFv linked to N-terminus of heavy chain), Bs3Ab (scFv linked to C-terminus of heavy chain), Ts1Ab (scFv linked to N-terminus of both heavy chain and light chain), Ts2Ab (dsscFv linked to C-terminus of heavy chain), and Knob-into-Holes (KiHs) (bispecific IgGs prepared by the KiH technology) and DuoBodies (bispecific IgGs prepared by the DuoBody technology), a VH and a VL domain, each fused to one C-terminus of the two different heavy chains of a KiHs or DuoBody such that one functional Fv domain is formed, Particularly suitable for use herein is a single-chain diabody (scDb), in particular a bispecific monomeric scDb. For reviews discussing and presenting various multifunctional constructs see, for example, Chan Carter, Nature Reviews Immunology 10 (2010) 301-316; Schubert et al., Antibodies 1 (2012) 2-18; Byrne et al., Trends in Biotechnology 31 (2013) 621; Metz et al., Protein Engineering Design & Selection. 2012; 25:571-580).

In a particular embodiment of the present invention, the VH domain of the first and second antibody-based binding moieties of the multifunctional molecule comprises rabbit heavy chain complementarity determining regions (CDRs) grafted onto human heavy chain framework (FW) regions, and the VL domain of the first and second antibody-based binding moieties of the multifunctional molecule comprises rabbit light chain CDRs grafted onto human light chain FW regions.

The heavy chain and light chain CDRs of the first antibody-based binding moiety are particularly derived from a rabbit antibody obtained by immunization of a rabbit with the full-length epsilon chain of human CD3 the full-length. The immunization with the full-length chain of CD3E is suitably conducted by DNA immunization of a rabbit with a plasmid encoding the full-length chain of human CD3E, or, alternatively, with the purified extracellular domain of the epsilon chain of CD3. Further, the heavy chain and light chain CDRs of the second antibody-based binding moiety are particularly derived from a rabbit antibody obtained by immunization of a rabbit with the purified target protein or with a plasmid expressing said target.

The multifunctional constructs of the present invention can be produced using any convenient antibody manufacturing method known in the art (see, e.g., Fischer, N. & Leger, O., Pathobiology 74:3-14 (2007) with regard to the production of multifunctional constructs; and Hornig, N. & Färber-Schwarz, A., Methods Mol. Biol. 907:713-727, 2012 with regard to bispecific diabodies and tandem scFvs). Specific examples of suitable methods for the preparation of the multifunctional construct of the present invention further include, inter alia, the Genmab (Labrijn et al., Proc Natl Acad Sci USA. 2013 Mar. 26; 110(13):5145-50) and Merus (de Kruif et al., Biotechnol Bioeng. 2010 Aug. 1; 106(5):741-50) technologies. Methods for production of multifunctional antibodies comprising a functional antibody Fc part are also known in the art (see, e.g., Zhu et al., Cancer Lett. 86:127-134 (1994)); Suresh et al., Methods Enzymol. 121:210-228 (1986)).

These methods typically involve the generation of monoclonal antibodies, for example by means of fusing myeloma cells with the spleen cells from a mouse that has been immunized with the desired antigen using the hybridoma technology (see, e.g., Yokoyama et al., Curr. Protoc. Immunol. Chapter 2, Unit 2.5, 2006) or by means of recombinant antibody engineering (repertoire cloning or phage display/yeast display) (see, e.g., Chames & Baty, FEMS Microbiol. Letters 189:1-8 (2000)), and the combination of the antigen-binding domains or fragments or parts thereof of two different monoclonal antibodies to give a multifunctional construct using known molecular cloning techniques.

The multifunctional constructs of the present invention are particularly humanized in order to reduce immunogenicity and/or to improve stability. Techniques for humanization of antibodies are well-known in the art. For example, one technique is based on the grafting of complementarity determining regions (CDRs) of a xenogeneic antibody onto the variable light chain VL and variable heavy chain VH of a human acceptor framework (see, e.g., Jones et al., Nature 321:522-525 (1986); and Verhoeyen et al., Science 239:1534-1536 (1988)). In another technique, the framework of a xenogeneic antibody is mutated towards a human framework. In both cases, the retention of the functionality of the antigen-binding portions is essential (Kabat et al., J. Immunol. 147:1709-1719 (1991)).

In particular embodiments, said multifunctional molecule is a bispecific scDb comprising two variable heavy chain domains (VH) or fragments thereof and two variable light chain domains (VL) or fragments thereof connected by linkers L1, L2 and L3 in the order V_{H}A-L1-V_{L}B-L2-V_{H}B-L3-V_{L}A, V_{H}A-L1-V_{H}B-L2-V_{L}B-L3-V_{L}A, V_{L}A-L1-V_{L}B-L2-V_{H}B-L3-V_{H}A, V_{L}A-L1-V_{H}B-L2-V_{L}B-L3-V_{H}A, V_{H}B-L1-V_{L}A-L2-V_{H}A-L3-V_{L}B, V_{H}B-L1-V_{H}A-L2-V_{L}A-L3-V_{L}B, V_{L}B-L1-V_{L}A-L2-V_{H}A-L3-V_{H}B or V_{L}B-L1-V_{H}A-L2-V_{L}A-L3-V_{H}B, particularly VLB-L1-V_{H}A-L2-V_{L}A-L3-V_{H}B, wherein the V_{L}A and V_{H}A domains jointly form the antigen binding site for the first antigen, and V_{L}B and V_{H}B jointly form the antigen binding site for the second antigen, particularly wherein linker L1 is a peptide of 2-10 amino acids, particularly 3-7 amino acids, particularly 5 amino acids, particularly GGGGS, linker L3 is a peptide of 1-10 amino acids, particularly 2-7 amino acids, particularly 5 amino acids, particularly GGGGS, and the linker L2 is a peptide of 10-40 amino acids, particularly 15 to 30 amino acids, particularly 20 to 25 amino acids, particularly 20 amino acids, particularly (GGGGS)₄.

The multifunctional molecule of the present invention may alternatively comprise one or more binding moieties based on non-antibody based binding domains. Specific examples of suitable methods for the preparation of the multifunctional construct of the present invention further include, inter alia, the DARPin technology (Molecular Partners AG), the adnexin technology (Adnexus), the anticalin technology (Pieris), and the Fynomer technology (Covagen AG).

In a third aspect, the present invention relates to a multifunctional molecule comprising at least (i) a target-binding moiety; and (ii) a binding molecule, which is a binding molecule, particularly an antibody or a functional fragment thereof, binding to essentially the same epitope as the antibody or functional fragment thereof of Sections [0085] to [0087], [0090] to [0092] and [0096].

In particular embodiments, said antibody or functional fragment thereof is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, said epitope comprises amino acid residue N4 as residue that is critical for binding, particularly wherein said epitope further comprises amino acid residue E6 as residue that is involved in binding; and/or wherein at least one of residues Q1, D2, G3 and E5 of human CD3e is non-critical for binding.

In the context of the present invention, an amino acid residue is to be considered “critical for binding”, when the binding affinity of a binding molecule to a peptide comprising said amino acid residue position is reduced to at least 50%, particularly to at least 25%, more particularly to at least 10%, and most particularly to at least 5% of the binding affinity to the wild-type peptide sequence, when said critical amino acid residue is exchanged by alanine. and/or when the average signal intensity resulting from binding to a peptide comprising said amino acid residue position as determined by the ELISA of Example 7 is reduced to at least 50%, particularly to at least 25%, and most particularly to at least 10% of the binding signal to the wild-type peptide sequence, when said critical amino acid residue is separately exchanged by each of the other natural amino acid residues except cysteine.

In particular embodiments, said epitope further comprises amino acid residue E6 as residue that is involved in binding. In particular embodiments, said epitope further comprises amino acid residue E6 as residue that is critical for binding.

In the context of the present invention, an amino acid residue is to be considered “involved in binding”, when the binding affinity of a binding molecule is reduced to at least 80%, when said amino acid residue is exchanged by alanine, and/or when the average signal intensity resulting from binding to a peptide comprising said amino acid residue position as determined by the ELISA of Example 7 is reduced to at least 80%, when said amino acid residue is separately exchanged by each of the other natural amino acid residues except cysteine.

In particular embodiments, at least one of residues Q1, D2, G3 and E5 of CD3e is non-critical for binding. In particular embodiments, at least two of residues Q1, D2, G3 and E5 of CD3e is non-critical for binding, more particularly at least three, and most particularly all four residues Q1, D2, G3 and E5 of CD3e are non-critical for binding.

In the context of the present invention, an amino acid residue is to be considered “non-critical for binding”, when the binding affinity of a binding molecule to a peptide comprising said amino acid residue position is reduced to not less 80%, more particularly to not less than 90%, and most particularly to not less than 95% of the binding affinity to the wild-type peptide sequence, when said non-critical amino acid residue is exchanged by alanine. and/or when the average signal intensity resulting from binding to a peptide comprising said amino acid residue position as determined by the ELISA of Example 7 is reduced to not less than 50%, particularly to not less than 70%, more particularly to not less than 80%, and most particularly to not less than 90% of the binding signal to the wild-type peptide sequence, when said non-critical amino acid residue is separately exchanged by each of the other natural amino acid residues except cysteine.

In particular embodiments, said binding region is an antibody or a functional fragment thereof comprising an antigen-binding region comprising a VL domain selected from the group of SEQ ID NOs: 21, 23, and 24, and the VH domain of SEQ ID NO: 22.

In particular embodiments, said target-binding moiety is an antibody or a functional fragment thereof comprising an antigen-binding region comprising a VL domain selected from the group of SEQ ID NOs: 25, 26, and 27, and the VH domain of SEQ ID NO: 28 or an antigen-binding region comprising the VL domain of SEQ ID NO: 33, and the VH domain of SEQ ID NO: 34.

In particular embodiments, said multifunctional molecule comprises the single-chain fragment of SEQ ID NO: 37.

In alternative embodiments, said second functional moiety specifically binds to an Fc receptor, in particular to an Fc gamma receptor (FcγR), in particular to (i) an FcγRIII present on the surface of natural killer (NK) cells or (ii) one of FcγRI, FcγRIIA, FcγRIIB1, FcγRIIB2, and FcγRIIIB present on the surface of macrophages, monocytes, neutrophils and/or dendritic cells.

In such embodiment, the first binding moiety particularly is an Fc region or functional fragment thereof. In the present context, a “functional fragment” refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. Particularly, a functional Fc fragment is capable of competitively inhibiting the binding of the original, full-length Fc portion to an FcR such as the activating FcγRI. Particularly, a functional Fc fragment retains at least 30%, 40%, 50%, 60%, 70%, 80%, 90% or 95% of its affinity to an activating FcγR.

Within such embodiment of the present invention, the Fc region or functional fragment thereof is particularly an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g. FcγRIIIA (CD16A) expressed on natural killer (NK) cells) and/or a decreased binding to an inhibitory receptor (e.g. FcγRIIB1/B2 (CD32B)).

Suitable alterations within the present invention include altering glycosylation patterns, in particular afucosylation (also referred to as “defucosylation”), mutations (point mutations, deletions, insertions) and fusions with oligo- or polypeptides. Known techniques for altering glycosylation patterns include overexpression of heterologous β1,4-N-acetylglucosaminyltransferase III in the antibody-producing cell (known as the Glycart-Roche technology) and knocking out of the gene encoding α-1,6-fucosyltransferase (FUT8) in the antibody-producing cell (the Potelligent technology from Kyowa Hakko Kirin). Specific examples of enhancing mutations in the Fc part include those described in Shields et al., J. Biol. Chem. 276:6591-6604 (2001), which is incorporated herein in its entirety to the extent possible under the respective patent laws and regulations.

In particular embodiments, said patient does not respond to treatment with antagonists of cytokines that are involved in the differentiation of (i) T cells expressing the transcription factor RORγ(t), (ii) T cells producing GM-CSF and/or IFN gamma, and/or IL-17, particularly an IL-17 producing T cells (Th17 cells), (iii) γδ T cells, (iv) a natural killer T (NKT) cells, and (v) invariant natural killer (iNK) cells; particularly a Th17 cells or a γδ T cells.

In particular embodiments, said patient does not respond to treatment with IL-23 antagonists.

In the context of the present invention, the term “IL-23 antagonists” refers to molecules that directly or indirectly interfere with IL-23-mediated signaling, for example by interfering with binding of IL-23 to IL23R and/or IL12Rβ1 transmembrane proteins, respectively, or by interfering with the expression of IL23: Thus the term “IL-23 antagonists” includes molecules binding to IL-23 by binding to p19 and/or p40 of IL-23, such as antibodies or antibody fragments such as ustekinumab (Longbrake et al., loc. cit), soluble fragments of the IL23R, such as Δ9 (see Yu and Gallagher, J Immunol. 85 (2010) 7302-8), and IL23R-derived antagonistic peptides (see, for example, Quiniou et al., Am J Physiol Regul Integr Comp Physiol. 307 (2014) R1216-30).

In particular embodiments, said exacerbation episode is a clinically isolated syndrome.

In the context of the present invention, the term “clinically isolated syndrome” refers to a first episode of neurologic symptoms that lasts at least 24 hours and is caused by inflammation and demyelination in one or more sites in the central nervous system (CNS). CIS can be either monofocal or multifocal. In a monofocal episode, the person experiences a single neurologic sign or symptom—for example, an attack of optic neuritis—that's caused by a single lesion. In a multifocal episode, the person experiences more than one sign or symptom—for example, an attack of optic neuritis accompanied by weakness on one side—caused by lesions in more than one place.

In a third aspect, the present invention relates to a multifunctional molecule for use in the prophylactic treatment of multiple sclerosis to prevent or delay an exacerbation episode, wherein said multifunctional molecule comprises at least (i) a target-binding moiety, which is specific for IL23R; and (ii) a second functional moiety, which leads to the depletion of IL23R-expressing cells.

In particular embodiments of the aspects of the present invention, said multiple sclerosis is a progressive form of multiple sclerosis, in particular a progressive form of multiple sclerosis accompanied by systemic inflammation.

In the context of the present invention, the term “progressive form of multiple sclerosis” refers to any of the two progressive forms of MS, in particular, primary progressive (PP)-MS and secondary progressive (SP)-MS. PPMS is characterized by steady worsening of neurologic functioning, without any distinct relapses (also called attacks or exacerbations) or periods of remission. A person's rate of progression may vary over time—with occasional plateaus or temporary improvement—but the progression is continuous. In contrast, SPMS follows after the relapsing-remitting disease course (RRMS). Of the 85 percent of people who are initially diagnosed with RRMS, most will eventually transition to SPMS, which means that after a period of time in which they experience relapses and remissions, the disease will begin to progress more steadily (although not necessarily more quickly), with or without any relapses (also called attacks or exacerbations).

In the context of the present invention, the term “systemic inflammation” refers to an inflammation established outside of the CNS, particularly a condition resulting from the release of pro-inflammatory cytokines from immune-related cells and the chronic activation of the innate immune system.

In particular embodiments, said exacerbation episode is an acute phase of a neuromyelitis optica.

In the context of the present invention, the term “neuromyelitis optica” refers to the combined demyelination of the optic nerve and spinal cord, with diminution of vision and possible blindness, flaccid paralysis of extremities, and sensory and genitourinary disturbances. Neuromyelitis optica is also known as Devic's disease.

In particular embodiments, said exacerbation episode is an acute phase of Asian multiple sclerosis.

In the context of the present invention, the term “Asian multiple sclerosis” refers to a variant of neuromyelitis optica that can present brain lesions like MS.

### EXAMPLES

The following examples illustrate the invention without limiting its scope.

### Example 1: Identification and Selection of Monoclonal Antibodies Binding to a T Cell-Stimulatory Epitope on CD3

Rabbit memory B cells binding to CD3ε were isolated from one immunized rabbit using fluorescence activated cell sorting. In order to exclude antibodies binding to the interface of CD3ε and CD3γ, a Phycoerythrin (PE)-labeled single-chain protein construct was used consisting of the extracellular domains of CD3ε and CD3γ joined by a flexible peptide linker (scCD3γε). In total, 4,270 memory B cells binding to PE-scCD3γε were individually sorted into 96-well culture plates and cultured at conditions published elsewhere (Lightwood et al, JIM 2006; 316: 133-143). All culture supernatants were first screened by ELISA for binding to scCD3γε, which yielded 441 hits. In a second screening, positive supernatants from the first screening were tested for their ability to bind the native CD3ε embedded in the TCR complex on the surface of Jurkat cells (see Methods below). A total of 22 hits showed binding to CD3ε expressing Jurkat cells but not to cd3−/− Jurkat cells. The affinity to the purified extracellular domain of heterodimeric CD3εγ from human and cynomolgus monkey origin was measured using SPR for the 22 hits. Affinities to human CD3εγ as expressed by K_{D} ranged from 0.16 to 9.28 nM (data not shown). One of the screening hits did not show binding by SPR and was therefore not considered for further analysis.

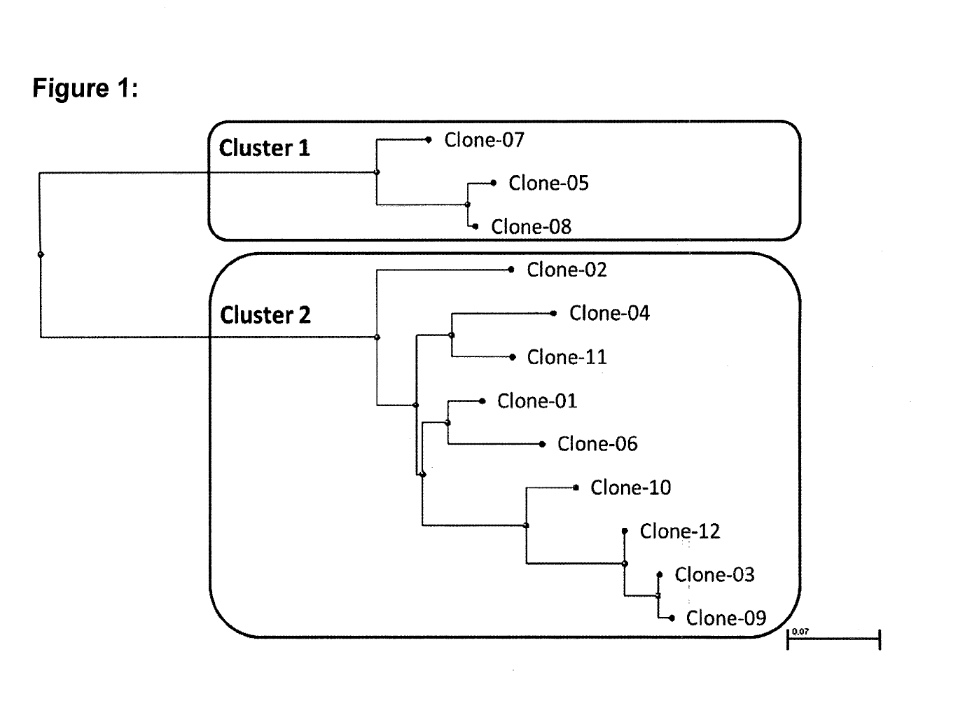

The DNA sequence encoding the variable domains of the remaining 21 clones were retrieved by RT-PCR and DNA sequencing and resulted in 18 independent clones. These rabbit IgGs were recombinantly produced in a mammalian expression system and were characterized in terms of affinity to scCD3γε from human and cynomolgus origin and their ability to bind to Jurkat cells. Phylogenetic sequence analysis of these 18 sequences revealed two main clusters, which clearly differed from each other, while there was significant homology within the two clusters (FIG. 1). As all representatives from one cluster presumably derive from the same antigen-binding parent B cell they likely bind to the same epitope. Thus, in order to cover the maximal diversity, the most diverse clones were selected from each cluster resulting in 12 clones that were further tested for their ability to bind and activate T cells. T cell binding was assessed in a cell-based ELISA and T cell stimulation was quantified by measuring expression of CD69 by FACS. Representative antibodies were further characterized as shown in Examples 2 to 4.

### Example 2: Binding of Purified Monoclonal Rabbit Anti-CD3ε Antibodies to Jurkat T Cells and to Cynomolgus Monkey HSC-F T Cells

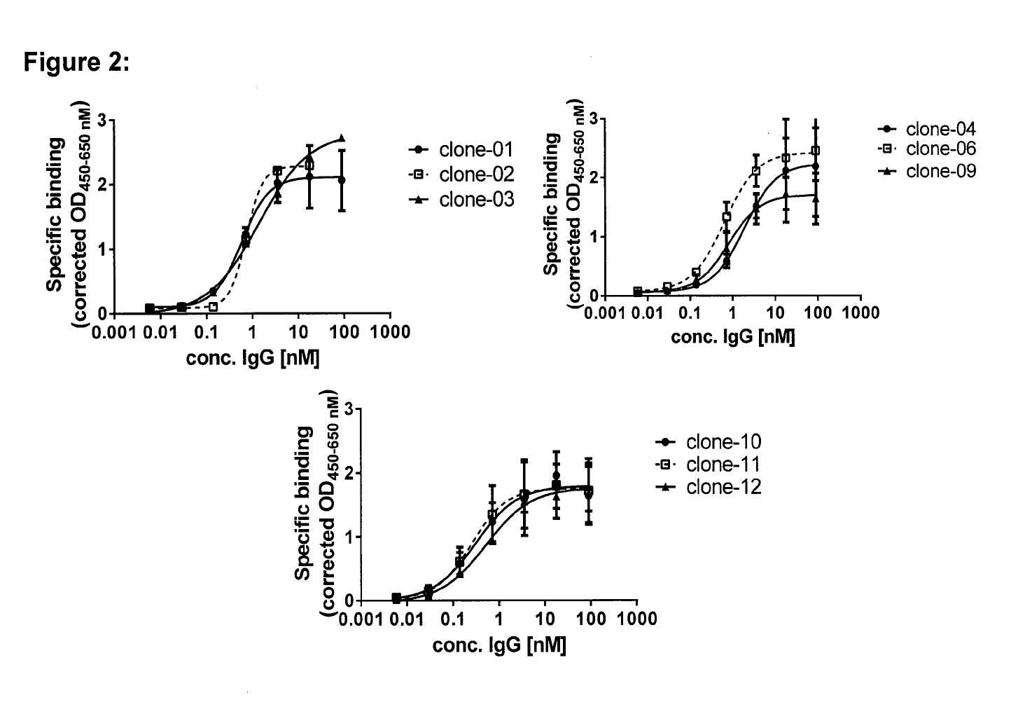

Jurkat human T cells and cynomolgus monkey HSC-F T cells were incubated with increasing concentrations of the purified monoclonal rabbit antibodies, as described in the methods section. With all antibodies tested, specific binding to human CD3ε increased with increasing antibody concentrations (FIG. 2). The EC₅₀ values, indicating half-maximal binding to Jurkat human T cells, were very similar for all antibodies, ranging from 0.28 to 1.87 nM (see Table 1, which shows the pharmacodynamic characteristics of purified monoclonal rabbit antibodies. For the qualitative detection of CD69 expression the mean fluorescence intensity (MFI), reflecting the signal intensity at the geometric mean, was measured for both, the negative control as well as for the test antibodies. The normalized MFI was calculated by dividing the MFI of the test antibody through the MFI of the negative control antibody.). EC₅₀ values for binding to cynomolgus monkey HSC-F T cells are shown for three antibodies (clone-06, clone-02, clone-03) (see Table 2C).

TABLE 2A

| Clone ID | KD (human)/KD (cyno) |
|---|---|
| clone-01 | 3 |
| clone-02 | 13 |
| clone-03 | 13 |
| clone-04 | 36 |
| clone-06 | 4 |
| clone-09 | 10 |
| clone-10 | 7 |
| clone-11 | 3 |
| clone-12 | 20 |

TABLE 2B

| | Affinity to human CD3ε | Affinity to cyno CD3ε |
|---|---|---|
| Clone ID | [KD] | [KD] |
| clone-06 | 2.23 × 10⁻⁹ M | 8.95 × 10⁻⁹ M |
| clone-02 | 3.04 × 10⁻¹⁰ M | 3.86 × 10⁻⁹ M |
| clone-03 | 9.05 × 10⁻¹⁰ M | 1.15 × 10⁻⁸ M |

TABLE 2C
Rabbit IgG binding to cell surface

| | Binding to human Jurkat | Binding to cyno HSC-F |
|---|---|---|
| Clone ID | T cells [EC₅₀] | T cells [EC₅₀] |
| clone-06 | 0.67 nM | 1.6 nM |
| clone-02 | 0.71 nM | 3.82 nM |
| clone-03 | 1.45 nM | 23.9 nM |

TABLE 1

| | | | | | | | | | Fold increase in | | |
|---|---|---|---|---|---|---|---|---|---|---|---|
| | | | | | | | | | CD69 expression: | | |
| | | | | | | | Specific binding to | | [MFI normalized | | |
| | | | | | | | Jurkat cells | | to neg. ctrl.] | | |
| | SPR data human CD3ge | | | SPR data cyno CD3ge | | | | relative EC₅₀ | 20 μg/ml | 5 μg/ml | 1.25 μg/ml |
| | ka [M⁻¹ | | KD | ka [M⁻¹ | | KD | | (EC_{50,clone 5}/ | anti-CD3 | anti-CD3 | anti-CD3 |
| Clone ID | s⁻¹] | kd [s⁻¹] | [M] | s⁻¹] | kd [s⁻¹] | [M] | EC50 (nM) | EC_{50,test}) | IgG | IgG | IgG |
| clone-01 | 5.36E+05 | 1.59E−03 | 2.97E−09 | 3.86E+05 | 3.92E−03 | 1.02E−08 | 0.58 | 0.88 | ND | ND | ND |
| clone-02 | 8.69E+05 | 2.64E−04 | 3.04E−10 | 6.68E+05 | 2.58E−03 | 3.86E−09 | 0.71 | 0.59 | 7.4 | 4.6 | 3.3 |
| clone-03 | 5.51E+05 | 4.98E−04 | 9.05E−10 | 3.50E+05 | 4.03E−03 | 1.15E−08 | 1.45 | 0.37 | 6.6 | 4.6 | 2.6 |
| clone-04 | 8.73E+05 | 9.88E−05 | 1.13E−10 | 6.46E+05 | 2.66E−03 | 4.12E−09 | 1.87 | 0.29 | 7.8 | 3.5 | 2.6 |
| clone-06 | 6.18E+05 | 1.38E−03 | 2.23E−09 | 4.44E+05 | 3.97E−03 | 8.95E−09 | 0.67 | 0.76 | 5.3 | 5.1 | 2.7 |
| clone-09 | 6.01E+05 | 6.88E−04 | 1.14E−09 | 2.32E+05 | 2.69E−03 | 1.16E−08 | 0.82 | 0.90 | ND | ND | ND |
| clone-10 | 7.57E+05 | 1.26E−03 | 1.66E−09 | 3.21E+05 | 3.49E−03 | 1.09E−08 | 0.35 | 2.10 | 6.2 | 4.2 | 2.6 |
| clone-11 | 4.25E+05 | 1.33E−03 | 3.13E−09 | 3.63E+05 | 3.65E−03 | 1.00E−08 | 0.28 | 2.39 | ND | ND | ND |
| clone-12 | 7.21E+05 | 7.98E−04 | 1.11E−09 | 1.42E+05 | 3.14E−03 | 2.22E−08 | 0.59 | 1.14 | ND | ND | ND |

| | | | | | | |
|---|---|---|---|---|---|---|
| OKT3 | ND | ND | ND | 3.1 | 2.5 | 1.8 |
| TR66 | ND | ND | ND | 3.0 | 2.2 | 1.6 |

### Example 3: Potential of Purified Monoclonal Rabbit Anti-CD3ε Antibodies to Stimulate CD69 Expression on T Cells

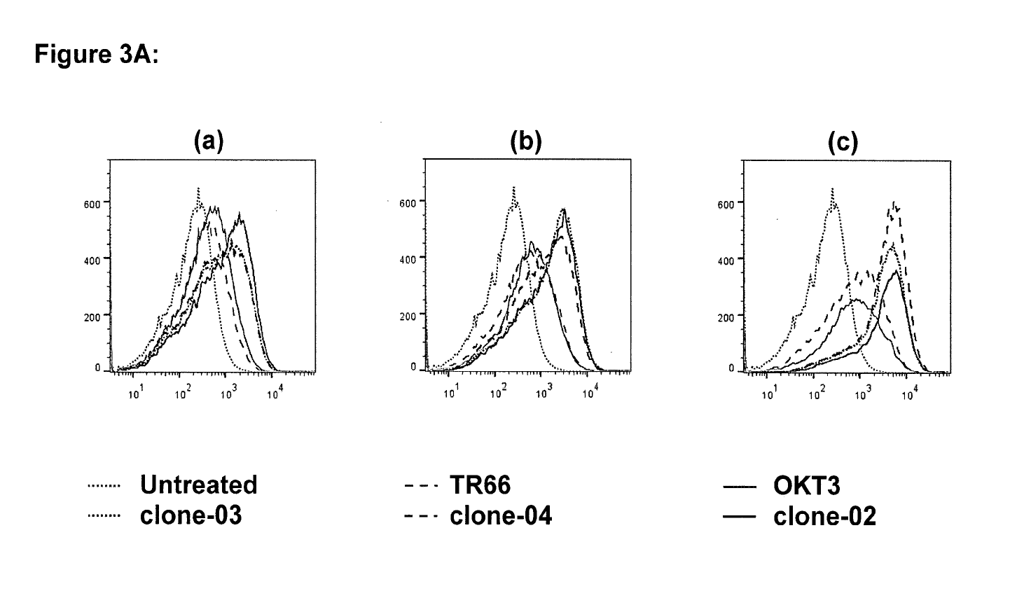

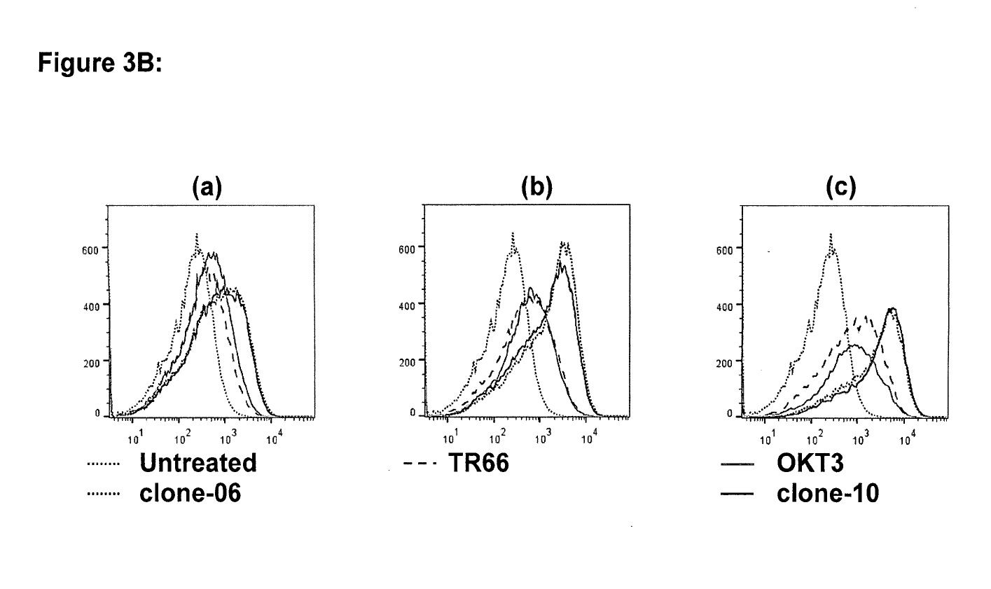

The potential of purified monoclonal rabbit anti-CD3 antibodies to induce T-cell activation as assessed by measurement of CD69 expression (see methods) was compared to the published antibodies OKT-3 and TR66. In the first approach, three different concentrations of cross-linked antibodies were used to stimulate Jurkat cells and CD69 expression was assessed by flow-cytometry 24 h later. A significant increase in CD69 expression was observed with all tested antibodies at 1.25 μg/ml (FIG. 3 and Table 1). Interestingly, all tested rabbit antibodies showed stronger stimulation of CD69 expression than the published OKT-3 and TR66. This is unexpected as the rabbit antibodies bind to human scCD3γε with much higher affinity than OKT-3 or TR66, which, according to prior art, should negatively affect their ability to serially trigger and thereby enhance TCR signaling. With increasing concentrations of rabbit antibodies the CD69 expression level further increased, while there was only a moderate increase in CD69 expression with increasing concentrations of OKT-3 or TR66. Further with the rabbit antibodies, the peak in the histogram became narrower indicating a more homogenous population of T cells, all expressing CD69 at similarly high levels. In contrast there were broad distributions of CD69 expression levels in the T cell populations stimulated with OKT-3 or TR66 at each concentration tested. An antibody that leads to distinct and homogenous T cell activation levels depending on the dose allows for better dose adjustment to optimize efficacy and to control side effects.

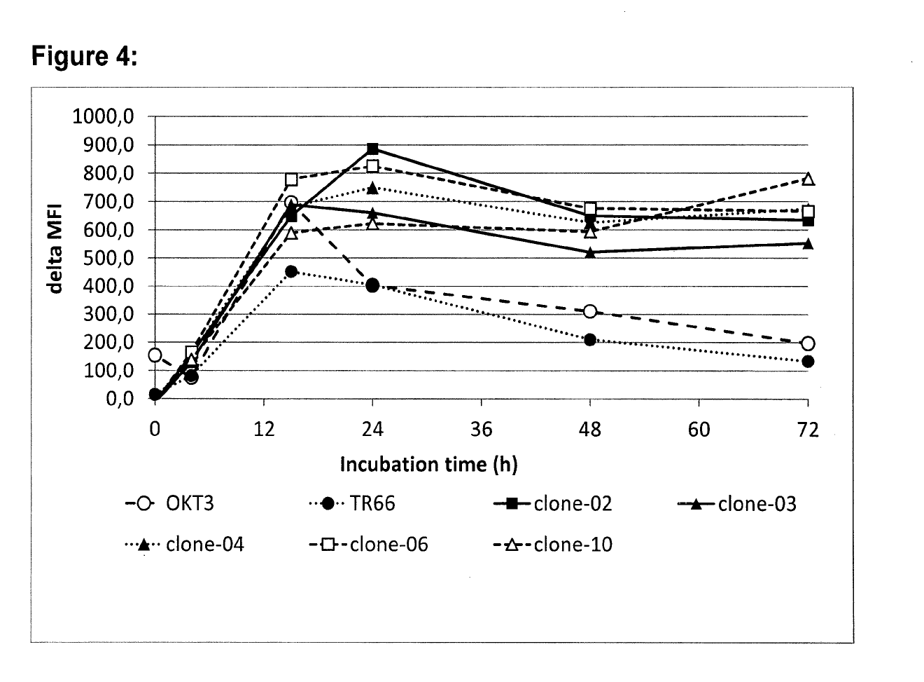

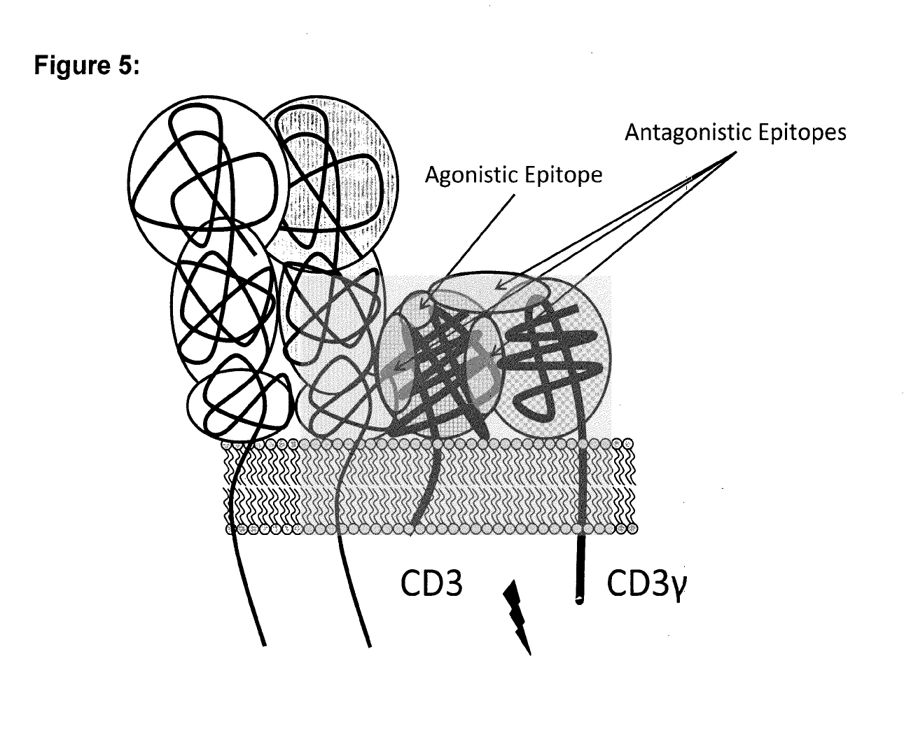

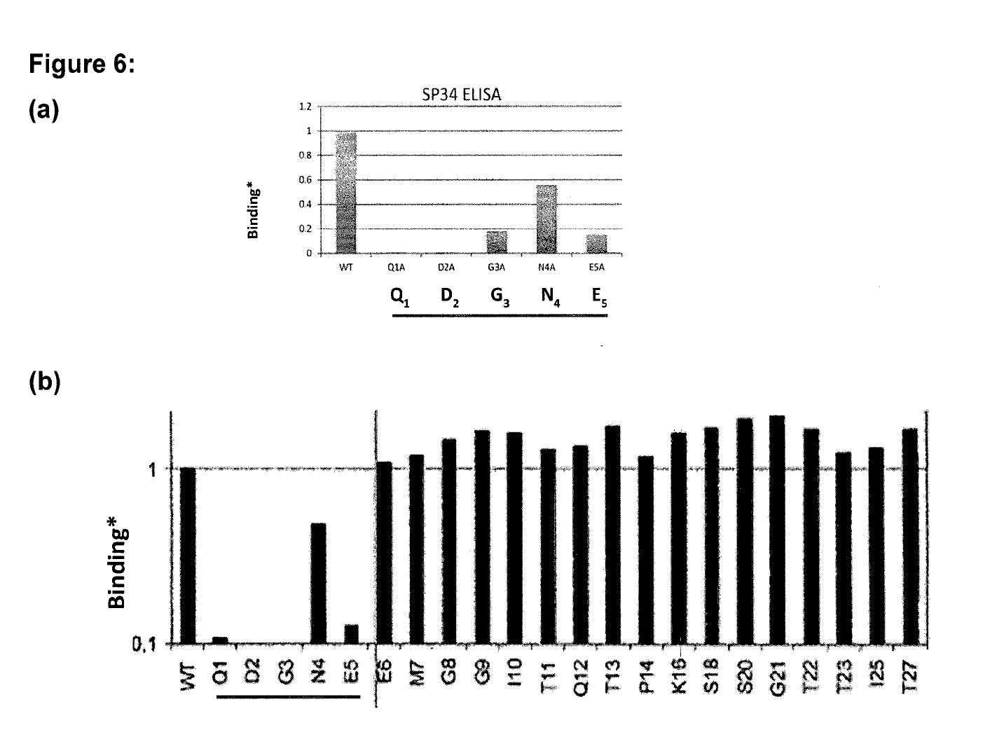

In the second approach, T-cell activation after different time points of stimulation by anti-CD3 antibodies was analyzed. Jurkat cells were stimulated by cross-linked antibodies and CD69 expression was assessed as described above after 0, 4, 15, 24, 48 and 72 h (FIG. 4).

### Example 4: Binding of Anti-CD3×Anti-IL5R Antibodies to Jurkat T Cells and CHO-IL5R Cells

In order to show the benefit of the agonistic anti-CD3 antibodies, a set of bispecific anti-CD3×IL5R single-chain diabodies (scDbs) were constructed by standard methods (methods/data not shown; Construct 1=comprises the humanized variable domain of clone-06; Construct 2=comprises the humanized variable domain of clone-02; Construct 3=comprises the humanized variable domain of clone-03).

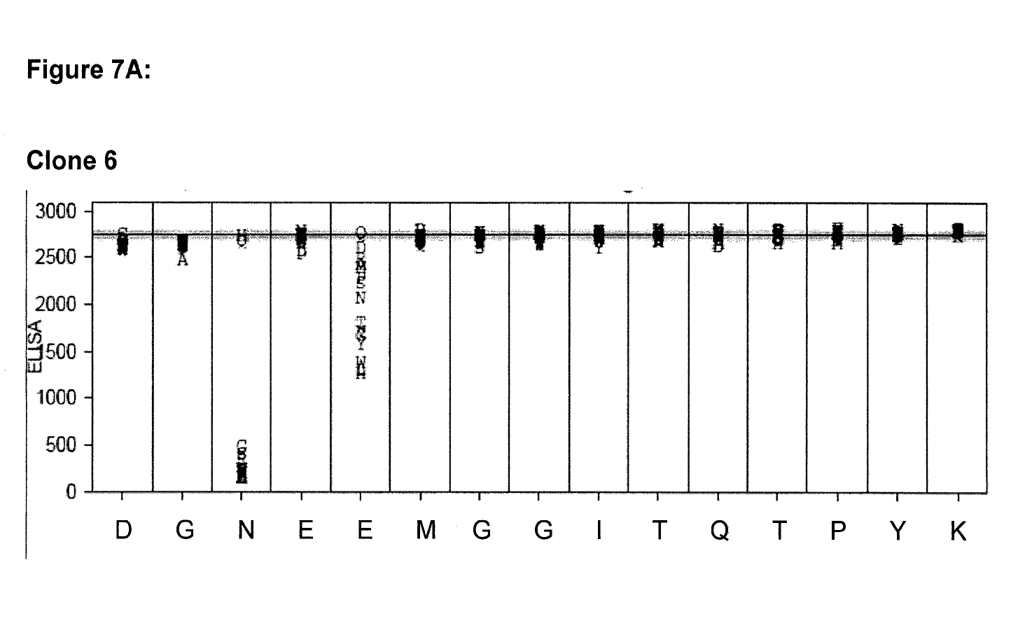

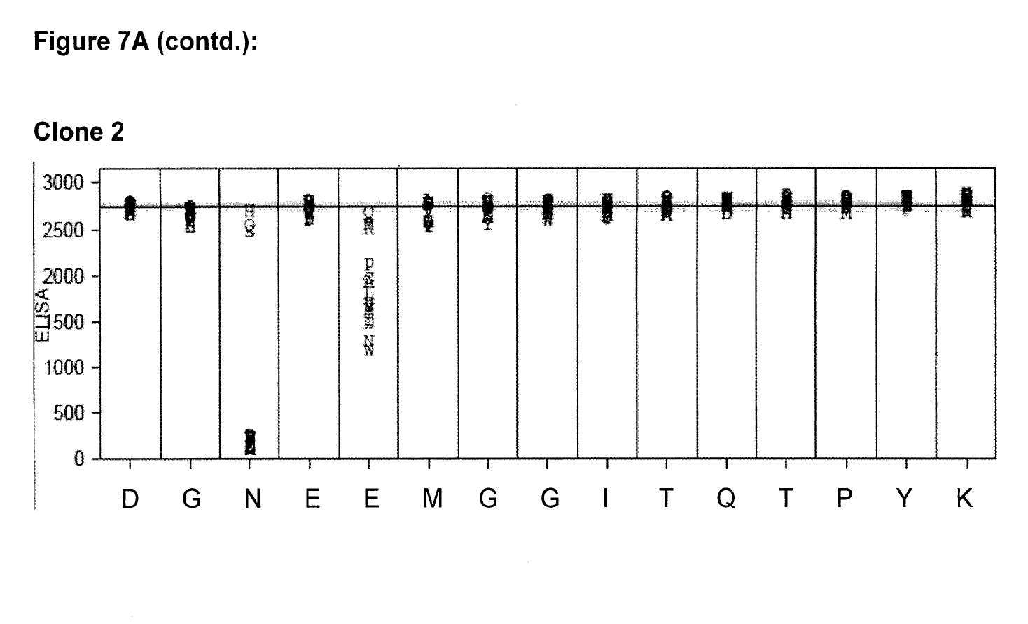

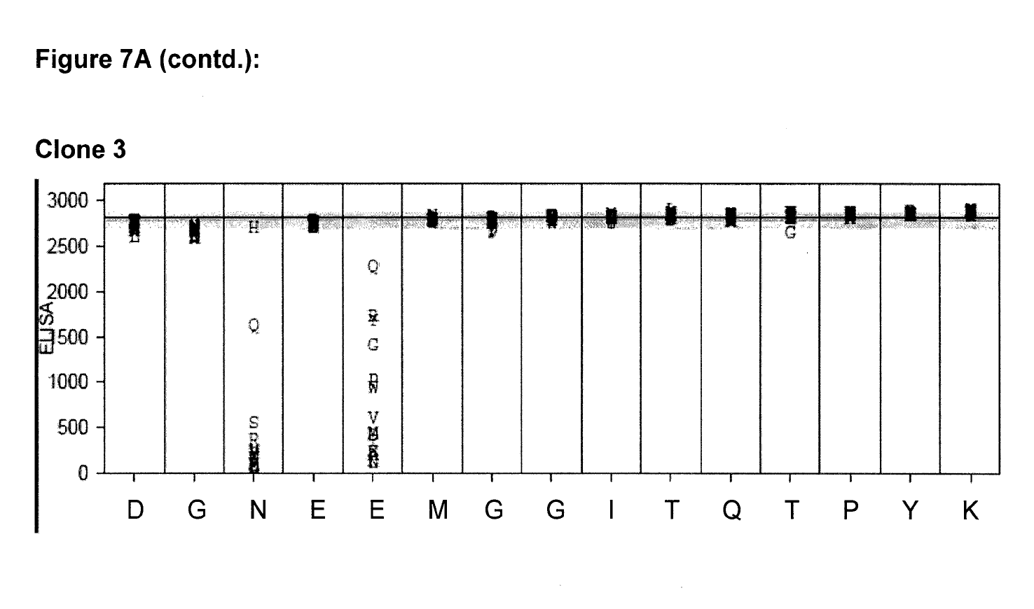

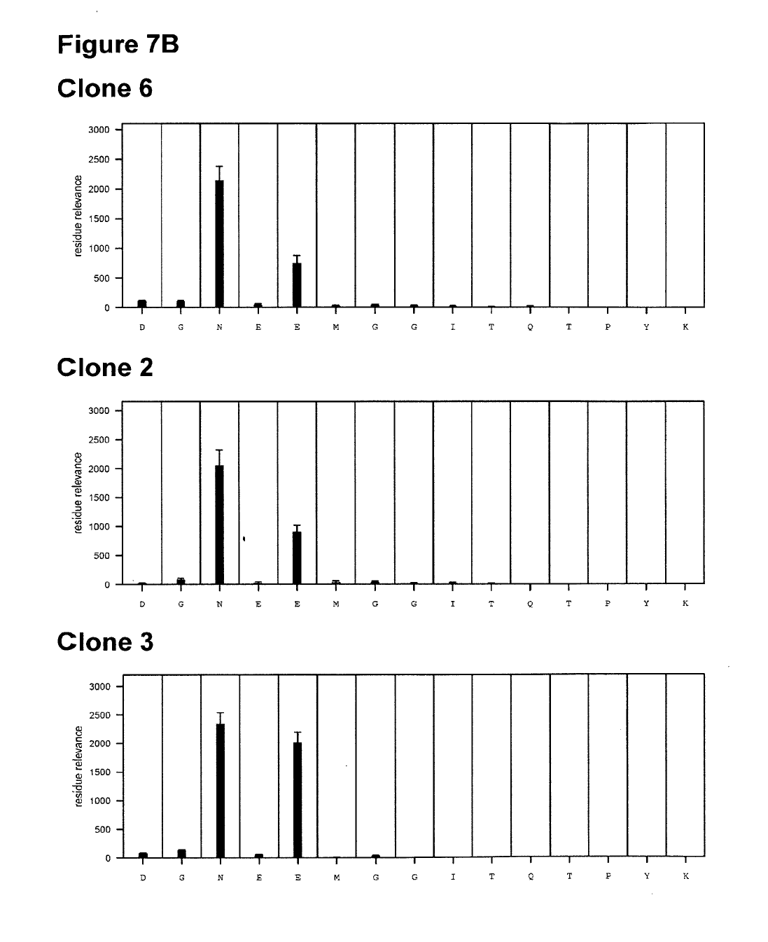

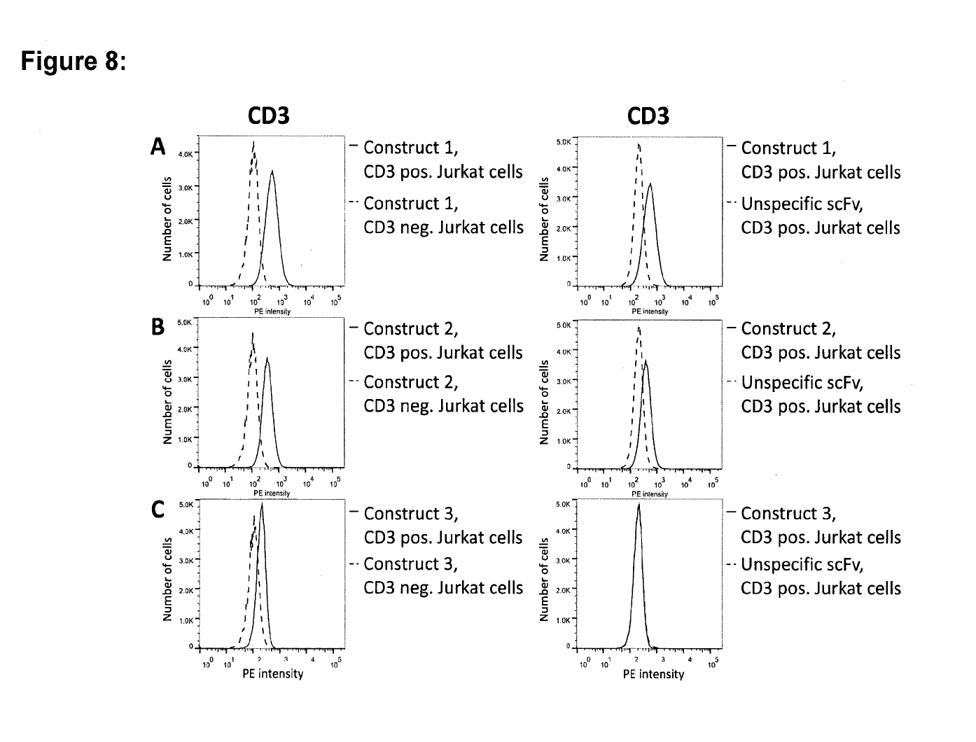

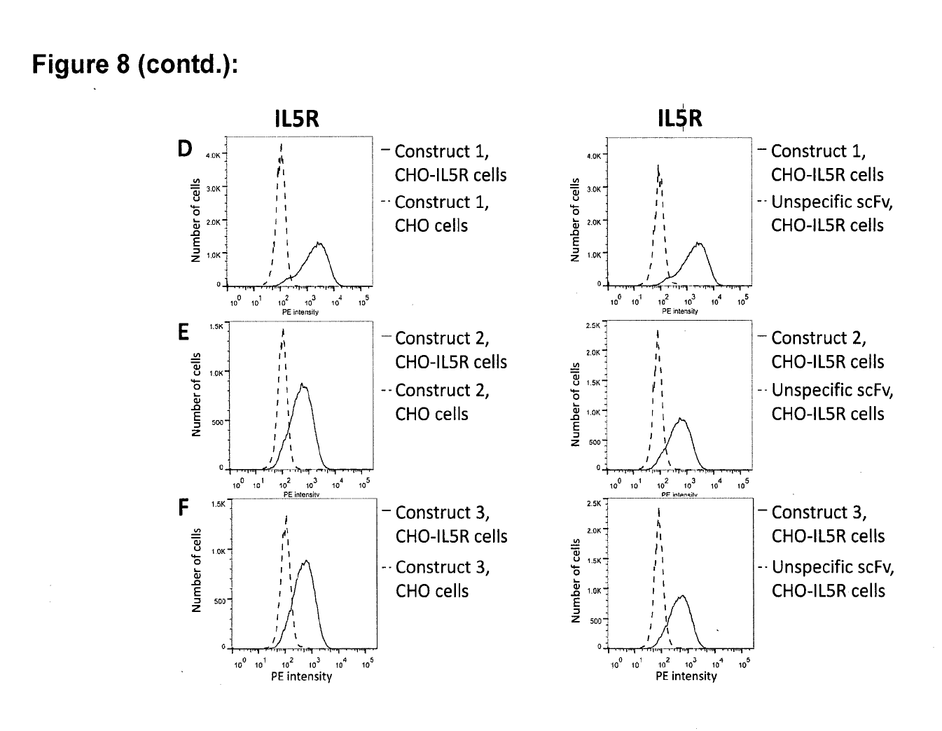

Jurkat T cells and IL5R-expressing CHO cells (CHO-IL5R) are incubated with 1 μg/ml and 10 μg/ml of the scDbs, as described in the methods section. With all scDbs tested, specific binding to CD3ε and IL5R expressing cell lines but no unspecific binding to control cell lines is detected. The three different scDbs (Constructs 1 to 3) containing the identical anti-IL5R moiety while the anti-CD3 moieties being different, were tested for specific binding to cells expressing either IL5R or CD3ε. The anti-CD3 parts bind to overlapping epitopes with variable affinities though (Table 1 and 3 and FIG. 7). As expected the binding to CHO-IL5R cells was similar for all scDbs tested (FIG. 8). In contrast, binding to Jurkat T-cells decreased with decreasing affinity of the CD3ε binding domain. No binding to Jurkat T-cells was detected for the low affinity binder Construct 3 at the highest concentration tested (FIG. 8).

### Example 5: Potential of Bispecific Anti-CD3×IL5R scDbs to Stimulate IL-2 Secretion from T Cells

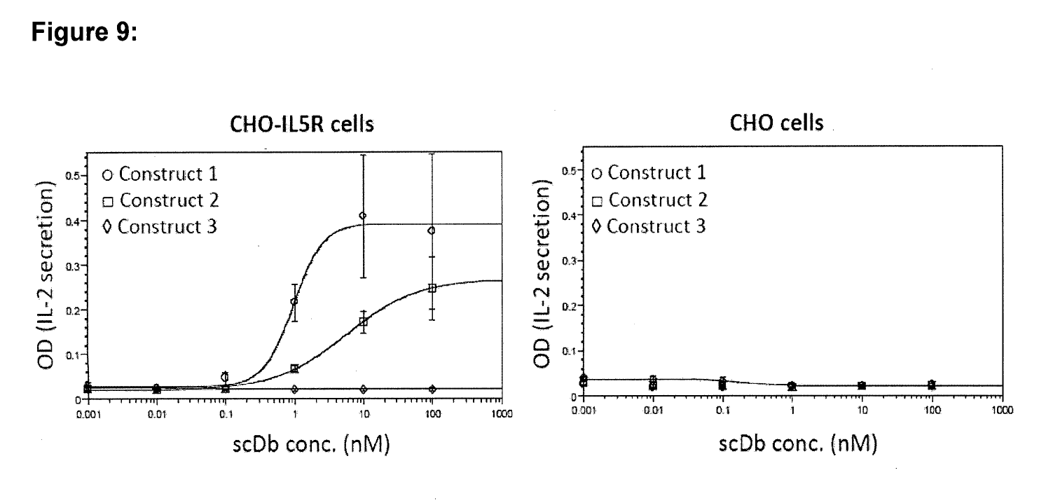

The potential of scDbs bound to a target cell to induce T-cell activation can be assessed by measurement of IL-2 secretion (see methods) by cytotoxic T-cells purified from human blood. The different scDbs are incubated with CD8+ cytotoxic T-cells in presence of target expressing CHO-IL5R cells at an effector:target cell ratio of 10:1 and IL-2 secretion is analyzed after 16 hours of incubation. A dose-dependent stimulation of IL-2 secretion is observed in presence of CHO-IL5R cells while essentially no IL-2 secretion is observed in presence of wild-type CHO cells (see representative data in Table 3 and in FIG. 9). Therefore, T-cell activation is specifically induced in presence of target expressing cells. Moreover, the potential to induce IL-2 secretion correlates with binding affinity to recombinantly produced CD3εγ and to the capacity to bind to T-cells. In line with affinity analysis, Construct 1, which is the binder with the highest affinity, is a more potent inducer of IL-2 secretion than Construct 2, while no IL-2 secretion is observed with the low affinity scDb Construct 3 (FIG. 9).

TABLE 3
Humanized anti-CD3ε domains in the IL5R × CD3 scDb format

| | | | | Potency to |
|---|---|---|---|---|
| | Affinity human | Potency to lyse | | stimulate IL-2 |
| | CD3ε | target cells | | secretion by T cells |
| Clone ID | [KD] | [EC₅₀] | | [EC₅₀] |
| clone-06 | 1.15 × 10⁻⁸ M | 0.1 | nM | 0.96 nM |
| clone-02 | 2.96 × 10⁻⁸ M | 0.96 | M | 5.67 nM |

| | | | |
|---|---|---|---|
| clone-03 | 1.23 × 10⁻⁷ M | no lysis | no signal |

### Example 6: Specific scDb Mediated Target Cell Lysis by Cytotoxic T-Cells

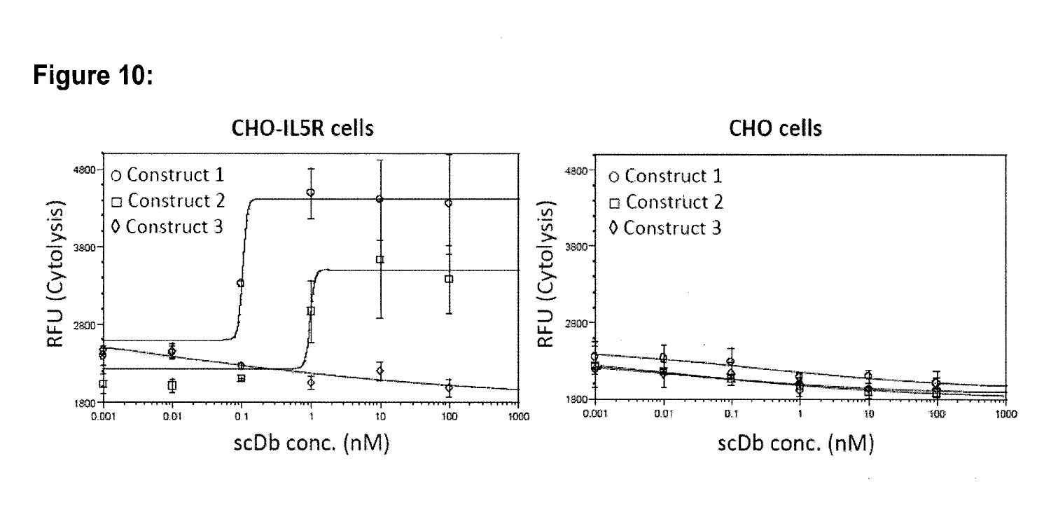

Specific lysis of target cells by cytotoxic T-cells mediated by anti-CD3×IL5R scDbs is analyzed with the CellTox™ green cytotoxicity assay (see methods) after 88 hours of incubation. Similarly to results discussed above for T-cell activation, a dose-dependent target cell lysis is observed for Construct 1 and Construct 2 in presence of CHO-IL5R cells while no lysis is observed in presence of wild-type CHO cells (see representative data for constructs 1 to 3 in Table 3 and in FIG. 10). In line with results mentioned above, scDbs binding with high affinity to CD3ε shows more potent lysis compared to the lower affinity scDbs. No target cell lysis is observed for the low affinity scDb Construct 3. We further tested the potency to lyse target cells of a scDb containing the humanized variant of an additional CD3ε binding clone (clone-05) originating from a different cluster (cluster 1) of antibodies that are also cross-reactive to cynomolgus monkey CD3ε. Clone-05 binds with even higher affinity (KD=8.45×10⁻¹⁰ M and 4.29×10⁻⁹ M for the rabbit IgG and humanized derivative thereof, respectively) as compared to clone-06, but importantly, binds to a different epitope than the binders from cluster 2 (clone-06, clone-02 and clone-03). Interestingly, we found that the respective anti-IL5R×CD3 scDb showed weaker potency to induce T-cell dependent lysis of CHO-IL5R target cells (EC₅₀=9.9×10⁻⁹ M) than the scDb containing humanized clone-06, suggesting that the epitope of clone-06 is particularly well suited for the redirection of cytotoxic T cells to lyse target cells. The superior potency of the cross-linked parental IgGs from cluster 2 versus OKT-3 and TR66 (example 3) confirm that even with higher affinities than those tested in the scDb format no affinity optimum was found after which the potency would decrease.

### Example 7: Epitope Mapping and Fine-Mapping

Epitope mapping and fine-mapping were performed essentially as described (Timmerman et al., Functional reconstruction and synthetic mimicry of a conformational epitope using CLIPS™ technology. J. Mol. Recognit. 20 (2007) 283-99; Slootstra et al., Structural aspects of antibody antigen interaction revealed through small random peptide libraries, Molecular Diversity 1: 87 (1996) 96). In brief, CLIPS technology structurally fixes peptides into defined three-dimensional structures. This results in functional mimics of even the most complex binding sites. CLIPS technology is now routinely used to shape peptide libraries into single, double or triple looped structures as well as sheet and helix-like folds.

CLIPS library screening starts with the conversion of the target protein into a library of up to 10,000 overlapping peptide constructs, using a combinatorial matrix design. On a solid carrier, a matrix of linear peptides is synthesized, which are subsequently shaped into spatially defined CLIPS constructs. Constructs representing both parts of the discontinuous epitope in the correct conformation bind the antibody with high affinity, which is detected and quantified. Constructs presenting the incomplete epitope bind the antibody with lower affinity, whereas constructs not containing the epitope do not bind at all. Affinity information is used in iterative screens to define the sequence and conformation of epitopes in detail.

The following clones were analyzed: clone-02, clone-03, clone-04, clone-06, and clone-10. The following target sequences of CD3 (N-terminal sequences) were used

| HumanCD3 (SEQ ID NO: 46): | | |
|---|---|---|
| 2 | DGNEEMGGIT QTPYKVSISG TTVILTCPQY PGSEILWQHN DKNIGGDEDD | 51 |
| 52 | KNIGSDEDHL SLKEFSELEQ SGYYVCYPRG SKPEDANFYL YLRARVCENC | 101 |
| 102 | MEMD | 105 |
| Cynomolgus CD3 (SEQ ID NO: 47): | | |
| 2 | DGNEEMGSIT QTPYQVSISG TTVILTCSQH LGSEAQWQHN GKNKEDSGDR | 51 |
| 52 | LFLPEFSEME QSGYYVCYPR GSNPEDASHH LYLKARVCEN CMEMD | 96 |

| Sequence alignments | |
|---|---|
| CLUSTAL 2.1 multiple sequence alignment: | |
| Human | DGNEEMGGITQTPYKVSISGTTVILTCPQYPGSEILWQHNDKNIGGDEDDKNIG |
| | \|\|\|\|\|\|\|.\|\|\|\|\|\|:\|\|\|\|\|\|\|\|\|\|\|\|.\|: \|\|\| \|\|\|\|.\|\| \|\|. \| |
| Cynomolgus | DGNEEMGSITQTPYQVSISGTTVILTCSQHLGSEAQWQHNGKNK---EDS---G |
| Human | SDEDHLSLKEFSELEQSGYYVCYPRGSKPEDANFYLYLRARVCENCMEMD |
| | \|:\| \| \|\|\|\|:\|\|\|\|\|\|\|\|\|\|\|\|\|:\|\|\|\| :\|\|\|:\|\|\|\|\|\|\|\|\|\|\| |
| Cynomolgus | ---DRLFLPEFSEMEQSGYYVCYPRGSNPEDASHHLYLKARVCENCMEMD |

### Synthesis of Peptides

To reconstruct discontinuous epitopes of the target molecule a library of structured peptides was synthesized. This was done using the so-called “Chemically Linked Peptides on Scaffolds” (CLIPS) technology. CLIPS technology allows structuring peptides into single loops, double loops, triple loops, sheet like folds, helix like folds and combinations thereof. CLIPS templates are coupled to cysteine residues. The side chains of multiple cysteines in the peptides are coupled to one or two CLIPS templates. For example, a 0.5 mM solution of the T2 CLIPS template 1,3 bis (bromomethyl) benzene is dissolved in ammonium bicarbonate (20 mM, pH 7.9)/acetonitrile (1:1(v/v). This solution is added onto the peptide arrays. The CLIPS template will bind to side chains of two cysteines as present in the solid phase bound peptides of the peptide arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt buffer containing 1 percent SDS/0.1 percent beta mercaptoethanol in PBS (pH 7.2) at 70° C. for 30 min, followed by sonication in H₂O for another 45 min. The T3 CLIPS carrying peptides were made in a similar way but now with three cysteines.

### ELISA Screening

The binding of antibody to each of arrays were incubated with primary antibody solution (overnight at 4° C.). After washing, the peptide arrays were incubated with a 1/1000 dilution of an antibody peroxidase conjugate (SBA, cat.nr.2010 05) for one hour at 25° C. After washing, the peroxidase substrate 2,2′ azino di 3 ethylbenzthiazoline sulfonate (ABTS) and 2 μl/ml of 3% H₂O₂ were added. After one hour, the color development was measured. The color development was quantified with a charge coupled device (CCD) camera and an image processing system.

### Design of Peptides

Chemically synthesized CLIPS peptides were synthesized as described above according to the following designs.

### Set 1

### Mimic Type Linear Peptides

### Description:

Double sets of linear peptides for both human and cynomolgus sequences. Length is 15 residues with an overlap of 14. Two of the sets feature a double alanine mutation (shown in grey).

Sequences (first 10 of human sequences shown)

```
DGNEEMGGITQTPYK (SEQ ID NO: 48)
  GNEEMGGITQTPYKV (SEQ ID NO: 49)
    NEEMGGITQTPYKVS (SEQ ID NO: 50)
      EEMGGITQTPYKVSI (SEQ ID NO: 51)
        EMGGITQTPYKVSIS (SEQ ID NO: 52)
          MGGITQTPYKVSISG (SEQ ID NO: 53)
            GGITQTPYKVSISGT (SEQ ID NO: 54)
              GITQTPYKVSISGTT (SEQ ID NO: 55)
                ITQTPYKVSISGTTV (SEQ ID NO: 56)
                  TQTPYKVSISGTTVI (SEQ ID NO: 57)

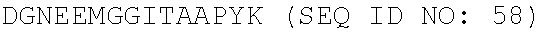

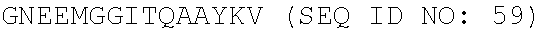

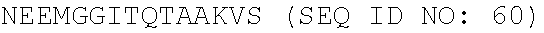

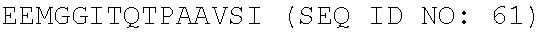

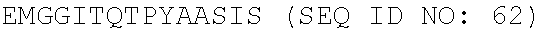

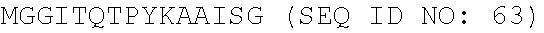

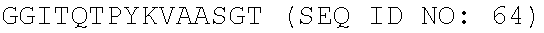

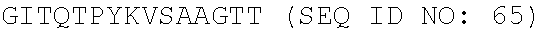

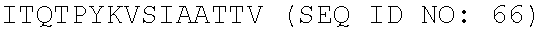

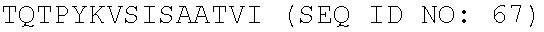










```

### Set 2

Mimic Type Linear Peptides with Added Charges

### Description:

Control sets with added charges that are required for some antibodies that strongly interact with the peptide array surface

Sequences (first 10 of human sequence shown)

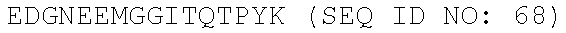

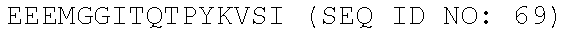

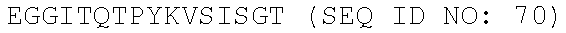

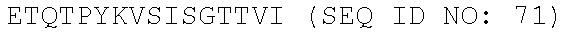

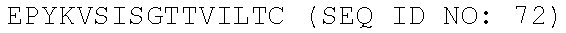

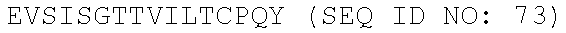

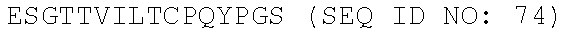

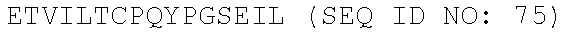

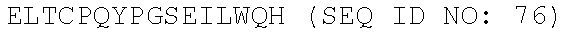

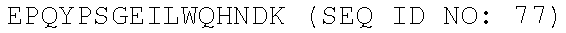

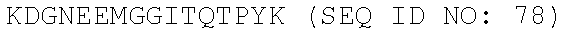

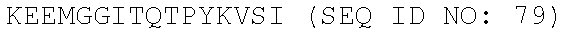

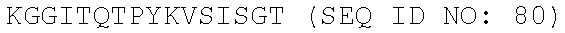

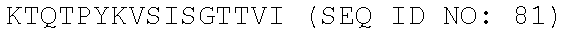

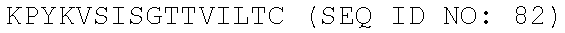

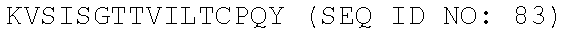

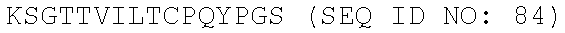

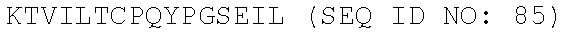

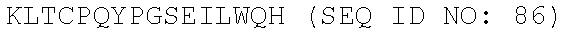

### Set 3

### Mimic Type Conformational Peptides

### Description:

Peptide sequence are similar to Set 1, but are constrained into a CLIPS conformational loop.

Sequences
(first 10 of unmodified human sequence shown)

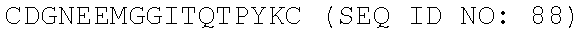

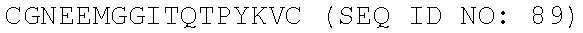

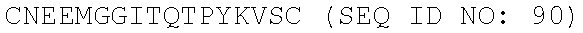

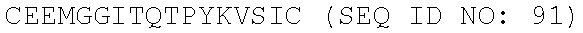

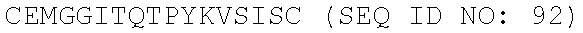

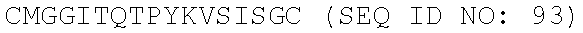

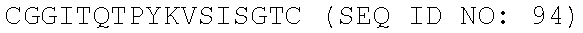

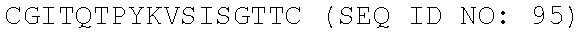

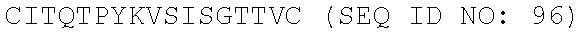

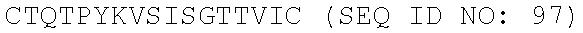

### Set 4

### Mimic Type: CLIPS Conformational Peptides

### Description:

Overlapping set of 20mer CLIPS conformational peptides

Sequences (first 10 of human sequence shown)

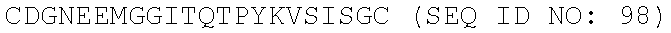

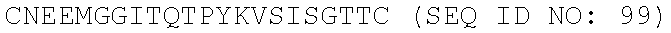

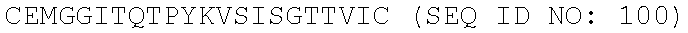

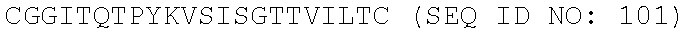

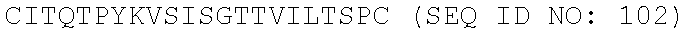

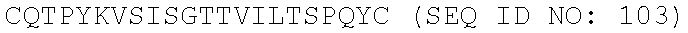

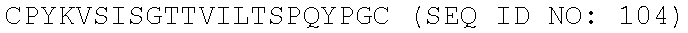

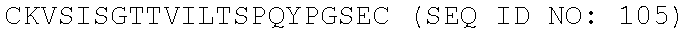

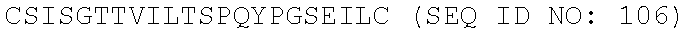

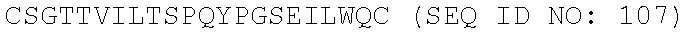

### Set 5

### Mimic Type:CLIPS Discontinuous Matrix Peptides

### Description:

Combinatorial set of 13mer peptides, constrained pairwise into a double looped CLIPS structure. Human and Cynomolgus peptides are ordered according to pairwise alignment to minimize technical variation.

Sequences (first 10 shown)

| | |
|---|---|
| 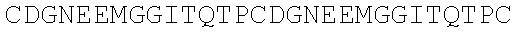 | (SEQ ID NO: 108) |
| 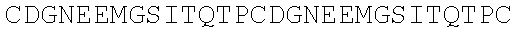 | (SEQ ID NO: 109) |
| 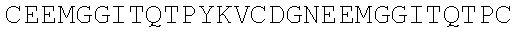 | (SEQ ID NO: 110) |
| 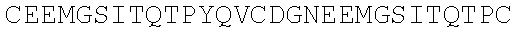 | (SEQ ID NO: 111) |
| 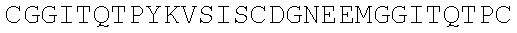 | (SEQ ID NO: 112) |
| 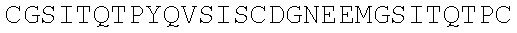 | (SEQ ID NO: 113) |
| 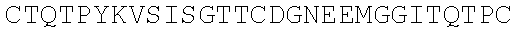 | (SEQ ID NO: 114) |
| 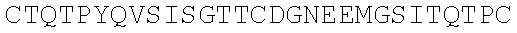 | (SEQ ID NO: 115) |
| 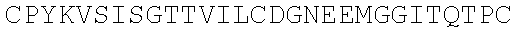 | (SEQ ID NO: 116) |
| 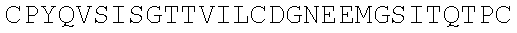 | (SEQ ID NO: 117) |

### Identification of Putative Epitopes

In general, all five antibodies showed very similar binding characteristics. All binding took place on the N terminus of human CDD3ε (data not shown). Considering the binding strength and observations from constrained and non-constrained peptides, it is most likely that all antibodies bind predominantly to linear epitopes as:
Binding was observed only to N-terminal sequences
Loss of D2 or G3 does not strongly reduce binding
Loss of 2DGN4 completely abolishes binding.

### CONCLUSION

The analysis identified binding regions for all five antibodies tested. All antibodies were found to bind to a seemingly linear epitope on the N terminus. All antibodies were found to bind to a similar epitope that relied strongly on 2DGN4 for binding.

### Example 8: Epitope Fine-Mapping

### Methods

15mer linear arrays derived from human and cynomolgus CD3ε, residues 2-16 and 5-20, in which each position is substituted by 18 amino acids (all natural amino acids except cysteine) were probed with the antibodies and specificities affecting the binding were found.

### Results

All antibodies bind the N terminus with an absolute requirement for N4 and an involvement of E6, and share significant similarities. All antibodies bind both human and cynomolgus versions of CD3ε, despite the small differences in sequence adjacent to the core epitope.

### Target Protein

The initial mapping identified a linear stretch on the N terminus of

CD3ε as the core epitope for all antibodies tested. Residues 2-20 of the sequences below were used to design full substitution libraries of linear 15mer peptides.

### Methods

### Synthesis of Peptides

Linear peptides were synthesized by standard Fmoc synthesis on to the hydrogel of a Hi-Sense surface. After deprotection and washing, the cards were extensively washed in a sonication bath with a proprietary washing buffer.

### ELISA Screening

The binding of the antibodies to each of the synthesized peptides was tested by ELISA. The peptide arrays were incubated with primary antibody solution (overnight at 4° C.). After washing, the peptide arrays were incubated with a 1/1000 dilution of an antibody peroxidase conjugate (SBA, cat.nr.2010-05) for one hour at 25° C. After washing, the peroxidase substrate 2,2′-azino-di-3-ethylbenzthiazoline sulfonate (ABTS) and 2 μl/ml of 3% H₂O₂ were added. After one hour, the color development was measured. The color development was quantified with a charge coupled device (CCD)—camera and an image processing system.

### Design of Peptides

Chemically synthesized CLIPS peptides were synthesized (see also Methods section) according to the following designs.

### Set 1

### Mimic Type: Linear Peptides

### Description:

Linear 15mer peptides derived from human CD3ε residues 2-16. In each peptide one of the residues is replaced by all naturally occurring amino acids (except cysteine), creating a saturation mutagenesis library.

Sequences (first 10 shown)

(SEQ ID NO: 118)

AGNEEMGGITQTPYK

(SEQ ID NO: 119)

DGNEEMGGITQTPYK

(SEQ ID NO: 120)

GGNEEMGGITQTPYK

(SEQ ID NO: 121)

HGNEEMGGITQTPYK

(SEQ ID NO: 122)

LGNEEMGGITQTPYK

(SEQ ID NO: 123)

MGNEEMGGITQTPYK

(SEQ ID NO: 124)

NGNEEMGGITQTPYK

(SEQ ID NO: 125)

PGNEEMGGITQTPYK

(SEQ ID NO: 126)

QGNEEMGGITQTPYK

(SEQ ID NO: 127)

RGNEEMGGITQTPYK

### Set 2

### Mimic Type: Linear Peptides

### Description:

Linear 15mer peptides derived from cynomolgus CD3ε residues 2-16. In each peptide one of the residues is replaced by all naturally occurring amino acids (except cysteine), creating a saturation mutagenesis library.

Sequences (first 10 shown)

(SEQ ID NO: 128)

AGNEEMGSITQTPYQ

(SEQ ID NO: 129)

DGNEEMGSITQTPYQ

(SEQ ID NO: 130)

GGNEEMGSITQTPYQ

(SEQ ID NO: 131)

HGNEEMGSITQTPYQ

(SEQ ID NO: 132)

LGNEEMGSITQTPYQ

(SEQ ID NO: 133)

MGNEEMGSITQTPYQ

(SEQ ID NO: 134)

NGNEEMGSITQTPYQ

(SEQ ID NO: 135)

PGNEEMGSITQTPYQ

(SEQ ID NO: 136)

QGNEEMGSITQTPYQ

(SEQ ID NO: 137)

RGNEEMGSITQTPYQ

### Set 3

### Mimic Type: Linear Peptides

### Description:

Linear 15mer peptides derived from human CD3ε residues 5-20. In each peptide one of the residues is replaced by all naturally occurring amino acids (except cysteine), creating a saturation mutagenesis library.

Sequences (first 10 shown)

| | |
|---|---|
| 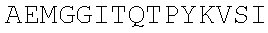 | (SEQ ID NO: 138) |
| 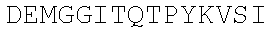 | (SEQ ID NO: 139) |
| 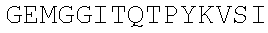 | (SEQ ID NO: 140) |
| 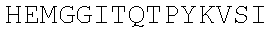 | (SEQ ID NO: 141) |
| 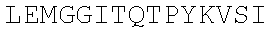 | (SEQ ID NO: 142) |
| 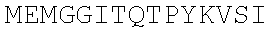 | (SEQ ID NO: 143) |
| 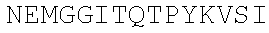 | (SEQ ID NO: 144) |
| 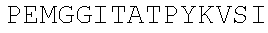 | (SEQ ID NO: 145) |
| 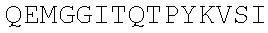 | (SEQ ID NO: 146) |
| 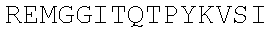 | (SEQ ID NO: 147) |

### Set 4

### Mimic Type: Linear Peptides

### Description;

Linear 15mer peptides derived from cynomolgus CD3ε residues 5-20. In each peptide one of the residues is replaced by all naturally occurring amino acids (except cysteine), creating a saturation mutagenesis library.

Sequences (first 10 shown)

(SEQ ID NO: 148)

AEMGSITQTPYQVSI

(SEQ ID NO: 149)

DEMGSITQTPYQVSI

(SEQ ID NO: 150)

GEMGSITQTPYQVSI

(SEQ ID NO: 151)

HEMGSITQTPYQVSI

(SEQ ID NO: 152)

LEMGSITQTPYQVSI

(SEQ ID NO: 153)

MEMGSITQTPYQVSI

(SEQ ID NO: 154)

NEMGSITQTPYQVSI

(SEQ ID NO: 155)

PEMGSITQTPYQVSI

(SEQ ID NO: 156)

QEMGSITQTPYQVSI

(SEQ ID NO: 157)

REMGSITQTPYQVSI

### Comparison of Samples

All five antibodies bind to linear peptides derived from the human and cynomolgus variant of the CD3ε N terminus in a very similar fashion, by absolutely requiring N4 (only to be supplanted by Histidine), and with a great preference for E6, for which limited substitutions are tolerated, however it seems that Glutamate is the most preferred residue at that position. None of the antibodies bound to peptides spanning residues 5-20. Within this group of five, three antibodies (Clone 2, Clone 3, and Clone 4) are more sensitive to mutations in the Cyno sequence than the other two (Clone 6, and Clone 10), in that the former group of three also is more sensitive to replacements of G3, E5, and/or G8. This observation is in line with the difference in affinity for the human and cynomolgus forms of the protein as determined by SPR (see Table 1).

### Conclusion

The analysis fine mapped the epitopes of the five antibodies, which bind the N terminus with an absolute requirement for N4 and E6, and share significant similarities. All antibodies bind both human and cynomolgus versions of CD3ε, despite the small differences in sequence adjacent to the core epitope.

### Example 9: Cytokine Release

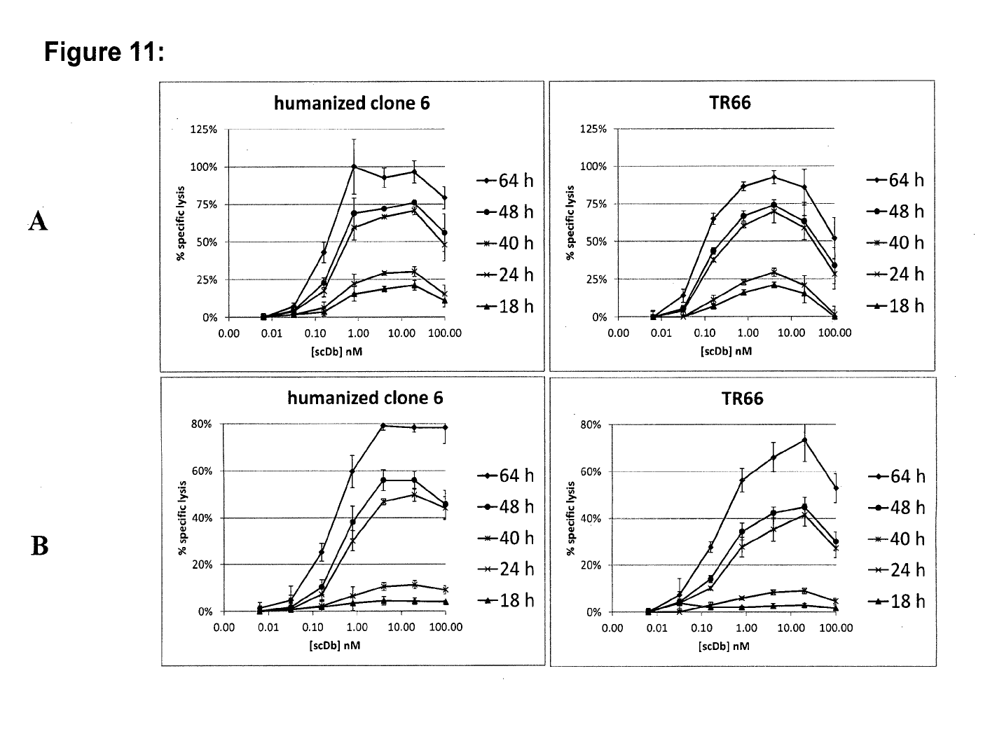

Human CD8+ T cells freshly isolated from buffy coats were incubated with CHO cells expressing human interleukin-5 receptor (hIL-5R) in an effector-to-target ratio of 10:1, with increasing concentrations of bispecific anti-IL5R×CD3ε single-chain diabodies (scDb). Both scDbs tested contained identical IL5R binding domains (VL: SEQ ID NO: 29; VH: SEQ ID NO: 30), but different CD3ε binding domains. The CD3ε binding domains tested were the variable domains of Numab's humanized clone 6 (VL: SEQ ID NO: 21; VH: SEQ ID NO: 22) and TR66 (Moore et al, Blood. 2011; 117:4542-4551). Specific lysis of target cells was assessed at various time points as described in the methods section. As depicted in FIG. 11, both scDbs showed a similar EC₅₀ in the dose-response curve at 64 hours, with 0.31 nM and 0.19 nM for the scDb containing Numab's anti-CD3 domain and TR66, respectively. However, the scDb containing Numab's anti-CD3 domain led to higher maximal lysis of target cells and reached maximal specific lysis at lower concentrations. Furthermore, there was a clear reduction of target cell lysis at high concentrations with the scDb containing TR66, which was essentially absent with Numab's anti-CD3 domain. The narrow bell-shaped dose-response curve for the scDb containing the variable domain of TR66 suggests that a tightly controlled dosing scheme is required for therapeutic applications to keep systemic levels in the active concentration window. In contrast, Numab's anti-CD3 domain would potentially lead to a broader therapeutic window.

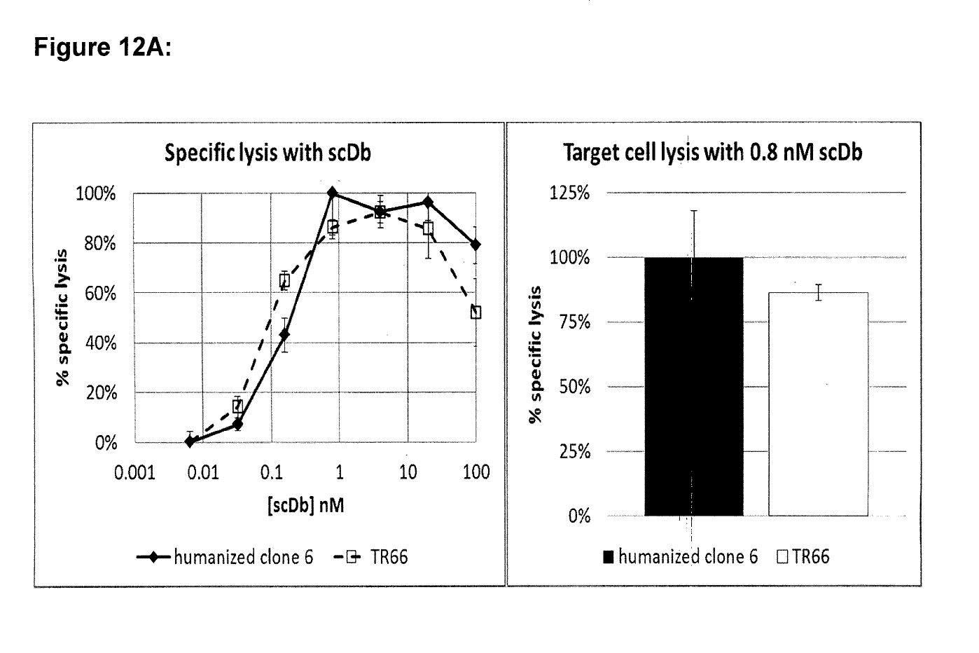

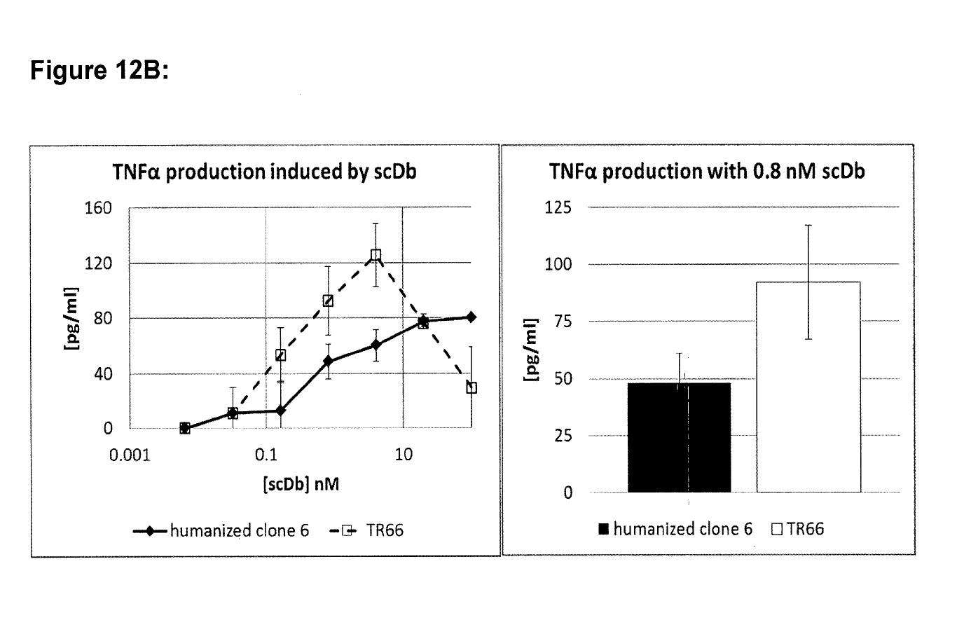

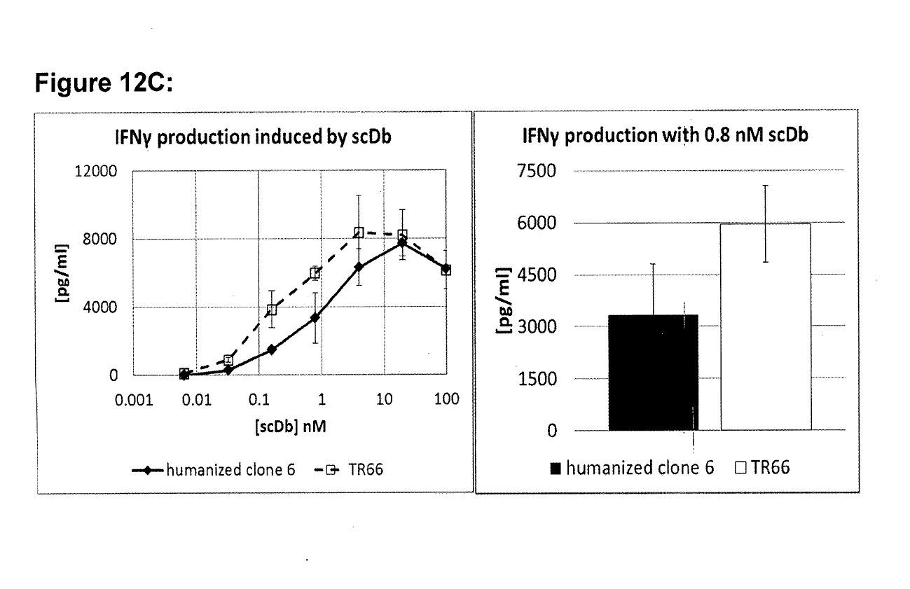

Cytokine concentrations were measured from the culture supernatants of the experiment described above. Although, showing similar potency to induce lysis of target cells, the two scDbs profoundly differed in their effects on cytokine production. While the scDb containing the variable domain of TR66 led to a steep dose-dependent increase of TNFα, there was a much reduced TNFα production observed with the scDb containing Numab's anti-CD3 domain at all effective concentrations. At 0.8 nM scDb, the lowest concentration at which the scDb containing Numab's anti-CD3 domain reached maximal target cell lysis, TNFα concentrations reached only about 50% of the concentrations produced with the scDb containing the variable domain of TR66 (FIG. 12B). Further, in correlation to the reduced lytic activity at high concentrations shown in FIG. 12A, TNFα production dropped at the highest concentration only with the scDb containing the variable domain of TR66. In line with these observations, also IFNγ production was significantly lower with the scDb containing Numab's anti-CD3 domain, with about 50% of the levels reached with the scDb containing the variable domain of TR66 at a concentration of 0.8 nM (FIG. 12C).

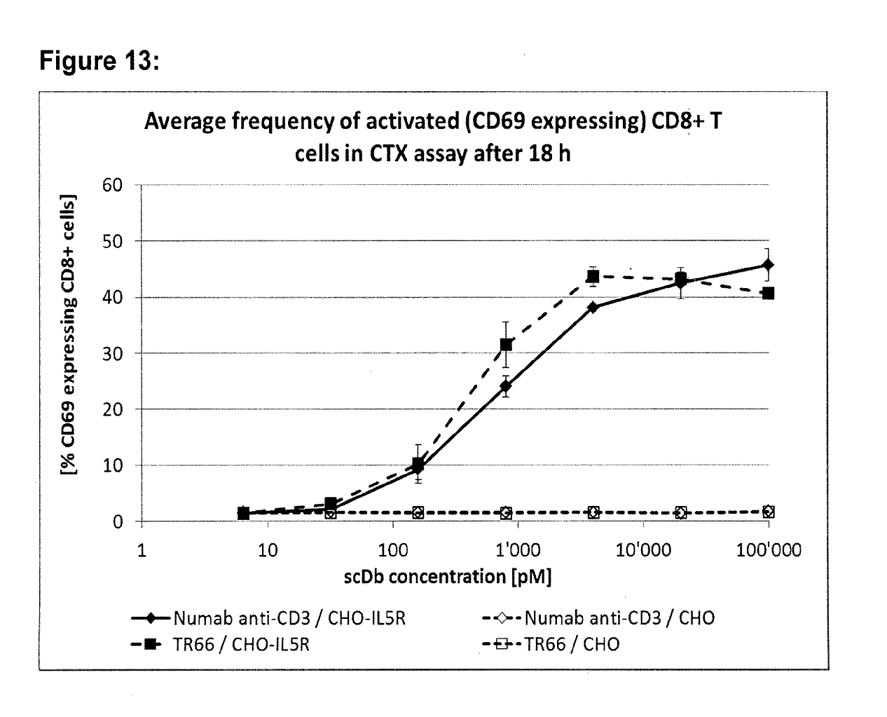

In order to explain the apparent loss of lytic potential of CD8+ T cells in presence of high concentrations of the scDb containing the variable domain of TR66 (see FIGS. 11 and 12A), we characterized the potential of both scDbs to activate CD8+ T cells by determining the percentage of T cells expressing CD69, an early marker for T cell activation. Both scDbs induced CD69 expression on CD8+ T cells in a dose dependent manner at concentrations up to 4 nM, with the scDb containing Numab's anti-CD3 domain leading to slightly lower percentage of activated CD8+ cells at low concentrations. In line with the observed loss in target cell lysis, the percentage of activated T cells decreased at high concentrations for the scDb containing the variable domain of TR66. In contrast, with the scDb containing Numab's anti-CD3 domain, the percentage of CD69 expressing T cells continued to increase at all tested concentrations (up to 100 nM) (FIG. 13). This together with the steadily increasing TNFα concentrations (FIG. 12B) suggests that total T cell activity is still increasing in a dose-dependent manner over the entire range of tested concentrations of the scDb containing Numab's anti-CD3 domain, whereas the maximum T cell activity with the scDb containing the variable domain of TR66 is reached at a concentration of about 4 nM.

### Example 10: Active Induction of Mouse Experimental Autoimmune Encephalomyelitis in SJL Mice

### Experiment:

Experimental autoimmune encephalomyelitis (EAE) has been actively induced by peptide immunization as described elsewhere (Miller et al., Curr Protoc Immunol. 2007; Chapter:Unit-15.1.doi:10.1002/0471142735.iml1501s77). Clinical symptoms were assessed on a daily basis using EAE symptom scoring ranging from 0 (no detectable signs of EAE) to 5 (dead). As indicated below, the two bispecific anti-IL23R×CD3 antibody constructs PRO386 and PRO387 were tested in a prophylactic as well a therapeutic setting. Prevention groups were dosed by daily intraperitoneal injection of PRO386 and PRO387 from day 1 after induction until day 8. The therapy groups received daily intraperitoneal injections of 50 mcg of PRO386, PRO387 and an anti-CD3 Fab as a control, for 11 days after onset of the disease (score of ˜0.5).

| | | Group | | | |
|---|---|---|---|---|---|
| | | size | Test | Treatment | Dose |
| Group | Strain | (n) | substance | design | (mcg) |
| a | SJL | 5 | PBS | prevention/ | 0 |
| | | | | prophylaxis | |
| b | SJL | 5 | PRO386 | prevention/ | 50 |
| | | | (Tribody) | prophylaxis | |
| d | SJL | 5 | PRO387 (scDb) | prevention/ | 50 |
| | | | | prophylaxis | |
| c | SJL | 5 | PRO386 | therapy | 50 |
| | | | (Tribody) | | |
| e | SJL | 5 | PR0387 (scDb) | therapy | 50 |
| f | SJL | 3 | anti-CD3 Fab | therapy | 50 |

### Results:

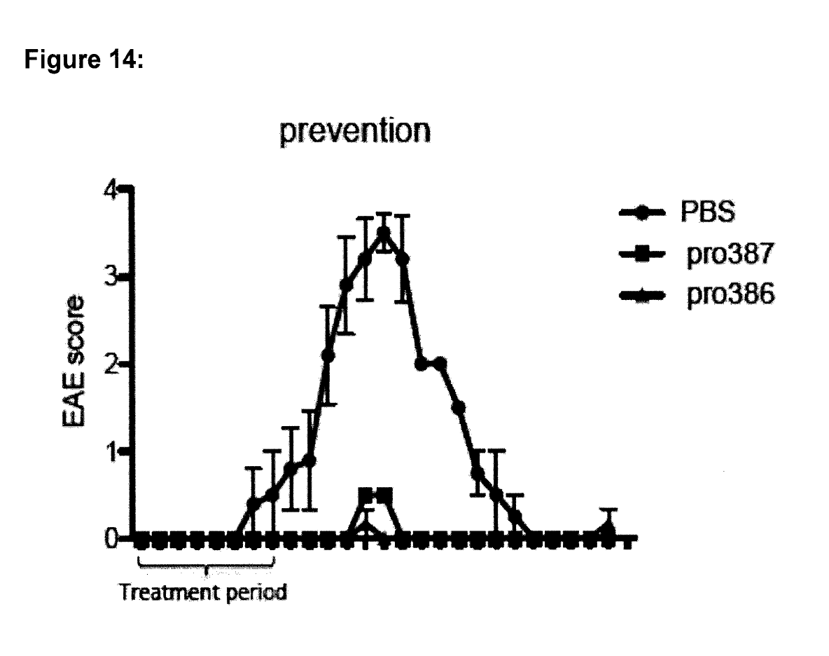

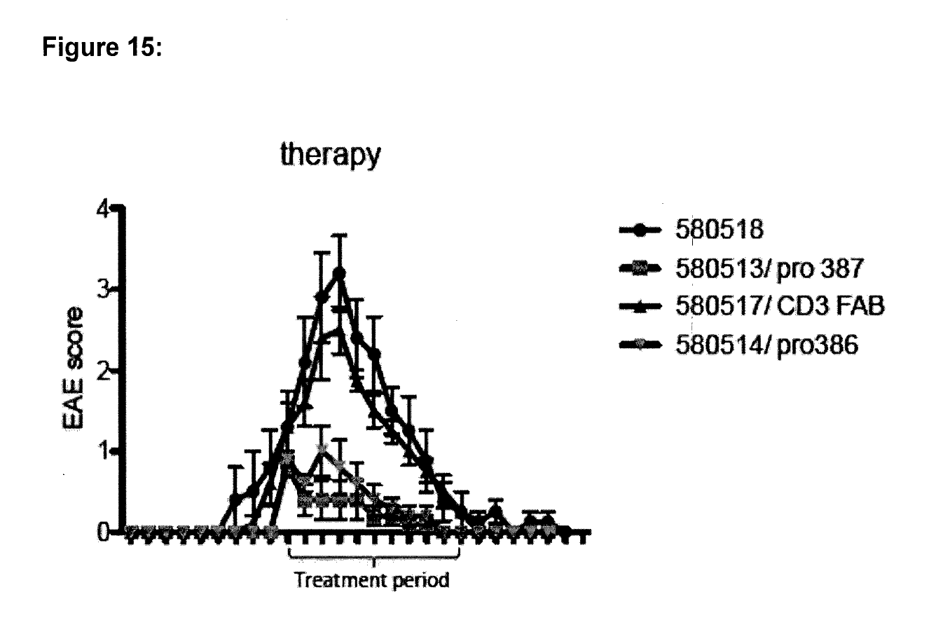

Disease symptoms, as assessed by EAE scoring peaked at around day 14 for the PBS control group. Both bispecific constructs PRO386 and PRO387 blocked the development of clinical symptoms nearly completely when administered in a prophylactic setting before disease onset (FIG. 14). This is in line with the findings by Chen et al., J Clin Invest. 2009; 116:1317-1325) that antibodies blocking IL-23 alone (anti-IL-23 p19) or IL-12 and IL-23 together (anti-IL-12 p40) were able to block disease symptoms in a prophylactic setting. Surprisingly, and in contrast to anti-IL-12 p40 or anti-IL-23 p19 antibodies, both, PRO386 and PRO387 were able to ameliorate disease symptoms when administered in a therapeutic setting, starting after onset of the disease (FIG. 15). A probable explanation for this is that IL-23 blockade inhibits the de novo differentiation of pathogenic T cells, such as IL23R expressing Th17 and gamma-delta T cells. In established disease conditions, however, these cells are terminally differentiated and my no longer depend on IL-23 signaling. Thus, IL-23 blockade cannot block activity of all disease driving cell types. In contrast, depletion of IL23R expressing cells eliminates the key disease driving cells independent of their level of differentiation.

### Example 11: Engineering and Characterization of a Bispecific Single-Chain Diabody Surrogate (scDb)

With the aim to redirect murine CD3ε+ T cells to lyse IL23R expressing target cells, a bispecific antibody fragment of the single-chain diabody (scDb) format was engineered. This construct termed PRO387 contains the VH and VL of the anti-IL23R scFv 14-11-D07-sc01, as well as the VH and VL of a hamster anti-CD3ε antibody (2C11). The mouse reactive 2C11 has been described in the literature (Leo 0, et al. 1987. P. Natl. Acad. Sci. USA 84:1374) and has been extensively characterized so that structural and sequence information is available (Fernandes R A, et al. 2012. J. Biol. Chem. 287: 13324-13335).

Affinities of the anti-IL23R and anti-CD3ε binding moieties towards mouse IL23R, and mouse CD3ε, respectively were measured by SPR (Table 1).

TABLE 1

| | mouse IL23R | | | mouse CD3□ | | |
|---|---|---|---|---|---|---|
| | k_{d} | K_{D} | K_{D} | kₐ | k_{d} | K_{D} |
| scDb ID | [s⁻¹] | [M] | [M] | [M⁻¹ s⁻¹] | [s⁻¹] | [M] |
| PRO387 | 7.01E+05 | 3.26E−03 | 4.66E−09 | 2.33E+05 | 2.13E−03 | 9.11E−09 |

### Example 12: Engineering and Characterization of a Bispecific, Trivalent Fab-scFv Fusion Surrogate Molecule and Anti-mCD3 Fab

With the aim to redirect murine CD3ε+ T cells to lyse IL23R expressing target cells, a bispecific antibody fragment, a Fab-scFv fusion (EP1049787 B1) format, was engineered. This heterodimeric construct termed PRO386 contains the Fab fragment of the mouse reactive 2C11 (VL-CL & VH-CH1), both chains are C-terminally fused to an anti-IL23R scFv 14-11-D07-sc01 via a flexible linker.

In addition to the bispecific molecule a mouse reactive anti-CD3 Fab (PRO400) was constructed devoid of the fused scFv modules.

Affinities of the anti-IL23R and anti-CD3ε binding moieties towards mouse IL23R, and mouse CD3ε, respectively were measured by SPR (Table 2).

TABLE 2

| | mouse IL23R | | | mouse CD3□ | | |
|---|---|---|---|---|---|---|
| | k_{d} | K_{D} | K_{D} | kₐ | k_{d} | K_{D} |
| scDb ID | [s⁻¹] | [M] | [M] | [M⁻¹ s⁻¹] | [s⁻¹] | [M] |
| PRO386 | 9.15E+05 | 5.33E−04 | 5.83E−10 | 1.71E+05 | 1.96E−03 | 1.15E−08 |
| PRO400 | — | — | — | 2.33E+05 | 2.66E−03 | 1.14E−08 |

### Example 13: Treatment of SJL Mice with Actively Induced Experimental Autoimmune Encephalomyelitis

### Treatment Schedule:

### End of Experiment: Day 25 Post Cell Transfer.

| Host | Group | Donor | | | | |
|---|---|---|---|---|---|---|
| mice | size | mice | Molecule | Therapy | Dose | Treatment schedule |
| C57B/6 | 3 | C57B/6 | PRO400 | Therapy | 50 μg | Daily i.p. injection for eight |
| | | | | | | consecutive days starting |
| | | | | | | when animals reached a score |
| | | | | | | of 1 |
| C57B/6 | 4 | C57B/6 | αp40 | Prevention | 200 μg | Daily i.p. injection starting |
| | | | mAb | | ip | from day of cell transfer until |
| | | | | | | day 8 |
| C57B/6 | 3 | C57B/6 | PRO386 | Therapy | 50 μg | Daily i.p. injection starting |
| | | | | | ip | from day of cell transfer until |
| | | | | | | day 8 |
| C57B/6 | 1 | C57B/6 | αp40 | Therapy | 200 μg | Daily i.p. injection for eight |
| | | | mAb | | ip | consecutive days starting |
| | | | | | | when animals reached a score |
| | | | | | | of 1 |

### Readout:

Progression of neuropathologic symptoms
Cytokine production by brain infiltrating CD4 T cells
Cell numbers of brain infiltrating CD4 and CD8 T cells

The development of clinical symptoms in C57B/6 mice treated with CD3FAB, PRO386 and anti-p40 antibody according to schedule (n=one-four animals per group) after adoptive transfer of lymphocytes from previously MOG/CFA immunized C57B/6 animals was monitored. Each dot represents one mouse. Results represent mean+/−SEM. For analysis of EAE score plots comparing PBS treated group against the PRO386 treated group, 2 way ANOVA with Bonferroni's post-test was used.

Individual Raw Data: Clinical EAE Scores

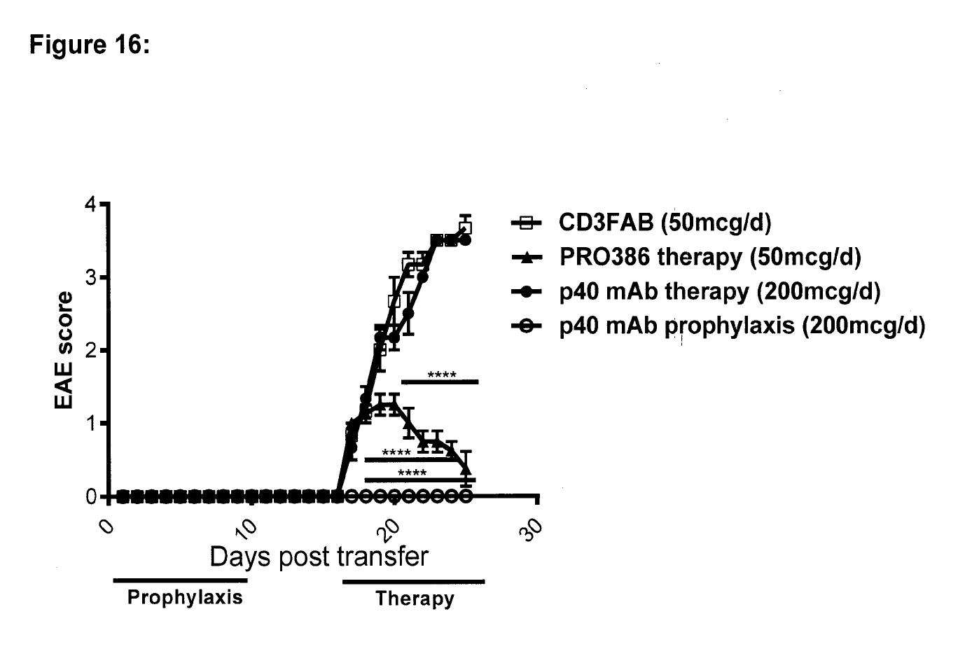

The timeline of clinical symptom progression is shown in FIG. 16. Mice treated with 50 μg/d CD3FAB or 200 μg/d anti-p40 antibody starting after disease onset (clinical score of one) on day 17 displayed progressing paralysis. However, injection of 50 μg/d PRO386 after the disease onset (clinical score of one) prevented disease progression and even ameliorated disease symptoms. Mice treated with 200 μg/d anti-p40 antibody just after the cell transfer showed no disease symptoms. This validates the results from study 1 and suggests that disease progression becomes independent of IL23 signaling after manifestation of clinical symptoms.

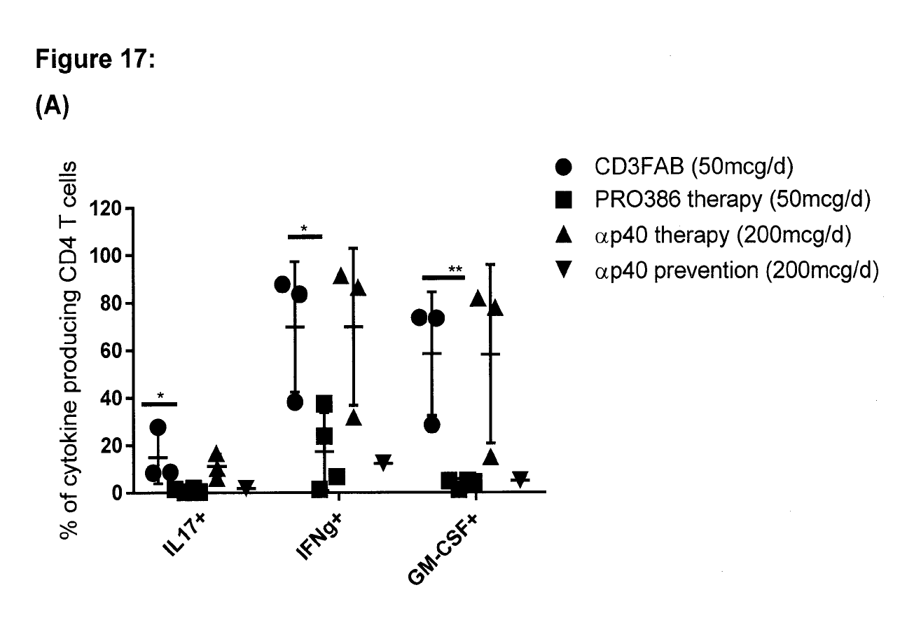

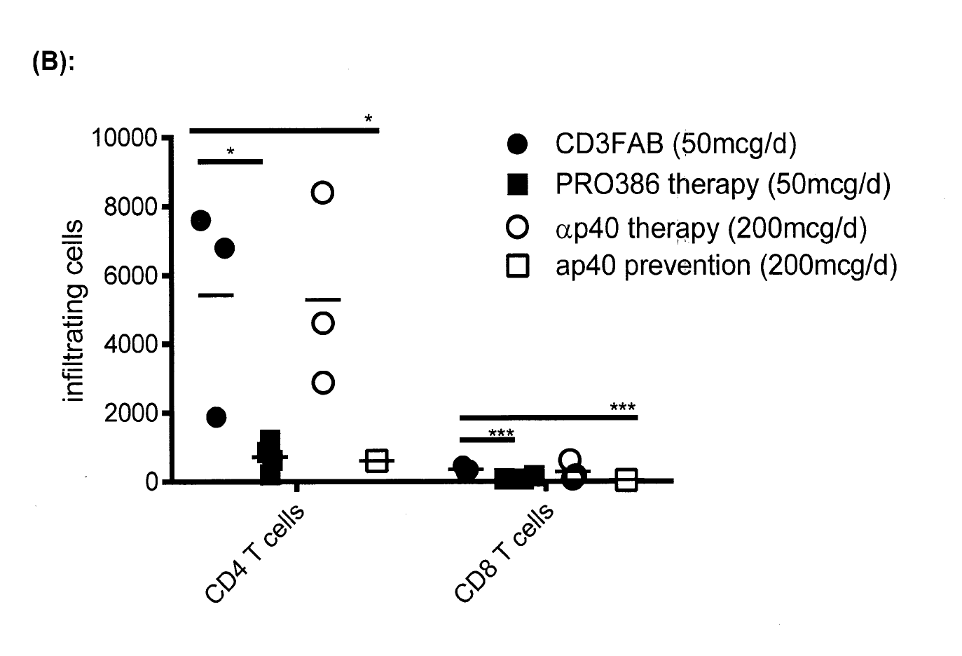

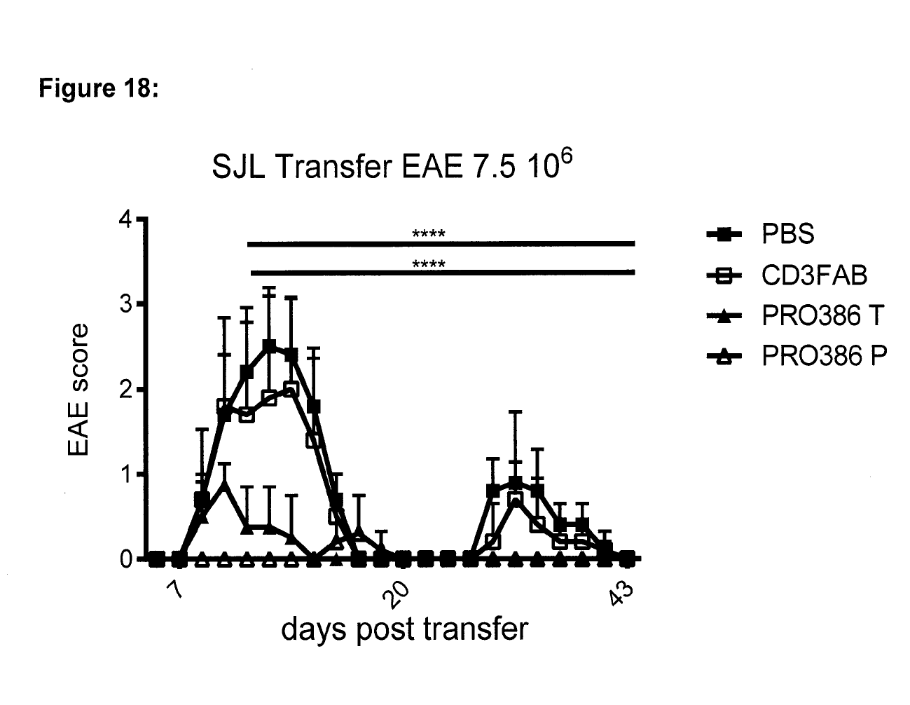

The number of cytokine expressing T cells in the brain of animals at the peak of the disease (day 20) in percents is shown in FIG. 17(A) and in absolute numbers of infiltrating T cells in FIG. 17(B): Animals therapeutically treated with PRO386 after the onset of clinical disease symptoms displayed reduced infiltration of cytokine producing CD4 T cells in percents and absolute numbers. IFNγ and GM-CSF producing CD4+ T cells were strongly reduced in the brain of animals treated with PRO386. This finding correlates with the clinical score in the respective group. Only prophylactic anti-p40 antibody treatment was sufficient to reduce the percentage and number of cytokine expressing T cells in the brain. This supports the hypothesis that IL23 is important for early differentiation of Type-17 T cells but has no relevant function after terminal differentiation of these cells and the manifestation of clinical symptoms.

### Example 13: Efficacy of PRO386 Monotherapy to Prevent EAE Relapses in a SJL Model for RR-EAE

The aim of the study was to test the efficacy of PRO386 monotherapy in a setting of passive relapse remitting transfer EAE to prevent disease onset and to ameliorate disease symptoms after clinical disease manifestation. PRO386 is a bispecific anti-IL23R×CD3 Tribody (Tb) in which two anti-IL23R scFvs are added C-terminally to an anti-CD3 Fab. As controls either PBS or a monospecific anti-CD3 Fab fragment were used.

### Treatment Schedule:

Injection of 7.5×10⁶ cells (to prevent fatal course of disease in control group); end of experiment: after second flare post cell transfer.

| Host | Group | Donor | | | | |
|---|---|---|---|---|---|---|
| mice | size | mice | Molecule | Therapy | Dose | Treatment schedule |
| SJL | 5 | SJL | PBS | Prevention | 200 μl | Daily i.p. injection starting from |
| | | | | | PBS | day of cell transfer until day 16 |
| SJL | 5 | SJL | CD3FAB | Prevention | 50 μg | Daily i.p. injection starting from |
| | | | | | | day of cell transfer until day 8 |
| SJL | 5 | SJL | PRO386 | Prevention | 50 μg | Daily i.p. injection starting from |
| | | | | | | day of cell transfer until day 8 |
| SJL | 5 | SJL | PRO386 | Therapy | 50 μg | Daily i.p. injection starting from |
| | | | | | | day 9 transfer until day 17 |

### Readout:

### Progression of Neuropathologic Symptoms

### Results:

Mice treated with PBS or CD3FAB starting at the day of cell transfer displayed first clinical symptoms around day 8. In both groups the EAE score peaked around day 11 and spontaneously declined to 0 at day 18. These animals experienced the second relapse starting around day 27 and peaking at day 31.

Mice prophylactically treated with 50 μg of PRO386 (prevention) had a delay in the disease onset (starting around day 17) and experienced very mild symptoms in comparison to the previous groups. These mice were protected from a relapse.

A forth group of animals received 50 μg/d PRO386 as a therapy after the onset of clinical disease, starting from day 9 until day 19. These animals displayed amelioration of disease symptoms and complete protection from a relapse.

### Conclusion:

PRO386 is highly effective in inhibiting the disease onset of passive EAE as well as ameliorating clinical symptoms after the onset of disease manifestation. Both, prophylactic and therapeutic intervention with PRO386 protected from secondary relapses.

### General Methods:

### Primary Sequence Analysis

The obtained sequence information of the corresponding heavy and light chain variable domains (VL and VH) was aligned and grouped according to sequence homology. The sets of rabbit variable domains were analyzed to identify unique clones and unique sets of CDRs. A combined alignment of the VL and VH domains was performed based on the joint amino acid sequences of both domains to identify unique clones. In addition to the alignment of the variable domains, the set of sequences of the six complementarity determining regions (CDRs) of each rabbit IgG clone were compared between different clones to identify unique sets of CDRs. These unique CDR sets were aligned using the multiple alignment tool COBALT and a phylogenetic tree was generated with the Neighbor Joining algorithm. The CDR sets were grouped based on sequence homology of the joined CDR sequences of each clone and a cluster threshold was determined based on sequence homology and identity. Based on the screening assay results and the cluster affiliation of the individual rabbit IgG clones candidates are selected for further analysis. Clones from different clusters were selected with the aim to proceed with high sequence diversity.

### Rabbit IgG Manufacturing

The rabbit IgG variable domains were cloned by RT-PCR amplification and ligation into a suitable mammalian expression vector for transient heterologous expression containing a leader sequence and the respective constant domains e.g. the pFUSE-rIgG vectors (Invivogen). The transient expression of the functional rIgG was performed by co-transfection of vectors encoding the heavy and light chains with the FreeStyle™ MAX system in CHO S cells. After cultivation for several days the supernatant of the antibody secreting cells was recovered for purification. Subsequently the secreted rabbit IgGs were affinity purified by magnetic Protein A beads (GE Healthcare). The IgG loaded beads were washed and the purified antibodies were eluted by a pH shift. The elution fractions were analyzed by sodium dodecyl sulfate polyacrylamide gel electrophoresis (SDS-PAGE), UV absorbance at 280 nm and size-exclusion high performance liquid chromatography (SE-HPLC) to ensure comparable quality of all samples.

### Engineering and Characterization of Humanized Single-Chain Fv Fragments and IgGs

The humanization of rabbit antibody clone comprised the transfer of the rabbit CDRs onto Numab's proprietary scFv acceptor framework of the VK1/VH3 type. In this process the amino acid sequence of the six CDR regions of a given rabbit clone was identified on the rabbit antibody donor sequence as described elsewhere (Borras, L. et al., J. Biol. Chem. 285 (2010) 9054-9066) and grafted into the Numab acceptor scaffold sequence. In the case of rabbit clone clone-06, for example, the VL and VH sequences of the resulting humanized clone-06 are shown in SEQ ID NO: 21 and 22, respectively. Variants of the humanized light chain are shown in SEQ ID NO: 23 and 24.

Humanized IgG constructs can be made in analogy to the method described in [00191].

### SPR Assay for Determination of Binding Kinetics and Species Cross-Reactivity of Monoclonal Anti-CD3 Antibodies

Binding affinities of monoclonal rabbit anti-CD3 antibodies were measured by surface plasmon resonance (SPR) using a MASS-1 SPR instrument (Sierra Sensors). For affinity measurements, an antibody specific for the Fc region of rabbit IgGs (Bethyl Laboratories, Cat. No. A120-111A) was immobilized on a sensor chip (SPR-2 Affinity Sensor, Amine, Sierra Sensors) using a standard amine-coupling procedure. Rabbit monoclonal antibodies were captured by the immobilized anti-rabbit IgG antibody. Two-fold serial dilutions of human heterodimeric single-chain CD3εγ extracellular domain (produced in-house) ranging from 90 to 2.81 nM were injected into the flow cells for 3 min and dissociation of the protein from the IgG captured on the sensor chip was allowed to proceed for 5 min. After each injection cycle, surfaces were regenerated with two injections of 10 mM glycine-HCl. The apparent dissociation (kd) and association (ka) rate constants and the apparent dissociation equilibrium constant (KD) were calculated with the MASS-1 analysis software (Analyzer, Sierra Sensors) using one-to-one Langmuir binding model.

### Determination of Species Cross-Reactivity

Species cross-reactivity to cynomolgus monkey single-chain CD3εγ extracellular domain was measured using the same assay setup. Three-fold serial dilutions of cynomolgus monkey heterodimeric CD3εγ extracellular domain (produced in-house) ranging from 90 to 0.12 nM were injected into the flow cells for 3 min and dissociation of the protein from the IgG captured on the sensor chip was allowed to proceed for 5 min. After each injection cycle, surfaces were regenerated with two injections of 10 mM glycine-HCl. The apparent dissociation (kd) and association (ka) rate constants and the apparent dissociation equilibrium constant (KD) were calculated with the MASS-1 analysis software (Analyzer, Sierra Sensors) using one-to-one Langmuir binding model.

### Cell-Based ELISA for Determination of Binding of Monoclonal Anti-CD3 Antibodies to CD3ε Expressed on the Cell Surface of T-Cells

Jurkat cells (clone E6-1), a human T cell line, were seeded at 300,000 cells/well in round bottom 96-well plates in 100 μl phosphate-buffered saline (PBS) containing 10% FBS. Five-fold serial dilutions of anti-CD3 rabbit monoclonal antibodies ranging from 90 nM to 0.0058 nM were added to the plates in 100 μl PBS containing 10% FBS. Binding of rabbit antibodies to CD3ε expressed on the surface of Jurkat cells was detected by a secondary antibody specifically recognizing the Fc part of rabbit antibodies of the IgG subtype (JacksonImmuno Research, Cat. No. 111-035-046). This secondary antibody was linked to the enzyme horseradish peroxidase (HRP). HRP activity was measured by addition of TMB substrate (3,3′,5,5′-tetramethylbenzidine, KPL, Cat. No. 53-00-00), which in a colorimetric reaction is processed by the HRP. The color intensity of the processed substrate is directly proportional to the amount of anti-CD3 antibody bound to Jurkat cells. To quantify color intensity, light absorbance (optical density) at the respective wave length was measured using a microtiter plate reader (Infinity reader M200 Pro, Tecan).

To correct for unspecific binding of the antibodies to unknown components presented on the cell surface of Jurkat cells, a CD3ε deficient derivative of the Jurkat T cell line (J.RT3-T3.5) was used. Binding of the monoclonal antibodies to this cell line was measured as described above for the Jurkat cells. For quantification of specific binding to Jurkat cells, the optical density for binding to the negative control was subtracted from the optical density for binding to Jurkat cells. Data were analyzed using a four-parameter logistic curve fit using the Softmax Data Analysis Software (Molecular Devices), and the molar concentration of anti-CD3 antibody required to reach 50% binding (EC₅₀, mid-OD of the standard curve) was derived from dose response curves.

### Determination of Species Cross-Reactivity

Binding to cynomolgus monkey CD3 presented on the cell surface of HSC-F T cells was measured using the same assay setup. HSC-F cells, a cynomolgus monkey T cell line, were seeded at 300,000 cells/well in round bottom 96-well plates in 100 μl phosphate-buffered saline (PBS) containing 10% FBS. Five-fold serial dilutions of anti-CD3 rabbit monoclonal antibodies ranging from 18 nM to 0.0058 nM were added to the plates in 100 μl PBS containing 10% FBS. Binding of rabbit antibodies to cynomolgus monkey CD3ε expressed on the surface of HSC-F cells was detected by a secondary antibody specifically recognizing the Fc part of rabbit antibodies of the IgG subtype (JacksonImmuno Research, Cat. No. 111-035-046). This secondary antibody was linked to the enzyme horseradish peroxidase (HRP). HRP activity was measured as described above.

To correct for unspecific binding of the antibodies to unknown components presented on the cell surface, a CD3ε negative human B lymphoblast cell line (DB) was used. Binding of the monoclonal antibodies to this cell line was measured as described above. For quantification of specific binding to HSC-F cells, the optical density for binding to the negative control was subtracted from the optical density for binding to HSC-F cells. Data were analyzed using a four-parameter logistic curve fit using the Softmax Data Analysis Software (Molecular Devices), and the molar concentration of anti-CD3 antibody required to reach 50% binding (EC₅₀, mid-OD of the standard curve) was derived from dose response curves.

### T-Cell Activation by Monoclonal Anti-CD3 Antibodies: Induction of CD69 Expression

The potential of monoclonal rabbit anti-CD3 antibodies to induce T-cell activation was evaluated by measurement of induction of CD69 expression, an early T-cell activation marker, in Jurkat cells, described elsewhere (Gil et al, Cell. 2002; 109: 901-912). For dose-response assays, Jurkat cells (100,000 cells/well) were stimulated for 24 h with 20 μg/ml, 5 μg/ml and 1.25 μg/ml of anti-CD3 antibodies. Prior to addition of anti-CD3 monoclonal antibodies to Jurkat cells, anti-CD3 antibodies were cross-linked by addition of 3-fold excess of a goat anti-rabbit IgG antibody (Bethyl Laboratories, Cat. No. A120-111A) and a rabbit anti-mouse IgG antibody (Jacksonlmmuno Research, Cat. No. 315-005-008) respectively when OKT3 (BioLegend, Cat. No. 317302) or TR66 (Novus Biologicals, Cat. No. NBP1-97446) were used. After stimulation, cells were stained for CD69 expression using a Phycoerithrin (PE)-labeled antibody specific for human CD69 (BioLegend, Cat. No. 310906) and then analyzed with a flow cytometer (FACS aria III, Becton Dickinson). As negative control unstimulated Jurkat cells incubated with the cross-linking antibody were stained with the anti-CD69 antibody described above. T-cell activation over time was assessed with a similar assay setup as described above. 100,000 Jurkat cells/well were stimulated for 0 h, 4 h, 15 h, 24 h, 48 h and 72 h with 5 μg/ml anti-CD3 antibodies that have been cross-linked as described above. Identical to the dose-response assay, CD69 expression was analyzed by flow cytometry.

Manufacturing of scDb Constructs

The nucleotide sequences encoding the various anti-IL5R×CDE3ε scDb constructs were de novo synthesized and cloned into an adapted vector for E. coli expression that is based on a pET26b(+) backbone (Novagen). The expression construct was transformed into the E. coli strain BL12 (DE3) (Novagen) and the cells were cultivated in 2YT medium (Sambrook, J., et al., Molecular Cloning: A Laboratory Manual) as a starting culture. Expression cultures were inoculated and incubated in shake flasks at 37° C. and 200 rpm. Once an OD600 nm of 1 was reached protein expression was induced by the addition of IPTG at a final concentration of 0.5 mM. After overnight expression the cells were harvested by centrifugation at 4000 g. For the preparation of inclusion bodies the cell pellet was resuspended in IB Resuspension Buffer (50 mM Tris-HCl pH 7.5, 100 mM NaCl, 5 mM EDTA, 0.5% Triton X-100). The cell slurry was supplemented with 1 mM DTT, 0.1 mg/mL Lysozyme, 10 mM Leupeptin, 100 μM PMSF and 1 μM Pepstatin. Cells are lysed by 3 cycles of ultrasonic homogenization while being cooled on ice. Subsequently 0.01 mg/mL DNAse was added and the homogenate was incubated at room temperature for 20 min. The inclusion bodies were sedimented by centrifugation at 15000 g and 4° C. The IBs were resuspended in IB resuspension Buffer and homogenized by sonication before another centrifugation. In total a minimum of 3 washing steps with IB Resuspension Buffer were performed and subsequently 2 washes with IB Wash Buffer (50 mM Tris-HCl pH 7.5, 100 mM NaCl, 5 mM EDTA) were performed to yield the final IBs.

For protein refolding the isolated IBs were resuspended in Solubilization Buffer (100 mM Tris/HCl pH 8.0, 6 M Gdn-HCl, 2 mM EDTA) in a ratio of 5 mL per g of wet IBs. The solubilization was incubated for 30 min at room temperature until DTT was added at a final concentration of 20 mM and the incubation was continued for another 30 min. After the solubilization was completed the solution was cleared by 10 min centrifugation at 21500 g and 4° C. The refolding was performed by rapid dilution at a final protein concentration of 0.3 g/L of the solubilized protein in Refolding Buffer (typically: 100 mM Tris-HCl pH 8.0, 5.0 M Urea, 5 mM Cysteine, 1 mM Cystine). The refolding reaction was routinely incubated for a minimum of 14 h. The resulting protein solution was cleared by 10 min centrifugation at 8500 g and 4° C. The refolded protein was purified by affinity chromatography on Capto L resin (GE Healthcare). The isolated monomer fraction was analyzed by size-exclusion HPLC, SDS-PAGE for purity and UV/Vis spectroscopy for protein content. Buffer was exchanged into native buffer (50 mM Citrate-Phosphate pH 6.4, 200 mM NaCl) by dialysis.

SPR Assay for Determination of Binding Kinetics of Bispecific Anti-CD3×IL5R scDbs

Binding affinities of anti-CD3×IL5R scDbs were measured by surface plasmon resonance (SPR) using a MASS-1 SPR instrument (Sierra Sensors). For affinity measurements to CD3, human heterodimeric single-chain CD3εγ extracellular domain (produced in-house) is immobilized on a sensor chip (SPR-2 Affinity Sensor High Capacity, Amine, Sierra Sensors) using a standard amine-coupling procedure. Three-fold serial dilutions of scDbs ranging from 90 to 0.1 nM were injected into the flow cells for 3 min and dissociation of the protein from the CD3εγ immobilized on the sensor chip was allowed to proceed for 12 min. After each injection cycle, surfaces are regenerated with two injections of 10 mM Glycine-HCl (pH 2.0). For affinity measurements against IL5R, an antibody specific for the Fc region of human IgGs was immobilized on a sensor chip (SPR-2 Affinity Sensor High Capacity, Amine, Sierra Sensors) by amine-coupling. A human IL5R-Fc chimeric protein (Novus Biologicals) was captured by the immobilized antibody. Three-fold serial dilutions of scDbs specific for IL5R (90 nM-0.1 nM) are injected into the flow cells for three minutes and dissociation is monitored for 12 minutes. After each injection cycle, surfaces are regenerated with three injections of 10 mM Glycine-HCl (pH 1.5). The apparent dissociation (kd) and association (ka) rate constants and the apparent dissociation equilibrium constant (K_{D}) are calculated with the MASS-1 analysis software (Analyzer, Sierra Sensors) using one-to-one Langmuir binding model.

Binding of Bispecific Anti-CD3×IL5R scDbs to CD3ε Expressed on the Cell Surface of T-Cells and to IL5R Expressed on the Surface of CHO Cells (CHO-IL5R Cells)

Binding of scDbs to CD3ε expressed on the cell surface of Jurkat cells (clone E6-1, ATCC), a human T cell line, was analyzed by flow cytometry. To assess unspecific binding of the scDbs to unknown components presented on the cell surface of Jurkat cells a CD3ε deficient derivative of the Jurkat T cell line (J.RT3-T3.5, ATCC) was used. Binding of scDbs to IL5R expressed on the cell-surface was analyzed using transgenic CHO-IL5R cells (generated at ZHAW) and wild-type CHO cells (Invitrogen) were used as controls for unspecific binding. Both cell lines were incubated with 1 μg/mL and 10 μg/mL of scDbs for 1 hour and bound scDbs were detected by addition of RPE-labeled protein L (BioVision) and then analyzed with a flow cytometer (FACS aria III, Becton Dickinson). As negative control a scFv specific for an unrelated target was used. For the qualitative assessment of binding to Jurkat and CHO-IL5R cells the mean fluorescence intensity (MFI), reflecting the signal intensity at the geometric mean, was measured for both, the unspecific scFv as well as for the test scDbs. The difference of the MFI between test antibody and negative control antibody (ΔMFI) was calculated as a measure for binding. Furthermore, the normalized MFI was calculated by dividing the MFI of the test scDb through the MFI of the negative control scFv.

T-Cell Activation by Bispecific Anti-CD3×IL5R scDbs: Induction of IL-2 Secretion

The potential of anti-CD3×anti-IL5R scDbs to induce IL-2 expression in CD8+ cytotoxic T-cells in presence of target cells was evaluated as follows. Cytotoxic T-cells were freshly isolated from human blood by using the RosetteSep™ human CD8+ T-cell enrichment cocktail (STEMCELL Technologies) according to the manufacturer's instructions. CHO-IL5R cells (10,000 cells/well) were incubated with CD8+ cytotoxic T-cells at an effector:target ratio of 10:1 in presence of 10-fold serially diluted scDbs (100 nM to 0.001 nM) in 96 well microtiter plates. To assess unspecific stimulation of T-cells wild-type CHO cells were used as target cells. Supernatant was collected after 16 hours of co-incubation to measure IL-2 release. IL-2 release was quantified using a commercially available ELISA kit (BioLegend). Data were analyzed using a four-parameter logistic curve fit using the SoftMax® Pro data analysis Software (Molecular Devices), and the molar concentration of scDb required to induce half maximal IL-2 secretion (EC₅₀) is derived from dose-response curves.

scDb Mediated Lysis of IL5R Expressing CHO Cells by Cytotoxic T Cells

For assessment of the potential of bispecific anti-CD3×IL5R scDbs to induce target cell lysis a transgenic IL5R expressing CHO cell line was used (CHO-IL5R). Unstimulated human CD8+ T-cells isolated as described above were used as effector cells. Target cells were labeled with cell tox green dye (Promega) according to the manufacturer's instructions. Cell lysis was monitored by the CellTox™ green cytotoxicity assay (Promega). The assay measures changes in membrane integrity that occur as a result of cell death. The assay uses an asymmetric cyanine dye that is excluded from viable cells but preferentially stains the dead cell DNA. When the dye binds DNA in compromised cells, its fluorescence properties are substantially enhanced. Viable cells produce no appreciable increases in fluorescence. Therefore, the fluorescence signal produced by the binding interaction with dead cell DNA is proportional to cytotoxicity. Similarly as described above, labeled CHO-IL5R cells (10,000 cells/well) were incubated with CD8+ cytotoxic T-cells at an effector:target ratio of 10:1 in presence of 10-fold serially diluted scDbs (100 nM to 0.001 nM) in 96 well microtiter plates. To assess unspecific lysis of cells that do not express the target, T-cells were co-incubated with labeled wild-type CHO cells. Fluorescence intensity was analyzed after 88 h of incubation using a multi-mode microplate reader (FlexStation 3, Molecular Devices). Data were analyzed using a four-parameter logistic curve fit using the SoftMax® Pro data analysis Software (Molecular Devices), and the molar concentration of scDb required to induce half maximal target cell lysis (EC₅₀) was derived from dose-response curves.

Engineering of scDb Constructs for Example 9

Two different single-chain diabody (scDb) constructs were engineered using well-known standard recombinant DNA techniques. Both scDbs contain identical IL5R binding variable domains (VL: SEQ ID NO: 29; VH: SEQ ID NO: 30)) but different anti-CD3 domains. The two anti-CD3 binding domains used are on one hand the humanized variable domain of clone 6 (SEQ ID NO: 21: VL; SEQ ID NO: 22: VH) and on the other hand the variable domain of the anti-CD3 antibody TR66 described elsewhere (Moore et al, Blood. 2011; 117:4542-4551).

The bispecific scDb constructs were of the following design: VLA-L1-VHB-L2-VLB-L3-VHA wherein the VLA and VHA domains jointly form the antigen binding site for human IL5R, and VLB and VHB jointly form the antigen binding site for human CD3ε. These variable domain sequence segments are linked by the flexible amino acid linkers L1 and L3 each consisting of the amino acid sequences GGGGS (G₄S) and the middle linker L2 consisting of the amino acid sequence GGGGSGGGGSGGGGSGGGGS (G₄S)₄.

Manufacturing of scDb Constructs for Example 9

The nucleotide sequences encoding the two anti-IL5R×CDE3ε scDb constructs were de novo synthesized and cloned into an adapted vector for E. coli expression that is based on a pET26b(+) backbone (Novagen). The expression construct was transformed into the E. coli strain BL12 (DE3) (Novagen) and the cells were cultivated in 2YT medium (Sambrook, J., et al., Molecular Cloning: A Laboratory Manual) as a starting culture. Expression cultures were inoculated and incubated in shake flasks at 37° C. and 200 rpm. Once an OD600 nm of 1 was reached protein expression was induced by the addition of IPTG at a final concentration of 0.5 mM. After overnight expression the cells were harvested by centrifugation at 4000 g. For the preparation of inclusion bodies the cell pellet was resuspended in IB Resuspension Buffer (50 mM Tris-HCl pH 7.5, 100 mM NaCl, 5 mM EDTA, 0.5% Triton X-100). The cell slurry was supplemented with 1 mM DTT, 0.1 mg/mL Lysozyme, 10 mM Leupeptin, 100 μM PMSF and 1 μM Pepstatin. Cells are lysed by 3 cycles of ultrasonic homogenization while being cooled on ice. Subsequently 0.01 mg/mL DNAse was added and the homogenate was incubated at room temperature for 20 min. The inclusion bodies were sedimented by centrifugation at 15000 g and 4° C. The IBs were resuspended in IB resuspension Buffer and homogenized by sonication before another centrifugation. In total a minimum of 3 washing steps with IB Resuspension Buffer were performed and subsequently 2 washes with IB Wash Buffer (50 mM Tris-HCl pH 7.5, 100 mM NaCl, 5 mM EDTA) were performed to yield the final IBs.

For protein refolding the isolated IBs were resuspended in Solubilization Buffer (100 mM Tris/HCl pH 8.0, 6 M Gdn-HCl, 2 mM EDTA) in a ratio of 5 mL per g of wet IBs. The solubilization was incubated for 30 min at room temperature until DTT was added at a final concentration of 20 mM and the incubation was continued for another 30 min. After the solubilization was completed the solution was cleared by 10 min centrifugation at 21500 g and 4° C. The refolding was performed by rapid dilution at a final protein concentration of 0.3 g/L of the solubilized protein in Refolding Buffer (typically: 100 mM Tris-HCl pH 8.0, 5.0 M Urea, 5 mM Cysteine, 1 mM Cystine). The refolding reaction was routinely incubated for a minimum of 14 h. The resulting protein solution was cleared by 10 min centrifugation at 8500 g and 4° C. The refolded protein was purified by affinity chromatography on Capto L resin (GE Healthcare). The isolated monomer fraction was analyzed by size-exclusion HPLC, SDS-PAGE for purity and UV/Vis spectroscopy for protein content. Buffer was exchanged into native buffer (50 mM Citrate-Phosphate pH 6.4, 200 mM NaCl) by dialysis.

Construct Design and Manufacturing of Surrogate scDb Construct

The single-chain diabody construct was designed by arranging the variable domains in a VLA-L1-VHB-L2-VLB-L3-VHA configuration. In these constructs the VLA and VHA domains jointly form the binding site for IL23R while the VLB and VHB domains jointly form the binding site for CD3ε. The peptide linkers L1-L3 connecting the variable domains are constructed of the glycine/serine repeats. The two short linkers L1 and L3 are composed of a single G₄S repeat, whereas the long linker L2 is composed of the sequence (G₄S)₄. The nucleotide sequences encoding the anti-IL23R×CDE3ε scDb construct was de novo synthesized and cloned into an adapted vector for mammalian expression that is based on a pcDNA3.1 backbone (Invitrogen) with an IL-2 signal sequence preceding the open reading frame. The transient expression of the functional scDb was performed with the FreeStyle™ MAX system in CHO S cells (Invitrogen). After cultivation for several days the supernatant of the expression culture was recovered for purification.

The protein was purified by affinity chromatography on Capto L resin (GE Healthcare) optionally followed by a size-exclusion chromatography on a Superdex 75 column (GE Healthcare). The proteins were formulated in PBS buffer (Lonza, REF BE17-517Q). The isolated monomer fraction was analyzed by size-exclusion HPLC, SDS-PAGE for purity and UV/Vis spectroscopy for protein content.

### Construct Design and Manufacturing of Surrogate Fab-scFv Fusion Construct and Anti-mCD3 Fab

The heterodimeric Fab-scFv construct was designed by preparing to mammalian expression constructs for a co-transfection in suitable host cells. The configuration of these protein chains are VLA-CL-L4-VLB-L2-VHB and VHA-CH1-L4-VLB-L2-VHB, respectively. In these constructs the domains VLA and VHA correspond to the native amino acid sequence from the 2C11 hamster antibody that have been combined with human CL and CH1 sequences to form a chimeric Fab fragment. The scFv modules VLB-L2-VHB from the anti-IL23R 14-11-D07-sc01 have been fused to the C-terminus of the constant domains via an intermediate linker L4 composed of the sequence (G₄S)₂. In addition to the bispecific construct also the chimeric Fab fragment of the 2C11 was constructed without the fusion of the scFv modules.

The nucleotide sequences encoding the constructs were de novo synthesized and cloned into an adapted vector for mammalian expression that is based on a pcDNA3.1 backbone (Invitrogen) with an IL-2 signal sequence preceding the open reading frame. The transient expression of the functional scDb was performed with the FreeStyle™ MAX system in CHO S cells (Invitrogen). After cultivation for several days the supernatant of the expression culture was recovered for purification.

The protein was purified by affinity chromatography on Kappa Select resin (GE Healthcare) optionally followed by a size-exclusion chromatography on a Superdex 75 column (GE Healthcare). The proteins were formulated in PBS buffer (Lonza, REF BE17-517Q). The isolated monomer fraction was analyzed by size-exclusion HPLC, SDS-PAGE for purity and UV/Vis spectroscopy for protein content.

T-Cell Activation by Bispecific Anti-CD3×IL5R scDbs for Example 9

The potential of anti-IL5R×CD3 scDbs to induce cytokine expression in CD8+ cytotoxic T-cells in presence of target cells was evaluated as follows. CD8+ T-cells were freshly isolated from human blood by using the RosetteSep™ human CD8+ T-cell enrichment cocktail (STEMCELL Technologies) according to the manufacturer's instructions or from human buffy coats using the EasySep™ Human CD8+ T Cell Enrichment Kit (STEMCELL Technologies). CHO-IL5R cells (10,000 cells/well) were incubated with CD8+ cytotoxic T-cells at an effector:target ratio of 10:1 in presence of serially diluted scDbs (100, 20, 4, 0.8, 0.16, 0.032, 0.0064 nM) in 96 well microtiter plates. To assess unspecific stimulation of T-cells wild-type CHO cells were used as target cells. Supernatant was collected after 64 hours of co-incubation to measure cytokine concentrations. Cytokine release was quantified using commercially available ELISA kits (IFNγ: BioLegend; TNF:BioLegend; IL-10: BioLegend; TGFβ: BioLegend; IL-6: BioLegend). Data were analyzed using a four-parameter logistic curve fit using the SoftMax Pro data analysis Software (Molecular Devices).

The potential of bispecific anti-IL5R×CD3 scDbs to induce T-cell activation was evaluated by measurement of induction of CD69 expression, an early T-cell activation marker, described elsewhere (Gil et al, Cell. 2002; 109: 901-912). After 18 hours, cells were stained for CD69 expression using a Phycoerithrin (PE)-labeled antibody specific for human CD69 (BioLegend, Cat. No. 310906) and then analyzed with a flow cytometer (NovoCyte, Acea Biosciences). As negative control unstimulated human CD8+ T cells were incubated with hIL5R negative CHO cells at the same conditions as described above.

Similar results can be obtained by using analogous procedures for other exhaustion markers, such as TIM-3, PD-1, CTLA-4, CD160, CD244, or LAG-3.

scDb Mediated Lysis of IL5R Expressing CHO Cells by Cytotoxic T Cells for Example 9

For assessment of the potential of bispecific anti-IL5R×CD3 scDbs to induce target cell lysis a transgenic IL5R expressing CHO cell line was used (CHO-IL5R). Unstimulated human CD8+ T-cells isolated as described above were used as effector cells. Target cells were labeled with cell tox green dye (Promega) according to the manufacturer's instructions. Cell lysis was monitored by the CellTox™ green cytotoxicity assay (Promega). The assay measures changes in membrane integrity that occur as a result of cell death. The assay uses an asymmetric cyanine dye that is excluded from viable cells but preferentially stains the dead cell DNA. When the dye binds DNA in compromised cells, its fluorescence properties are substantially enhanced. Viable cells produce no appreciable increases in fluorescence. Therefore, the fluorescence signal produced by the binding interaction with dead cell DNA is proportional to cytotoxicity. Similarly as described above, labeled CHO-IL5R cells (10,000 cells/well) were incubated with CD8+ cytotoxic T-cells at an effector:target ratio of 10:1 in presence of 5-fold serially diluted scDbs (100, 20, 4, 0.8, 0.16, 0.032, 0.0064 nM) in 96 well microtiter plates. To assess unspecific lysis of cells that do not express the target, T-cells were co-incubated with labeled wild-type CHO cells. Fluorescence intensity was analyzed after 18, 24, 40, 48 and 64 hours of incubation using a multi-mode microplate reader (FlexStation 3, Molecular Devices). Data were analyzed using a four-parameter logistic curve fit using the SoftMax Pro data analysis Software (Molecular Devices), and the molar concentration of scDb required to induce half maximal target cell lysis (EC₅₀) was derived from dose-response curves.

TABLE 4
Residues most affecting binding of the different antibodies

| Clone ID NO. | SET 1 | SET 2 | SET 3 | SET 4 |
|---|---|---|---|---|
| clone-06 | N4; E6 | N4; E6 | binding low | binding low |
| clone-02 | N4; E6 | N4; E6; (G8) | binding low | binding low |
| clone-03 | N4; E6 | N4; E6; (G8) | binding low | binding low |
| clone-04 | N4; E6 | G3; E6 | binding low | binding low |
| clone-10 | N4; E6 | N4; E6 | binding low | binding low |

TABLE 5
Sequences of anti-CD3 antibodies

| | | Heavy | |
|---|---|---|---|
| | | Chain/ | |
| SEQ | Antibody | Light | |
| ID NO. | clone | Chain | Amino acid sequence |
| 1 | clone-01 | VL | AQVLTQTASSVSAAVGGTVTISCQSSESVY |
| | | | NNNRLSWFQQKPGQPPKQLIYSASSLASG |
| | | | VPSRFKGSGSGTQFTLTISDLECDDAATYY |
| | | | CQGEFSCSSADCFTFGGGTEVVVKGD |
| 2 | clone-01 | VH | QSVEESGGRLVTPGTPLTLTCTVSGFPLSS |
| | | | YAMIWVRQAPGKGLEWIGMILRAGNIYYAS |
| | | | WAKGRFTISKTSTTVDLKITSPTTEDTATYF |
| | | | CARRQYNTDGYPIGIGDLWGPGTLVTVSS |
| 3 | clone-02 | VL | AQVLTQTPSSVSAVVGGTVTISCQSSESVY |
| | | | SNNRLSWFQQKPGQPPKLLIYSASTLASGV |
| | | | PSRFKGSGSGTQFTLTITDLECDDAATYFC |
| | | | QGEFSCSSVDCFSFGGGTEVVVKGD |
| 4 | clone-02 | VH | QSLEESGGRLVTPGTPLTLTCTVSGFPLSA |
| | | | YAMIWVRQAPGKGLEWIGMIIRSGTVYYAN |
| | | | WAKGRFTISKTSTTVDLKITSPTTEDTATYF |
| | | | CARRHYNADGYPIGIGDLWGPGTLVTVSS |
| 5 | clone-03 | VL | AQVLTQTPSSVSAAVGGTVTISCQSNENIYS |
| | | | NNRLSWFQQKPGQPPNQLIYSASSLASGV |
| | | | PSRFKGSGSGTQFTLTISDLECDDAATYYC |
| | | | QGEFNCNSADCFTFGGGTEVVVKGD |
| 6 | clone-03 | VH | QSLEESGGRLVTPGTPLTLTCTVSGFPLNR |
| | | | YAMLWVRQAPGKGLEWIGLITRADKKYYAS |
| | | | WAKGRFTISKTSTTVDLEITGPTTEDTATYF |
| | | | CARRHYNTDGYPIAIGDLWGPGTLVTVSS |
| 7 | clone-04 | VL | AQVLTQTPSSVSAAVGGTVTINCQSSQSVY |
| | | | NNNRLSWFQQKPGQPPKLLIYTTSSLASGV |
| | | | PSRFKGSGSGTEFTLTISDLECADAATYYC |
| | | | QGEFSCSRADCFNFGGGTEVVVKGD |
| 8 | clone-04 | VH | QSLEESGGRLVKPDETLTLTCTVSGFPLSS |
| | | | YAMGWFRQAPGKGLEWIGMILRSDNTYYA |
| | | | SWAKGRFTISKTSTTVDLKITSPTTEDTATY |
| | | | FCARRHYNASGNPIAIGDLWGPGTLVTVSS |
| 9 | clone-06 | VL | AQVLTQTPSSVSAAVGGTVTISCQSSESVY |
| | | | NNKRLSWFQQKPGQPPKQLIYTASSLASGV |
| | | | PSRFKGSGSGTQFTLTISDLECDDAATYYC |
| | | | QGEFTCSNADCFTFGGGTEVVVKGD |
| 10 | clone-06 | VH | QSVEESGGRLVTPGTPLTLTCTVSGFPLSS |
| | | | YAMIWVRQAPGKGLEWIGMILRAGNIYYAS |
| | | | WVKGRVTISKTSTTVDLKITSPTTEDTATYF |
| | | | CARRHYNREGYPIGIGDLWGPGTLVTVSS |
| 11 | clone-09 | VL | AQVLTQTPSSVSAAVGGTVTISCQSNENIYS |
| | | | NNRLSWFQQKPGQPPNQLIYSASSLASGV |
| | | | PSRFKGSGSGTQFTLTISDLECDDAATYYC |
| | | | QGEFNCNSADCFTFGGGTEVVVKGD |
| 12 | clone-09 | VH | QSLEESGGRLVTPGTPLTLTCTVSGFPLNR |
| | | | YAMLWVRQAPGKGLEWIGLITRADKKYYAS |
| | | | WAKGRFTISKTSTTVDLEITGPTTEDTATYF |
| | | | CARRHYNTDGYPVAIGDLWGPGTLVTVSS |
| 13 | clone-10 | VL | AQVLTQTPSSVSAAVGGTATISCQSNENIYS |
| | | | NNRLSWFQQKAGQPPNQLIYSASSLASGV |
| | | | PSRFKGSGSGTQFTLTISDLECDDAATYYC |
| | | | QGEFSCSSADCFTFGGGTEVVVKGD |
| 14 | clone-10 | VH | QSLEESGGRLVTPGTPLTLTCTVSGFPLSS |
| | | | FAMLWVRQAPGKGLEWIGMIMRAHNMYYA |
| | | | SWAKGRFTISKTSTTVDLEITSPTTEDTATY |
| | | | FCARRHYNTYGYPIAIGDLWGPGTLVTVSS |
| 15 | clone-11 | VL | AQVLTQTPSSVSAAVGGTVTINCQSSQSVY |
| | | | NNNRLSWFQQKPGQPPKLLIYTASSLASGV |
| | | | PSRFKGSGSGTEFTLTISDLECADAATYYC |
| | | | QGEFSCSSADCFTFGGGTEVVVKGD |
| 16 | clone-11 | VH | QSLEESGGRLVTPGTPLTLTCTVSGFPLSS |
| | | | YAMGWFRQAPGKGLEWIGMILRADNTYYA |
| | | | SWVNGRFTISKTSTTVDLKITSPTTEDTATY |
| | | | FCARRHYNTYGYPVAIGDLWGPGTLVTVSS |
| 17 | clone-12 | VL | AQVLTQTPSSVSATVGGTVTISCQSNENIYS |
| | | | NNRLSWFQQKPGQPPKLLIYSASSLASGVP |
| | | | SRFKGSGSGTQFTLTISDLECDDAATYYCQ |
| | | | GEFNCNSADCFTFGGGTEVVVKGD |
| 18 | clone-12 | VH | QSLEESGGRLVTPGTPLTLTCTVSGFPLSR |
| | | | YAMLWVRQAPGKGLEWIGLITRADNKYYAS |
| | | | WAKGRFTISKTSTTVDLEITSPTTEDTATYF |
| | | | CARRHYNTDGYPIAIGDLWGPGTLVTVSS |
| 19 | consensus | VL | AQVLTQTX(P/A)SSVSAX(A/V/T)VGGTX(V/A) |
| | | | TIX(S/N)CQSX(S/N)X(E/Q)X(S/N)X(V/I)YX(S/ |
| | | | N)NX(N/K)RLSWFQQKX(P/A)GQPPX(K/N)X |
| | | | (Q/L)LIYX(S/T)X(A/T)SX(S/T)LASGVPSRFK |
| | | | GSGSGTX(Q/E)FTLTIX(S/T)DLECX(D/A)DA |
| | | | ATYX(Y/F)CQGEFX(S/N/T)CX(S/N)X(S/N/R) |
| | | | X(A/V)DCFX(T/S/N)FGGGTEVVVKGD |
| 20 | consensus | VH | QSX(L/V)EESGGRLVX(T/K)PX(G/D)X(T/E)X |
| | | | (P/T)LTLTCTVSGFPLX(S/N)X(S/A/R)X(Y/F)A |
| | | | MX(L/I/G)WX(V/F)RQAPGKGLEWIGX(M/L)I |
| | | | X(L/T/M/I)RX(A/S)X(D/G/H)X(N/K/T)X(K/T/I/V/ |
| | | | M)YYAX(S/N)WX(A/V)X(K/N)GRX(F/V)TISK |
| | | | TSTTVDLX(K/E)ITX(S/G)PTTEDTATYFCAR |
| | | | RX(H/Q)YNX(T/A/R)X(D/Y/S/E)GX(Y/N)PX(I/ |
| | | | V)X(A/G)IGDLWGPGTLVTVSS |
| 21 | humanized | VL | DIQMTQSPSSLSASVGDRVTITCQSSESVY |
| | clone-06; | | NNKRLSWYQQKPGKAPKLLIYTASSLASGV |
| | variant A | | PSRFSGSGSGTDFTLTISSLQPEDFATYYC |
| | | | QGEFTCSNADCFTFGQGTKLTVLG |
| 22 | humanized | VH | EVQLVESGGGLVQPGGSLRLSCAASGFPL |
| | clone-06 | | SSYAMIWVRQAPGKGLEWIGMILRAGNIYY |
| | | | ASWVKGRFTISRDNSKNTVYLQMNSLRAED |
| | | | TAVYYCARRHYNREGYPIGIGDLWGQGTLV |
| | | | TVSS |
| 23 | humanized | VL | DIQMTQSPSSLSASVGDRVTITCQSSESVY |
| | clone-06; | | NNKRLSWYQQKPGKAPKLLIYTASSLASGV |
| | variant B | | PSRFSGSGSGTDFTLTISSLQPEDFATYYC |
| | | | QGEFTCSNADCFTFGGGTKLTVLG |
| 24 | humanized | VL | DIQMTQSPSSLSASVGDRVTITCQSSESVY |
| | clone-06; | | NNKRLSWYQQKPGKAPKLLIYTASSLASGV |
| | variant C | | PSRFSGSGSGTDFTLTISSLQPEDFATYYC |
| | | | QGEFTCSNADCFTFGTGTKVTVLG |

[CDR1 to 3 shown in bold and underlined in SEQ ID NOs: 1 and 2 as representatives for all sequences]
[in SEQ ID NOs: 19 and 20: positions “X” are degenerate positions: respective degeneracy provided in square brackets behind individual “X”]

TABLE 6
Sequences of anti-IL23R antibodies

| | | Heavy | |
|---|---|---|---|
| SEQ | | Chain/ | |
| ID | Antibody | Light | |
| NO. | clone | Chain | Amino acid sequence |
| 25 | humanized | VL | DIQMTQSPSSLSASVGDRVTITCQASENIYS |
| | clone-01, | | FLAWYQQKPGKAPKLLIYSASKLAAGVPSR |
| | variant A | | FSGSGSGTDFTLTISSLQPEDFATYYCQQT |
| | | | NRYSNPDIYNVFGQGTKLTVLG |
| 26 | humanized | VL | DIQMTQSPSSLSASVGDRVTITCQASENIYS |
| | clone-01, | | FLAWYQQKPGKAPKLLIYSASKLAAGVPSR |
| | variant B | | FSGSGSGTDFTLTISSLQPEDFATYYCQQT |
| | | | NRYSNPDIYNVFGGGTKLTVLG |
| 27 | humanized | VL | DIQMTQSPSSLSASVGDRVTITCQASENIYS |
| | clone-01, | | FLAWYQQKPGKAPKLLIYSASKLAAGVPSR |
| | variant C | | FSGSGSGTDFTLTISSLQPEDFATYYCQQT |
| | | | NRYSNPDIYNVFGTGTKVTVLG |
| 28 | humanized | VH | EVQLVESGGGLVQPGGSLRLSCAASGIDFN |
| | clone-01 | | SNYYMCWVRQAPGKGLEWIGCIYVGSHVN |
| | | | TYYANWAKGRFTISRDNSKNTVYLQMNSLR |
| | | | AEDTAVYYCATSGSSVLYFKFWGQGTLVTV |
| | | | SS |

TABLE 7
Sequences of anti-IL5R antibodies

| | | Heavy | |
|---|---|---|---|
| SEQ | | Chain/ | |
| ID | Antibody | Light | |
| NO. | clone | Chain | Amino acid sequence |
| 29 | clone-01 | VL | DIQMTQSPSSLSASVGDRVTITCQASQNIYS |
| | | | NLAWYQQKPGKAPKLLIYRASTLASGVPSR |
| | | | FSGSGSGTDFTLTISSLQPEDFATYYCQSN |
| | | | YGINYYGAAFGQGTKLTVLG |
| 30 | clone-01 | VH | EVQLVESGGGLVQPGGSLRLSCAASGFSL |
| | | | SSYDMTWVRQAPGKGLEWIGIIYVSGSAYY |
| | | | ASWAKGRFTISRDNSKNTVYLQMNSLRAED |
| | | | TAVYYCARINYGLDLWGQGTLVTVSS |

TABLE 8
Sequence listing for PRO165:
VLA-L1-VHB-L2-VLB-L3-VHA

| SEQ | | |
|---|---|---|
| ID | Type | Sequence |
| 31 | Linker L1 | GGGGS |
| 32 | Linker L2 | GGGGS GGGGS GGGGS GGGGS |
| | Linker L3 | GGGGS |
| 33 | VL | DIQMTQSPSSLSASVGDRVTITCQASENIYSFLA |
| | anti-IL23R | WYQQKPGKAPKLLIYSASKLAAGVPSRFSGSGS |
| | 14-11-D07- | GTDFTLTISSLQPEDFATYYCQQTNRYSNPDIYN |
| | sc01 | VFGQGTKLTVLG |
| 34 | VH | EVQLVESGGGLVQPGGSLRLSCAASGIDFNSNY |
| | anti-IL23R | YMCWVRQAPGKGLEWIGCIYVGSHVNTYYANW |
| | 14-11-D07- | AKGRFTISRDNSKNTVYLQMNSLRAEDTAVYYC |
| | sc01 | ATSGSSVLYFKFWGQGTLVTVSS |
| 35 | VL | DIQMTQSPSSLSASVGDRVTITCQSSESVYNNKR |
| | anti-CD3 | LSWYQQKPGKAPKLLIYTASSLASGVPSRFSGS |
| | clone 6 | GSGTDFTLTISSLQPEDFATYYCQGEFTCSNADC |
| | | FTFGQGTKLTVLG |
| 36 | VH | EVQLVESGGGLVQPGGSLRLSCAASGFPLSSYA |
| | anti-CD3 | MIWVRQAPGKGLEWIGMILRAGNIYYASWVKGR |
| | clone 6 | FTISRDNSKNTVYLQMNSLRAEDTAVYYCARRH |
| | | YNREGYPIGIGDLWGQGTLVTVSS |
| 37 | PRO165 | DIQMTQSPSSLSASVGDRVTITCQASENIYSFLA |
| | | WYQQKPGKAPKLLIYSASKLAAGVPSRFSGSGS |
| | | GTDFTLTISSLQPEDFATYYCQQTNRYSNPDIYN |
| | | VFGQGTKLTVLGGGGGSEVQLVESGGGLVQPG |
| | | GSLRLSCAASGFPLSSYAMIWVRQAPGKGLEWI |
| | | GMILRAGNIYYASWVKGRFTISRDNSKNTVYLQM |
| | | NSLRAEDTAVYYCARRHYNREGYPIGIGDLWGQ |
| | | GTLVTVSSGGGGSGGGGSGGGGSGGGGSDIQ |
| | | MTQSPSSLSASVGDRVTITCQSSESVYNNKRLS |
| | | WYQQKPGKAPKLLIYTASSLASGVPSRFSGSGS |
| | | GTDFTLTISSLQPEDFATYYCQGEFTCSNADCFT |
| | | FGQGTKLTVLGGGGGSEVQLVESGGGLVQPGG |
| | | SLRLSCAASGIDFNSNYYMCWVRQAPGKGLEWI |
| | | GCIYVGSHVNTYYANWAKGRFTISRDNSKNTVYL |
| | | QMNSLRAEDTAVYYCATSGSSVLYFKFWGQGTL |
| | | VTVSS |
| 38 | VL | DIQMTQSPSSLPASLGDRVTINCQASQDISNYLN |
| | anti-mCD3 | WYQQKPGKAPKLLIYYTNKLADGVPSRFSGSGS |
| | 2C11 | GRDSSFTISSLESEDIGSYYCQQYYNYPWTFGP |
| | | GTKLEIKR |
| 39 | VH | EVQLVESGGGLVQPGKSLKLSCEASGFTFSGYG |
| | anti-mCD3 | MHWVRQAPGRGLESVAYITSSSINIKYADAVKGR |
| | 2C11 | FTVSRDNAKNLLFLQMNILKSEDTAMYYCARFD |
| | | WDKNYWGQGTMVTVSS |
| 40 | PRO387 | DIQMTQSPSSLSASVGDRVTITCQASENIYSFLA |
| | | WYQQKPGKAPKLLIYSASKLAAGVPSRFSGSGS |
| | | GTDFTLTISSLQPEDFATYYCQQTNRYSNPDIYN |
| | | VFGQGTKLTVLGGGGGSEVQLVESGGGLVQPG |
| | | KSLKLSCEASGFTFSGYGMHWVRQAPGRGLES |
| | | VAYITSSSINIKYADAVKGRFTVSRDNAKNLLFLQ |
| | | MNILKSEDTAMYYCARFDWDKNYWGQGTMVTV |
| | | SSGGGGSGGGGSGGGGSGGGGSDIQMTQSPS |
| | | SLPASLGDRVTINCQASQDISNYLNWYQQKPGK |
| | | APKLLIYYTNKLADGVPSRFSGSGSGRDSSFTIS |
| | | SLESEDIGSYYCQQYYNYPWTFGPGTKLEIKRG |
| | | GGGSEVQLVESGGGLVQPGGSLRLSCAASGIDF |
| | | NSNYYMCWVRQAPGKGLEWIGCIYVGSHVNTY |
| | | YANWAKGRFTISRDNSKNTVYLQMNSLRAEDTA |
| | | VYYCATSGSSVLYFKFWGQGTLVTVSS |
| 41 | Linker L4 | GGGGS GGGGS |
| 42 | PRO386 (CL | DIQMTQSPSSLPASLGDRVTINCQASQDISNYLN |
| | fusion) | WYQQKPGKAPKLLIYYTNKLADGVPSRFSGSGS |
| | | GRDSSFTISSLESEDIGSYYCQQYYNYPWTFGP |
| | | GTKLEIKRRTVAAPSVFIFPPSDEQLKSGTASVVC |
| | | LLNNFYPREAKVQWKVDNALQSGNSQESVTEQ |
| | | DSKDSTYSLSSTLTLSKADYEKHKVYACEVTHQG |
| | | LSSPVTKSFNRGECGGGGSGGGGSHMDIQMTQ |
| | | SPSSLSASVGDRVTITCQASENIYSFLAWYQQKP |
| | | GKAPKLLIYSASKLAAGVPSRFSGSGSGTDFTLTI |
| | | SSLQPEDFATYYCQQTNRYSNPDIYNVFGQGTK |
| | | LTVLGGGGGSGGGGSGGGGSGGGGSEVQLVE |
| | | SGGGLVQPGGSLRLSCAASGIDFNSNYYMCWV |
| | | RQAPGKGLEWIGCIYVGSHVNTYYANWAKGRFT |
| | | ISRDNSKNTVYLQMNSLRAEDTAVYYCATSGSS |
| | | VLYFKFWGQGTLVTVSS |
| 43 | PRO386 (CH1 | EVQLVESGGGLVQPGKSLKLSCEASGFTFSGYG |
| | fusion) | MHWVRQAPGRGLESVAYITSSSINIKYADAVKGR |
| | | FTVSRDNAKNLLFLQMNILKSEDTAMYYCARFD |
| | | WDKNYWGQGTMVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYIC |
| | | NVNHKPSNTKVDKKVEPKSCGGGGSGGGGSH |
| | | MDIQMTQSPSSLSASVGDRVTITCQASENIYSFL |
| | | AWYQQKPGKAPKLLIYSASKLAAGVPSRFSGSG |
| | | SGTDFTLTISSLQPEDFATYYCQQTNRYSNPDIY |
| | | NVFGQGTKLTVLGGGGGSGGGGSGGGGSGGG |
| | | GSEVQLVESGGGLVQPGGSLRLSCAASGIDFNS |
| | | NYYMCWVRQAPGKGLEWIGCIYVGSHVNTYYA |
| | | NWAKGRFTISRDNSKNTVYLQMNSLRAEDTAVY |
| | | YCATSGSSVLYFKFWGQGTLVTVSS |
| 44 | PRO400 | DIQMTQSPSSLPASLGDRVTINCQASQDISNYLN |
| | (VL-CL) | WYQQKPGKAPKLLIYYTNKLADGVPSRFSGSGS |
| | 2C11 Fab | GRDSSFTISSLESEDIGSYYCQQYYNYPWTFGP |
| | | GTKLEIKRRTVAAPSVFIFPPSDEQLKSGTASVVC |
| | | LLNNFYPREAKVQWKVDNALQSGNSQESVTEQ |
| | | DSKDSTYSLSSTLTLSKADYEKHKVYACEVTHQG |
| | | LSSPVTKSFNRGEC |
| 45 | PRO400 | EVQLVESGGGLVQPGKSLKLSCEASGFTFSGYG |
| | (VH-CH1) | MHWVRQAPGRGLESVAYITSSSINIKYADAVKGR |
| | 2C11 Fab | FTVSRDNAKNLLFLQMNILKSEDTAMYYCARFD |
| | | WDKNYWGQGTMVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYIC |
| | | NVNHKPSNTKVDKKVEPKSC |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

1. A multifunctional molecule for use in the treatment of multiple sclerosis after the onset of an exacerbation episode, wherein said multifunctional molecule comprises at least (i) a target-binding moiety, which is specific for IL23R; and (ii) a second functional moiety, which leads to the depletion of IL23R-expressing cells.

2. The multifunctional molecule for use in the treatment of multiple sclerosis after the onset of an exacerbation episode according to claim 1, wherein the cell presenting the target for said target-binding-moiety is a pathogenic cell, particularly a cell selected from the group consisting of (i) a T cell expressing the transcription factor RORγ(t), (ii) a T cell producing GM-CSF and/or IFN gamma, and/or IL-17, particularly an IL-17 producing T cell (Th17 cell), (iii) a γδ T cell, (iv) a natural killer T (NKT) cell, and (v) an invariant natural killer (iNK) cell; particularly a Th17 cell or a γδ T cell.

3. The multifunctional molecule for use in the treatment of multiple sclerosis after the onset of an exacerbation episode according to claim 1, wherein said second functional moiety specifically binds to a first antigen present on a cytotoxic effector T (Tc) cell, particularly wherein said Tc cell is a stimulated or an unstimulated Tc cell.

4. The multifunctional molecule for use in the treatment of multiple sclerosis after the onset of an exacerbation episode according to claim 3, wherein said second functional moiety specifically binds to an antigen selected from CD3 and CD28.

5. The multifunctional molecule for use in the treatment of multiple sclerosis after the onset of an exacerbation episode according to claim 4, said second functional moiety is a binding molecule comprising a binding region that is specific for an epitope of human CD3, particularly for an epitope of the epsilon chain of human CD3 (CD3ε), more particularly to an agonistic epitope of CD3ε.

6. The multifunctional molecule for use in the treatment of multiple sclerosis after the onset of an exacerbation episode according to claim 5, wherein said epitope comprises amino acid residue N4 as residue that is critical for binding, particularly wherein said epitope further comprises amino acid residue E6 as residue that is involved in binding; and/or wherein at least one of residues Q1, D2, G3 and E5 of human CD3e is non-critical for binding.

7. The multifunctional molecule for use in the treatment of multiple sclerosis after the onset of an exacerbation episode according to claim 6, wherein said binding region is an antibody or a functional fragment thereof comprising an antigen-binding region comprising a VL domain selected from the group of SEQ ID NOs: 21, 23, and 24, and the VH domain of SEQ ID NO: 22; or an antigen-binding region comprising the VL domain of SEQ ID NO: 35, and the VH domain of SEQ ID NO: 36.

8. The multifunctional molecule for use in the treatment of multiple sclerosis after the onset of an exacerbation episode according to claim 1, wherein said target-binding moiety is an antibody or a functional fragment thereof comprising an antigen-binding region comprising a VL domain selected from the group of SEQ ID NOs: 25, 26, and 27, and the VH domain of SEQ ID NO: 28, or an antigen-binding region comprising the VL domain of SEQ ID NO: 33, and the VH domain of SEQ ID NO: 34.

9. The multifunctional molecule for use in the treatment of multiple sclerosis after the onset of an exacerbation episode according to claim 1, wherein said multifunctional molecule comprises the single-chain fragment of SEQ ID NO: 37.

10. The multifunctional molecule for use in the treatment of multiple sclerosis after the onset of an exacerbation episode according to claim 1, wherein said second functional moiety specifically binds to an Fc receptor, in particular to an Fc gamma receptor (FcγR), in particular to (i) an FcγRIII present on the surface of natural killer (NK) cells or (ii) one of FcγRI, FcγRIIA, FcγRIIB1, FcγRIIB2, and FcγRIIIB present on the surface of macrophages, monocytes, neutrophils and/or dendritic cells.

11. The multifunctional molecule for use in the treatment of multiple sclerosis after the onset of an exacerbation episode according to claim 1, wherein said patient does not respond to treatment with antagonists of cytokines that are involved in the differentiation of (i) T cells expressing the transcription factor RORγ(t), (ii) T cells producing GM-CSF and/or IFN gamma, and/or IL-17, particularly an IL-17 producing T cells (Th17 cells), (iii) γδ T cells, (iv) a natural killer T (NKT) cells, and (v) invariant natural killer (iNK) cells; particularly a Th17 cells or a γδ T cells.

12. The multifunctional molecule for use in the treatment of multiple sclerosis after the onset of an exacerbation episode according to claim 10, wherein said patient does not respond to treatment with IL-23 antagonists.

13. The multifunctional molecule for use in the treatment of multiple sclerosis after the onset of an exacerbation episode according to claim 1, wherein said exacerbation episode is a clinically isolated syndrome.

14. The multifunctional molecule for use in the treatment of multiple sclerosis after the onset of an exacerbation episode according to claim 1, wherein said multiple sclerosis is a progressive form of multiple sclerosis, in particular a progressive form of multiple sclerosis accompanied by systemic inflammation.

15. The multifunctional molecule for use in the treatment of multiple sclerosis after the onset of an exacerbation episode according to claim 1, wherein said exacerbation episode is an acute phase of a neuromyelitis optica.

16. The multifunctional molecule for use in the treatment of multiple sclerosis after the onset of an exacerbation episode according to claim 1, wherein said exacerbation episode is an acute phase of Asian multiple sclerosis.


SEQUENCE LISTING (157 sequences)

SEQ ID NO: 1 - protein, 115 aa, Artificial Sequence
  artificial antibody variable chain
AQVLTQTASSVSAAVGGTVTISCQSSESVYNNNRLSWFQQKPGQPPKQLIYSASSLASGVPSRFKGSGSGTQFTLTISDLECDDAATYYCQGEFSCSSADCFTFGGGTEVVVKGD

SEQ ID NO: 2 - protein, 120 aa, Artificial Sequence
  artificial antibody variable chain
QSVEESGGRLVTPGTPLTLTCTVSGFPLSSYAMIWVRQAPGKGLEWIGMILRAGNIYYASWAKGRFTISKTSTTVDLKITSPTTEDTATYFCARRQYNTDGYPIGIGDLWGPGTLVTVSS

SEQ ID NO: 3 - protein, 115 aa, Artificial Sequence
  artificial antibody variable chain
AQVLTQTPSSVSAVVGGTVTISCQSSESVYSNNRLSWFQQKPGQPPKLLIYSASTLASGVPSRFKGSGSGTQFTLTITDLECDDAATYFCQGEFSCSSVDCFSFGGGTEVVVKGD

SEQ ID NO: 4 - protein, 120 aa, Artificial Sequence
  artificial antibody variable chain
QSLEESGGRLVTPGTPLTLTCTVSGFPLSAYAMIWVRQAPGKGLEWIGMIIRSGTVYYANWAKGRFTISKTSTTVDLKITSPTTEDTATYFCARRHYNADGYPIGIGDLWGPGTLVTVSS

SEQ ID NO: 5 - protein, 115 aa, Artificial Sequence
  artificial antibody variable chain
AQVLTQTPSSVSAAVGGTVTISCQSNENIYSNNRLSWFQQKPGQPPNQLIYSASSLASGVPSRFKGSGSGTQFTLTISDLECDDAATYYCQGEFNCNSADCFTFGGGTEVVVKGD

SEQ ID NO: 6 - protein, 120 aa, Artificial Sequence
  artificial antibody variable chain
QSLEESGGRLVTPGTPLTLTCTVSGFPLNRYAMLWVRQAPGKGLEWIGLITRADKKYYASWAKGRFTISKTSTTVDLEITGPTTEDTATYFCARRHYNTDGYPIAIGDLWGPGTLVTVSS

SEQ ID NO: 7 - protein, 115 aa, Artificial Sequence
  artificial antibody variable chain
AQVLTQTPSSVSAAVGGTVTINCQSSQSVYNNNRLSWFQQKPGQPPKLLIYTTSSLASGVPSRFKGSGSGTEFTLTISDLECADAATYYCQGEFSCSRADCFNFGGGTEVVVKGD

SEQ ID NO: 8 - protein, 120 aa, Artificial Sequence
  artificial antibody variable chain
QSLEESGGRLVKPDETLTLTCTVSGFPLSSYAMGWFRQAPGKGLEWIGMILRSDNTYYASWAKGRFTISKTSTTVDLKITSPTTEDTATYFCARRHYNASGNPIAIGDLWGPGTLVTVSS

SEQ ID NO: 9 - protein, 115 aa, Artificial Sequence
  artificial antibody variable chain
AQVLTQTPSSVSAAVGGTVTISCQSSESVYNNKRLSWFQQKPGQPPKQLIYTASSLASGVPSRFKGSGSGTQFTLTISDLECDDAATYYCQGEFTCSNADCFTFGGGTEVVVKGD

SEQ ID NO: 10 - protein, 120 aa, Artificial Sequence
  artificial antibody variable chain
QSVEESGGRLVTPGTPLTLTCTVSGFPLSSYAMIWVRQAPGKGLEWIGMILRAGNIYYASWVKGRVTISKTSTTVDLKITSPTTEDTATYFCARRHYNREGYPIGIGDLWGPGTLVTVSS

SEQ ID NO: 11 - protein, 115 aa, Artificial Sequence; identical to SEQ ID NO: 5
  artificial antibody variable chain

SEQ ID NO: 12 - protein, 120 aa, Artificial Sequence
  artificial antibody variable chain
QSLEESGGRLVTPGTPLTLTCTVSGFPLNRYAMLWVRQAPGKGLEWIGLITRADKKYYASWAKGRFTISKTSTTVDLEITGPTTEDTATYFCARRHYNTDGYPVAIGDLWGPGTLVTVSS

SEQ ID NO: 13 - protein, 115 aa, Artificial Sequence
  artificial antibody variable chain
AQVLTQTPSSVSAAVGGTATISCQSNENIYSNNRLSWFQQKAGQPPNQLIYSASSLASGVPSRFKGSGSGTQFTLTISDLECDDAATYYCQGEFSCSSADCFTFGGGTEVVVKGD

SEQ ID NO: 14 - protein, 120 aa, Artificial Sequence
  artificial antibody variable chain
QSLEESGGRLVTPGTPLTLTCTVSGFPLSSFAMLWVRQAPGKGLEWIGMIMRAHNMYYASWAKGRFTISKTSTTVDLEITSPTTEDTATYFCARRHYNTYGYPIAIGDLWGPGTLVTVSS

SEQ ID NO: 15 - protein, 115 aa, Artificial Sequence
  artificial antibody variable chain
AQVLTQTPSSVSAAVGGTVTINCQSSQSVYNNNRLSWFQQKPGQPPKLLIYTASSLASGVPSRFKGSGSGTEFTLTISDLECADAATYYCQGEFSCSSADCFTFGGGTEVVVKGD

SEQ ID NO: 16 - protein, 120 aa, Artificial Sequence
  artificial antibody variable chain
QSLEESGGRLVTPGTPLTLTCTVSGFPLSSYAMGWFRQAPGKGLEWIGMILRADNTYYASWVNGRFTISKTSTTVDLKITSPTTEDTATYFCARRHYNTYGYPVAIGDLWGPGTLVTVSS

SEQ ID NO: 17 - protein, 115 aa, Artificial Sequence
  artificial antibody variable chain
AQVLTQTPSSVSATVGGTVTISCQSNENIYSNNRLSWFQQKPGQPPKLLIYSASSLASGVPSRFKGSGSGTQFTLTISDLECDDAATYYCQGEFNCNSADCFTFGGGTEVVVKGD

SEQ ID NO: 18 - protein, 120 aa, Artificial Sequence
  artificial antibody variable chain
QSLEESGGRLVTPGTPLTLTCTVSGFPLSRYAMLWVRQAPGKGLEWIGLITRADNKYYASWAKGRFTISKTSTTVDLEITSPTTEDTATYFCARRHYNTDGYPIAIGDLWGPGTLVTVSS

SEQ ID NO: 19 - protein, 115 aa, Artificial Sequence
  consensus sequence for antibody variable chain
  MISC_FEATURE (8)..(8): X/Xaa represents position with limited diversity, with amino acid residue selected from the group of P and A
  MISC_FEATURE (14)..(14): X/Xaa represents position with limited diversity, with amino acid residue selected from the group of A, V and T
  MISC_FEATURE (19)..(19): X/Xaa represents position with limited diversity, with amino acid residue selected from the group of V and A
  MISC_FEATURE (22)..(22): X/Xaa represents position with limited diversity, with amino acid residue selected from the group of S and N
  MISC_FEATURE (26)..(26): X/Xaa represents position with limited diversity, with amino acid residue selected from the group of S and N
  MISC_FEATURE (27)..(27): X/Xaa represents position with limited diversity, with amino acid residue selected from the group of E and Q
  MISC_FEATURE (28)..(28): X/Xaa represents position with limited diversity, with amino acid residue selected from the group of S and N
  MISC_FEATURE (29)..(29): X/Xaa represents position with limited diversity, with amino acid residue selected from the group of V and I
  MISC_FEATURE (31)..(31): X/Xaa represents position with limited diversity, with amino acid residue selected from the group of S and N
  MISC_FEATURE (33)..(33): X/Xaa represents position with limited diversity, with amino acid residue selected from the group of N and K
  MISC_FEATURE (42)..(42): X/Xaa represents position with limited diversity, with amino acid residue selected from the group of P and A
  MISC_FEATURE (47)..(47): X/Xaa represents position with limited diversity, with amino acid residue selected from the group of K and N
  MISC_FEATURE (48)..(48): X/Xaa represents position with limited diversity, with amino acid residue selected from the group of Q and L
  MISC_FEATURE (52)..(52): X/Xaa represents position with limited diversity, with amino acid residue selected from the group of S and T
  MISC_FEATURE (53)..(53): X/Xaa represents position with limited diversity, with amino acid residue selected from the group of A and T
  MISC_FEATURE (55)..(55): X/Xaa represents position with limited diversity, with amino acid residue selected from the group of S and T
  MISC_FEATURE (72)..(72): X/Xaa represents position with limited diversity, with amino acid residue selected from the group of Q and E
  MISC_FEATURE (78)..(78): X/Xaa represents position with limited diversity, with amino acid residue selected from the group of S and T
  MISC_FEATURE (83)..(83): X/Xaa represents position with limited diversity, with amino acid residue selected from the group of D and A
  MISC_FEATURE (89)..(89): X/Xaa represents position with limited diversity, with amino acid residue selected from the group of Y and F
  MISC_FEATURE (95)..(95): X/Xaa represents position with limited diversity, with amino acid residue selected from the group of S, N and T
  MISC_FEATURE (97)..(97): X/Xaa represents position with limited diversity, with amino acid residue selected from the group of S and N
  MISC_FEATURE (98)..(98): X/Xaa represents position with limited diversity, with amino acid residue selected from the group of S, N and R
  MISC_FEATURE (99)..(99): X/Xaa represents position with limited diversity, with amino acid residue selected from the group of A and V
  MISC_FEATURE (103)..(103): X/Xaa represents position with limited diversity, with amino acid residue selected from the group of T, S and N
AQVLTQTXSSVSAXVGGTXTIXCQSXXXXYXNXRLSWFQQKXGQPPXXLIYXXSXLASGVPSRFKGSGSGTXFTLTIXDLECXDAATYXCQGEFXCXXXDCFXFGGGTEVVVKGD

SEQ ID NO: 20 - protein, 120 aa, Artificial Sequence
  consensus sequence for antibody variable chain
  MISC_FEATURE (3)..(3): X/Xaa represents position with limited diversity, with amino acid residue selected from the group of L and V
  MISC_FEATURE (12)..(12): X/Xaa represents position with limited diversity, with amino acid residue selected from the group of T and K
  MISC_FEATURE (14)..(14): X/Xaa represents position with limited diversity, with amino acid residue selected from the group of G and D
  MISC_FEATURE (15)..(15): X/Xaa represents position with limited diversity, with amino acid residue selected from the group of T and E
  MISC_FEATURE (16)..(16): X/Xaa represents position with limited diversity, with amino acid residue selected from the group of P and T
  MISC_FEATURE (29)..(29): X/Xaa represents position with limited diversity, with amino acid residue selected from the group of S and N
  MISC_FEATURE (30)..(30): X/Xaa represents position with limited diversity, with amino acid residue selected from the group of S, A and R
  MISC_FEATURE (31)..(31): X/Xaa represents position with limited diversity, with amino acid residue selected from the group of Y and F
  MISC_FEATURE (34)..(34): X/Xaa represents position with limited diversity, with amino acid residue selected from the group of L, I and G
  MISC_FEATURE (36)..(36): X/Xaa represents position with limited diversity, with amino acid residue selected from the group of V and F
  MISC_FEATURE (49)..(49): X/Xaa represents position with limited diversity, with amino acid residue selected from the group of M and L
  MISC_FEATURE (51)..(51): X/Xaa represents position with limited diversity, with amino acid residue selected from the group of L, T, M and I
  MISC_FEATURE (53)..(53): X/Xaa represents position with limited diversity, with amino acid residue selected from the group of A and S
  MISC_FEATURE (54)..(54): X/Xaa represents position with limited diversity, with amino acid residue selected from the group of D, G and H
  MISC_FEATURE (55)..(55): X/Xaa represents position with limited diversity, with amino acid residue selected from the group of N, K and T
  MISC_FEATURE (56)..(56): X/Xaa represents position with limited diversity, with amino acid residue selected from the group of K, T, I, V and M
  MISC_FEATURE (60)..(60): X/Xaa represents position with limited diversity, with amino acid residue selected from the group of S and N
  MISC_FEATURE (62)..(62): X/Xaa represents position with limited diversity, with amino acid residue selected from the group of A and V
  MISC_FEATURE (63)..(63): X/Xaa represents position with limited diversity, with amino acid residue selected from the group of K and N
  MISC_FEATURE (66)..(66): X/Xaa represents position with limited diversity, with amino acid residue selected from the group of F and V
  MISC_FEATURE (78)..(78): X/Xaa represents position with limited diversity, with amino acid residue selected from the group of K and E
  MISC_FEATURE (81)..(81): X/Xaa represents position with limited diversity, with amino acid residue selected from the group of S and G
  MISC_FEATURE (96)..(96): X/Xaa represents position with limited diversity, with amino acid residue selected from the group of H and Q
  MISC_FEATURE (99)..(99): X/Xaa represents position with limited diversity, with amino acid residue selected from the group of T, A and R
  MISC_FEATURE (100)..(100): X/Xaa represents position with limited diversity, with amino acid residue selected from the group of D, Y, S and E
  MISC_FEATURE (102)..(102): X/Xaa represents position with limited diversity, with amino acid residue selected from the group of Y and N
  MISC_FEATURE (104)..(104): X/Xaa represents position with limited diversity, with amino acid residue selected from the group of I and V
  MISC_FEATURE (105)..(105): X/Xaa represents position with limited diversity, with amino acid residue selected from the group of A and G
QSXEESGGRLVXPXXXLTLTCTVSGFPLXXXAMXWXRQAPGKGLEWIGXIXRXXXXYYAXWXXGRXTISKTSTTVDLXITXPTTEDTATYFCARRXYNXXGXPXXIGDLWGPGTLVTVSS

SEQ ID NO: 21 - protein, 114 aa, Artificial Sequence
  artificial antibody variable chain
DIQMTQSPSSLSASVGDRVTITCQSSESVYNNKRLSWYQQKPGKAPKLLIYTASSLASGVPSRFSGSGSGTDFTLTISSLQPEDFATYYCQGEFTCSNADCFTFGQGTKLTVLG

SEQ ID NO: 22 - protein, 123 aa, Artificial Sequence
  artificial antibody variable chain
EVQLVESGGGLVQPGGSLRLSCAASGFPLSSYAMIWVRQAPGKGLEWIGMILRAGNIYYASWVKGRFTISRDNSKNTVYLQMNSLRAEDTAVYYCARRHYNREGYPIGIGDLWGQGTLVTVSS

SEQ ID NO: 23 - protein, 114 aa, Artificial Sequence
  artificial antibody variable chain
DIQMTQSPSSLSASVGDRVTITCQSSESVYNNKRLSWYQQKPGKAPKLLIYTASSLASGVPSRFSGSGSGTDFTLTISSLQPEDFATYYCQGEFTCSNADCFTFGGGTKLTVLG

SEQ ID NO: 24 - protein, 114 aa, Artificial Sequence
  artificial antibody variable chain
DIQMTQSPSSLSASVGDRVTITCQSSESVYNNKRLSWYQQKPGKAPKLLIYTASSLASGVPSRFSGSGSGTDFTLTISSLQPEDFATYYCQGEFTCSNADCFTFGTGTKVTVLG

SEQ ID NO: 25 - protein, 113 aa, Artificial Sequence
  artificial antibody variable chain
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLG

SEQ ID NO: 26 - protein, 113 aa, Artificial Sequence
  artificial antibody variable chain
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGGGTKLTVLG

SEQ ID NO: 27 - protein, 113 aa, Artificial Sequence
  artificial antibody variable chain
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGTGTKVTVLG

SEQ ID NO: 28 - protein, 121 aa, Artificial Sequence
  artificial antibody variable chain
EVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 29 - protein, 111 aa, Artificial Sequence
  artificial antibody variable chain
DIQMTQSPSSLSASVGDRVTITCQASQNIYSNLAWYQQKPGKAPKLLIYRASTLASGVPSRFSGSGSGTDFTLTISSLQPEDFATYYCQSNYGINYYGAAFGQGTKLTVLG

SEQ ID NO: 30 - protein, 115 aa, Artificial Sequence
  artificial antibody variable chain
EVQLVESGGGLVQPGGSLRLSCAASGFSLSSYDMTWVRQAPGKGLEWIGIIYVSGSAYYASWAKGRFTISRDNSKNTVYLQMNSLRAEDTAVYYCARINYGLDLWGQGTLVTVSS

SEQ ID NO: 31 - protein, 5 aa, Artificial Sequence
  artificial linker sequence
GGGGS

SEQ ID NO: 32 - protein, 20 aa, Artificial Sequence
  artificial linker sequence
GGGGSGGGGSGGGGSGGGGS

SEQ ID NO: 33 - protein, 113 aa, Artificial Sequence; identical to SEQ ID NO: 25
  artificial antibody variable chain

SEQ ID NO: 34 - protein, 121 aa, Artificial Sequence; identical to SEQ ID NO: 28
  artificial antibody variable chain

SEQ ID NO: 35 - protein, 114 aa, Artificial Sequence; identical to SEQ ID NO: 21
  artificial antibody variable chain

SEQ ID NO: 36 - protein, 123 aa, Artificial Sequence; identical to SEQ ID NO: 22
  artificial antibody variable chain

SEQ ID NO: 37 - protein, 501 aa, Artificial Sequence
  artificial scDb antibody construct
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLGGGGGSEVQLVESGGGLVQPGGSLRLSCAASGFPLSSYAMIWVRQAPGKGLEWIGMILRAGNIYYASWVKGRFTISRDNSKNTVYLQMNSLRAEDTAVYYCARRHYNREGYPIGIGDLWGQGTLVTVSSGGGGSGGGGSGGGGSGGGGSDIQMTQSPSSLSASVGDRVTITCQSSESVYNNKRLSWYQQKPGKAPKLLIYTASSLASGVPSRFSGSGSGTDFTLTISSLQPEDFATYYCQGEFTCSNADCFTFGQGTKLTVLGGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 38 - protein, 108 aa, Artificial Sequence
  artificial antibody variable chain
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKR

SEQ ID NO: 39 - protein, 116 aa, Artificial Sequence
  artificial antibody variable chain
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 40 - protein, 488 aa, Artificial Sequence
  artificial scDb antibody construct
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLGGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 41 - protein, 10 aa, Artificial Sequence
  artificial linker sequence
GGGGSGGGGS

SEQ ID NO: 42 - protein, 481 aa, Artificial Sequence
  artificial antibody light chain
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSHMDIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 43 - protein, 485 aa, Artificial Sequence
  artificial antibody VH-CH1 chain
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSHMDIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 44 - protein, 215 aa, Artificial Sequence
  artificial antibody light chain
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 219 aa, Artificial Sequence
  artificial antibody VH-CH1 chain
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 47 - protein, 95 aa, Macaca fascicularis
DGNEEMGSITQTPYQVSISGTTVILTCSQHLGSEAQWQHNGKNKEDSGDRLFLPEFSEMEQSGYYVCYPRGSNPEDASHHLYLKARVCENCMEMD

SEQ ID NO: 48 - protein, 15 aa, Artificial Sequence
  artificial peptide for epitope mapping
DGNEEMGGITQTPYK

SEQ ID NO: 49 - protein, 15 aa, Artificial Sequence
  artificial peptide for epitope mapping
GNEEMGGITQTPYKV

SEQ ID NO: 50 - protein, 15 aa, Artificial Sequence
  artificial peptide for epitope mapping
NEEMGGITQTPYKVS

SEQ ID NO: 51 - protein, 15 aa, Artificial Sequence
  artificial peptide for epitope mapping
EEMGGITQTPYKVSI

SEQ ID NO: 52 - protein, 15 aa, Artificial Sequence
  artificial peptide for epitope mapping
EMGGITQTPYKVSIS

SEQ ID NO: 53 - protein, 15 aa, Artificial Sequence
  artificial peptide for epitope mapping
MGGITQTPYKVSISG

SEQ ID NO: 54 - protein, 15 aa, Artificial Sequence
  artificial peptide for epitope mapping
GGITQTPYKVSISGT

SEQ ID NO: 55 - protein, 15 aa, Artificial Sequence
  artificial peptide for epitope mapping
GITQTPYKVSISGTT

SEQ ID NO: 56 - protein, 15 aa, Artificial Sequence
  artificial peptide for epitope mapping
ITQTPYKVSISGTTV

SEQ ID NO: 57 - protein, 15 aa, Artificial Sequence
  artificial peptide for epitope mapping
TQTPYKVSISGTTVI

SEQ ID NO: 58 - protein, 15 aa, Artificial Sequence
  artificial peptide for epitope mapping
DGNEEMGGITAAPYK

SEQ ID NO: 59 - protein, 15 aa, Artificial Sequence
  artificial peptide for epitope mapping
GNEEMGGITQAAYKV

SEQ ID NO: 60 - protein, 15 aa, Artificial Sequence
  artificial peptide for epitope mapping
NEEMGGITQTAAKVS

SEQ ID NO: 61 - protein, 15 aa, Artificial Sequence
  artificial peptide for epitope mapping
EEMGGITQTPAAVSI

SEQ ID NO: 62 - protein, 15 aa, Artificial Sequence
  artificial peptide for epitope mapping
EMGGITQTPYAASIS

SEQ ID NO: 63 - protein, 15 aa, Artificial Sequence
  artificial peptide for epitope mapping
MGGITQTPYKAAISG

SEQ ID NO: 64 - protein, 15 aa, Artificial Sequence
  artificial peptide for epitope mapping
GGITQTPYKVAASGT

SEQ ID NO: 65 - protein, 15 aa, Artificial Sequence
  artificial peptide for epitope mapping
GITQTPYKVSAAGTT

SEQ ID NO: 66 - protein, 15 aa, Artificial Sequence
  artificial peptide for epitope mapping
ITQTPYKVSIAATTV

SEQ ID NO: 67 - protein, 15 aa, Artificial Sequence
  artificial peptide for epitope mapping
TQTPYKVSISAATVI

SEQ ID NO: 68 - protein, 16 aa, Artificial Sequence
  artificial peptide for epitope mapping
EDGNEEMGGITQTPYK

SEQ ID NO: 69 - protein, 16 aa, Artificial Sequence
  artificial peptide for epitope mapping
EEEMGGITQTPYKVSI

SEQ ID NO: 70 - protein, 16 aa, Artificial Sequence
  artificial peptide for epitope mapping
EGGITQTPYKVSISGT

SEQ ID NO: 71 - protein, 16 aa, Artificial Sequence
  artificial peptide for epitope mapping
ETQTPYKVSISGTTVI

SEQ ID NO: 72 - protein, 16 aa, Artificial Sequence
  artificial peptide for epitope mapping
EPYKVSISGTTVILTC

SEQ ID NO: 73 - protein, 16 aa, Artificial Sequence
  artificial peptide for epitope mapping
EVSISGTTVILTCPQY

SEQ ID NO: 74 - protein, 16 aa, Artificial Sequence
  artificial peptide for epitope mapping
ESGTTVILTCPQYPGS

SEQ ID NO: 75 - protein, 16 aa, Artificial Sequence
  artificial peptide for epitope mapping
ETVILTCPQYPGSEIL

SEQ ID NO: 76 - protein, 16 aa, Artificial Sequence
  artificial peptide for epitope mapping
ELTCPQYPGSEILWQH

SEQ ID NO: 77 - protein, 16 aa, Artificial Sequence
  artificial peptide for epitope mapping
EPQYPGSEILWQHNDK

SEQ ID NO: 78 - protein, 16 aa, Artificial Sequence
  artificial peptide for epitope mapping
KDGNEEMGGITQTPYK

SEQ ID NO: 79 - protein, 16 aa, Artificial Sequence
  artificial peptide for epitope mapping
KEEMGGITQTPYKVSI

SEQ ID NO: 80 - protein, 16 aa, Artificial Sequence
  artificial peptide for epitope mapping
KGGITQTPYKVSISGT

SEQ ID NO: 81 - protein, 16 aa, Artificial Sequence
  artificial peptide for epitope mapping
KTQTPYKVSISGTTVI

SEQ ID NO: 82 - protein, 16 aa, Artificial Sequence
  artificial peptide for epitope mapping
KPYKVSISGTTVILTC

SEQ ID NO: 83 - protein, 16 aa, Artificial Sequence
  artificial peptide for epitope mapping
KVSISGTTVILTCPQY

SEQ ID NO: 84 - protein, 16 aa, Artificial Sequence
  artificial peptide for epitope mapping
KSGTTVILTCPQYPGS

SEQ ID NO: 85 - protein, 16 aa, Artificial Sequence
  artificial peptide for epitope mapping
KTVILTCPQYPGSEIL

SEQ ID NO: 86 - protein, 16 aa, Artificial Sequence
  artificial peptide for epitope mapping
KLTCPQYPGSEILWQH

SEQ ID NO: 87 - protein, 16 aa, Artificial Sequence
  artificial peptide for epitope mapping
KPQYPGSEILWQHNDK

SEQ ID NO: 88 - protein, 17 aa, Artificial Sequence
  artificial peptide for epitope mapping
CDGNEEMGGITQTPYKC

SEQ ID NO: 89 - protein, 17 aa, Artificial Sequence
  artificial peptide for epitope mapping
CGNEEMGGITQTPYKVC

SEQ ID NO: 90 - protein, 17 aa, Artificial Sequence
  artificial peptide for epitope mapping
CNEEMGGITQTPYKVSC

SEQ ID NO: 91 - protein, 17 aa, Artificial Sequence
  artificial peptide for epitope mapping
CEEMGGITQTPYKVSIC

SEQ ID NO: 92 - protein, 17 aa, Artificial Sequence
  artificial peptide for epitope mapping
CEMGGITQTPYKVSISC

SEQ ID NO: 93 - protein, 17 aa, Artificial Sequence
  artificial peptide for epitope mapping
CMGGITQTPYKVSISGC

SEQ ID NO: 94 - protein, 17 aa, Artificial Sequence
  artificial peptide for epitope mapping
CGGITQTPYKVSISGTC

SEQ ID NO: 95 - protein, 17 aa, Artificial Sequence
  artificial peptide for epitope mapping
CGITQTPYKVSISGTTC

SEQ ID NO: 96 - protein, 17 aa, Artificial Sequence
  artificial peptide for epitope mapping
CITQTPYKVSISGTTVC

SEQ ID NO: 97 - protein, 17 aa, Artificial Sequence
  artificial peptide for epitope mapping
CTQTPYKVSISGTTVIC

SEQ ID NO: 98 - protein, 22 aa, Artificial Sequence
  artificial peptide for epitope mapping
CDGNEEMGGITQTPYKVSISGC

SEQ ID NO: 99 - protein, 22 aa, Artificial Sequence
  artificial peptide for epitope mapping
CNEEMGGITQTPYKVSISGTTC

SEQ ID NO: 100 - protein, 22 aa, Artificial Sequence
  artificial peptide for epitope mapping
CEMGGITQTPYKVSISGTTVIC

SEQ ID NO: 101 - protein, 22 aa, Artificial Sequence
  artificial peptide for epitope mapping
CGGITQTPYKVSISGTTVILTC

SEQ ID NO: 102 - protein, 22 aa, Artificial Sequence
  artificial peptide for epitope mapping
CITQTPYKVSISGTTVILTSPC

SEQ ID NO: 103 - protein, 22 aa, Artificial Sequence
  artificial peptide for epitope mapping
CQTPYKVSISGTTVILTSPQYC

SEQ ID NO: 104 - protein, 22 aa, Artificial Sequence
  artificial peptide for epitope mapping
CPYKVSISGTTVILTSPQYPGC

SEQ ID NO: 105 - protein, 22 aa, Artificial Sequence
  artificial peptide for epitope mapping
CKVSISGTTVILTSPQYPGSEC

SEQ ID NO: 106 - protein, 22 aa, Artificial Sequence
  artificial peptide for epitope mapping
CSISGTTVILTSPQYPGSEILC

SEQ ID NO: 107 - protein, 22 aa, Artificial Sequence
  artificial peptide for epitope mapping
CSGTTVILTSPQYPGSEILWQC

SEQ ID NO: 108 - protein, 29 aa, Artificial Sequence
  artificial peptide for epitope mapping
CDGNEEMGGITQTPCDGNEEMGGITQTPC

SEQ ID NO: 109 - protein, 29 aa, Artificial Sequence
  artificial peptide for epitope mapping
CDGNEEMGSITQTPCDGNEEMGSITQTPC

SEQ ID NO: 110 - protein, 29 aa, Artificial Sequence
  artificial peptide for epitope mapping
CEEMGGITQTPYKVCDGNEEMGGITQTPC

SEQ ID NO: 111 - protein, 29 aa, Artificial Sequence
  artificial peptide for epitope mapping
CEEMGSITQTPYQVCDGNEEMGSITQTPC

SEQ ID NO: 112 - protein, 29 aa, Artificial Sequence
  artificial peptide for epitope mapping
CGGITQTPYKVSISCDGNEEMGGITQTPC

SEQ ID NO: 113 - protein, 29 aa, Artificial Sequence
  artificial peptide for epitope mapping
CGSITQTPYQVSISCDGNEEMGSITQTPC

SEQ ID NO: 114 - protein, 29 aa, Artificial Sequence
  artificial peptide for epitope mapping
CTQTPYKVSISGTTCDGNEEMGGITQTPC

SEQ ID NO: 115 - protein, 29 aa, Artificial Sequence
  artificial peptide for epitope mapping
CTQTPYQVSISGTTCDGNEEMGSITQTPC

SEQ ID NO: 116 - protein, 29 aa, Artificial Sequence
  artificial peptide for epitope mapping
CPYKVSISGTTVILCDGNEEMGGITQTPC

SEQ ID NO: 117 - protein, 29 aa, Artificial Sequence
  artificial peptide for epitope mapping
CPYQVSISGTTVILCDGNEEMGSITQTPC

SEQ ID NO: 118 - protein, 15 aa, Artificial Sequence
  artificial peptide for epitope mapping
AGNEEMGGITQTPYK

SEQ ID NO: 119 - protein, 15 aa, Artificial Sequence
  artificial peptide for epitope mapping
DGNEEMGGITQTPYK

SEQ ID NO: 120 - protein, 15 aa, Artificial Sequence
  artificial peptide for epitope mapping
GGNEEMGGITQTPYK

SEQ ID NO: 121 - protein, 15 aa, Artificial Sequence
  artificial peptide for epitope mapping
HGNEEMGGITQTPYK

SEQ ID NO: 122 - protein, 15 aa, Artificial Sequence
  artificial peptide for epitope mapping
LGNEEMGGITQTPYK

SEQ ID NO: 123 - protein, 15 aa, Artificial Sequence
  artificial peptide for epitope mapping
MGNEEMGGITQTPYK

SEQ ID NO: 124 - protein, 15 aa, Artificial Sequence
  artificial peptide for epitope mapping
NGNEEMGGITQTPYK

SEQ ID NO: 125 - protein, 15 aa, Artificial Sequence
  artificial peptide for epitope mapping
PGNEEMGGITQTPYK

SEQ ID NO: 126 - protein, 15 aa, Artificial Sequence
  artificial peptide for epitope mapping
QGNEEMGGITQTPYK

SEQ ID NO: 127 - protein, 15 aa, Artificial Sequence
  artificial peptide for epitope mapping
RGNEEMGGITQTPYK

SEQ ID NO: 128 - protein, 15 aa, Artificial Sequence
  artificial peptide for epitope mapping
AGNEEMGSITQTPYQ

SEQ ID NO: 129 - protein, 15 aa, Artificial Sequence
  artificial peptide for epitope mapping
DGNEEMGSITQTPYQ

SEQ ID NO: 130 - protein, 15 aa, Artificial Sequence
  artificial peptide for epitope mapping
GGNEEMGSITQTPYQ

SEQ ID NO: 131 - protein, 15 aa, Artificial Sequence
  artificial peptide for epitope mapping
HGNEEMGSITQTPYQ

SEQ ID NO: 132 - protein, 15 aa, Artificial Sequence
  artificial peptide for epitope mapping
LGNEEMGSITQTPYQ

SEQ ID NO: 133 - protein, 15 aa, Artificial Sequence
  artificial peptide for epitope mapping
MGNEEMGSITQTPYQ

SEQ ID NO: 134 - protein, 15 aa, Artificial Sequence
  artificial peptide for epitope mapping
NGNEEMGSITQTPYQ

SEQ ID NO: 135 - protein, 15 aa, Artificial Sequence
  artificial peptide for epitope mapping
PGNEEMGSITQTPYQ

SEQ ID NO: 136 - protein, 15 aa, Artificial Sequence
  artificial peptide for epitope mapping
QGNEEMGSITQTPYQ

SEQ ID NO: 137 - protein, 15 aa, Artificial Sequence
  artificial peptide for epitope mapping
RGNEEMGSITQTPYQ

SEQ ID NO: 138 - protein, 15 aa, Artificial Sequence
  artificial peptide for epitope mapping
AEMGGITQTPYKVSI

SEQ ID NO: 139 - protein, 15 aa, Artificial Sequence
  artificial peptide for epitope mapping
DEMGGITQTPYKVSI

SEQ ID NO: 140 - protein, 15 aa, Artificial Sequence
  artificial peptide for epitope mapping
GEMGGITQTPYKVSI

SEQ ID NO: 141 - protein, 15 aa, Artificial Sequence
  artificial peptide for epitope mapping
HEMGGITQTPYKVSI

SEQ ID NO: 142 - protein, 15 aa, Artificial Sequence
  artificial peptide for epitope mapping
LEMGGITQTPYKVSI

SEQ ID NO: 143 - protein, 15 aa, Artificial Sequence
  artificial peptide for epitope mapping
MEMGGITQTPYKVSI

SEQ ID NO: 144 - protein, 15 aa, Artificial Sequence
  artificial peptide for epitope mapping
NEMGGITQTPYKVSI

SEQ ID NO: 145 - protein, 15 aa, Artificial Sequence
  artificial peptide for epitope mapping
PEMGGITQTPYKVSI

SEQ ID NO: 146 - protein, 15 aa, Artificial Sequence
  artificial peptide for epitope mapping
QEMGGITQTPYKVSI

SEQ ID NO: 147 - protein, 15 aa, Artificial Sequence
  artificial peptide for epitope mapping
REMGGITQTPYKVSI

SEQ ID NO: 148 - protein, 15 aa, Artificial Sequence
  artificial peptide for epitope mapping
AEMGSITQTPYQVSI

SEQ ID NO: 149 - protein, 15 aa, Artificial Sequence
  artificial peptide for epitope mapping
DEMGSITQTPYQVSI

SEQ ID NO: 150 - protein, 15 aa, Artificial Sequence
  artificial peptide for epitope mapping
GEMGSITQTPYQVSI

SEQ ID NO: 151 - protein, 15 aa, Artificial Sequence
  artificial peptide for epitope mapping
HEMGSITQTPYQVSI

SEQ ID NO: 152 - protein, 15 aa, Artificial Sequence
  artificial peptide for epitope mapping
LEMGSITQTPYQVSI

SEQ ID NO: 153 - protein, 15 aa, Artificial Sequence
  artificial peptide for epitope mapping
MEMGSITQTPYQVSI

SEQ ID NO: 154 - protein, 15 aa, Artificial Sequence
  artificial peptide for epitope mapping
NEMGSITQTPYQVSI

SEQ ID NO: 155 - protein, 15 aa, Artificial Sequence
  artificial peptide for epitope mapping
PEMGSITQTPYQVSI

SEQ ID NO: 156 - protein, 15 aa, Artificial Sequence
  artificial peptide for epitope mapping
QEMGSITQTPYQVSI

SEQ ID NO: 157 - protein, 15 aa, Artificial Sequence
  artificial peptide for epitope mapping
REMGSITQTPYQVSI